(19) United States
(12) Patent Application Publication
(10) Pub. No.: US 2005/0164196 A1
(43) Pub. Date: Jul. 28, 2005

(54) Methods to predict patient responsiveness to tyrosine kinase inhibitors

(76) Inventors: Marlene Michelle Dressman, Germantown, MD (US); Lee Anne Mc Lean, Germantown, MD (US); Mihael Hristos Polymeropoulos, Potomac, MD (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/510,969

(22) PCT Filed: Apr. 16, 2003
(86) PCT No.: PCT/EP03/04007

(30) Foreign Application Priority Data
Apr. 17, 2002 (US) 60/373206
Dec. 6, 2002 (US) 60/431583

(51) Int. Cl. C12Q 1/68; G06F 19/00; G01N 33/48; G01N 33/50

(52) U.S. Cl. 435/6; 702/20

(57) ABSTRACT

This invention relates to the use of two forms of genomic analysis to predict responsiveness of patients with tyrosine kinase responsive such as Philadelphia chromosome positive leukemia to treatment with tyrosine kinase inhibitor drugs.

### BACKGROUND OF THE INVENTION

1. Field of the Invention

The invention relates to methods to predict the responsiveness of a patient with a tyrosine kinase inhibitor (TKI) responsive disease to a TKI drug. In particular, this invention relates to the use of several forms of genomic analysis to predict a patients response to TKI drugs, such as Imatinib mesylate or Imatinib or GLEEVEC® also known as GLIVEC® (also known as STI571). The type of genomic analyses includes gene expression profiling and the detection of single nucleotide polymorphisms (SNPs).

2. Description of Related Art

The human genome is now known to code for at least 96 tyrosine kinase enzymes and discontrol of any the activity of any one of these may cause disease of some form. Drugs that inhibit the activity of tyrosine kinase enzymes are proving to be extremely effective in a wide variety of disorders whose underlying pathology involves the discontrol of a tyrosine kinase somewhere in the body. These drugs include Imatinib mesylate. The disorders that are known to involve discontrol of a tyrosine kinase and are responsive to TKI drugs include, but are not limited to, chronic myelogenous leukemia (CML), Philadelphia (Ph′) chromosome-positive acute lymphoblastic leukemia, gastrointestinal stromal tumors (GIST) and various forms of hypereosinophilic syndrome. As TKI drugs are used to treat various disorders more and more disorders are found to be remarkably responsive to these drugs.

One of the first such disorders known is Ph′ chromosome-positive (Ph+) acute lymphoblastic leukemia. The various forms of leukemia comprise a variety of related disorders with similar underlying pathology. The basic pathology is a dysregulation of normal hematopoiesis. This process requires tightly regulated proliferation and differentiation of pluripotent hematopoietic stem cells that become mature peripheral blood cells. In all types of leukemia, the malignant event or events occur somewhere in the hematopoietic progression and results, by different mechanisms, in giving rise to progeny that fail to differentiate normally and instead continue to proliferate in an uncontrolled fashion. Leukemias are divided into acute and chronic types and into myeloid and lymphocytic type depending on the cell line affected and the rate of progression.

CML is also called chronic myeloid leukemia, chronic myelocytic leukemia or chronic granulocyte leukemia. CML is a disease characterized by overproduction of cells of the granulocytic, especially the neutrophilic series and occasionally the monocytic series, leading to marked splenomegaly and very high white blood cell (WBC) counts. Basophilia and thrombocytosis are common. A characteristic cytogenetic abnormality, the Ph′ chromosome, is present in the bone marrow cells in more than 95% of cases. The presence of this altered chromosome is both the key to understanding the molecular pathogenesis of this type of leukemia and a major index to assess clinical improvement in patients. See Sawyers, N. Engl. J. Med., Vol. 340, No. 17, pp. 1330-1340 (1999).

Molecular Pathogenesis

The most striking pathological feature in CML is the presence of the Ph′ chromosome in the bone marrow cells of more than 90% of patients with typical CML. The Ph′ chromosome results from a balanced translocation of material between the long arms of chromosomes 9 and 22. As more chromosomal material is lost from chromosome 22 than is gained from chromosome 9, the Ph′ chromosome is a shortened chromosome 22 containing approximately 60% of its normal complement of DNA. The break, which occurs at band q34 of the long arm of chromosome 9, allows translocation of the cellular oncogene C-ABL to a position on chromosome 22 called the breakpoint cluster region (BCR). The breakpoint in the BCR varies from patient to patient but is identical in all cells of any one patient. C-ABL is a homologue of V-ABL, the Abelson virus that causes leukemia in mice. The apposition of these two genetic sequences produces a new hybrid gene (BCR/ABL), which codes for a novel protein of molecular weight 210,000 kd (P210). The P210 protein, a tyrosine kinase, may play a role in triggering the uncontrolled proliferation of CML cells. The Ph′ chromosome occurs in erythroid, myeloid, monocytic and megakaryocytic cells, less commonly in B lymphocytes, rarely in T lymphocytes, but not In marrow fibroblasts. This extensive cellular distribution places the abnormality in CML close to the pluripotent stem cell. Studies of glucose-6-phosphate dehydrogenase (G6PD) isoenzymes support the finding of multilineage monoclonal proliferation, because a single isoenzyme is present in the aforementioned cells in some patients with CML. Insertion of a retrovirus encoding P210 (BCR/ABL) into cells of mice has led to the development of a disease closely resembling CML in some of these animals, giving credence to the hypothesis that the BCR/ABL hybrid gene is sufficient to cause CML. C-sis, the homologue of the simian sarcoma virus, is also translocated from chromosome 22 to chromosome 9 in CML but is distant from the breakpoint and not expressed in benign-phase CML. C-sis codes for a protein identical to platelet-derived growth factor (PDGF).

Although 100% of the metaphases on cytogenetic analysis usually show the presence of the Ph′ chromosome, some normal stem cells must remain. Normal diploid cells emerge on long-term bone marrow culture and after treatment with interferon, high-dose chemotherapy and autologous bone marrow transplantation.

In the past, the prognosis for CML was poor with the mean survival in Ph-positive (Ph+) CML being 3-4 years. Treatment with interferon and aggressive chemotherapy or allogeneic bone marrow transplant has improved this somewhat but the greatest improvement in the treatment of CML patients has been the introduction of Imatinib mesylate. See Druker et al., N. Engl. J. Med., Vol. 344, No. 14, pp. 1031-1037 (2001); Druker et al., N. Engi. J. Med., Vol. 344, No. 14, pp. 1038-1056 (2001); and also see Cecil Textbook of Medicine, 21st Edition, Goldman and Bennett Eds., W.B. Saunders, Chapter 176 (2000). Imatinib mesylate or Imatinib is also known as GLEEVEC®, GLIVEC® or as STI571. These terms are used hereafter interchangeably.

Imatinib mesylate is an inhibitor of the tyrosine kinase activity of several proteins that play a causative or very significant role in the development of cancers of several types. See Druker et al., Nat. Med., Vol. 2, pp. 561-566 (1996).

In CML, chromosomes 9 and 22 are truncated in the formulation of the +(9;22) reciprocal translocation that characterizes CML cells and two fusion genes are generated: BCR-ABL on the derivative 22q-chromosome, the Ph′ chromosome and ABL-BCR on chromosome 9q+. The BCR-ABL gene encodes a 210-kd protein with deregulated tyrosine kinase activity. This protein plays a pathogenetic role in CML. See Daley et al., Science, Vol. 247, pp. 824-830 (1990). Imatinib mesylate specifically inhibits the activity of this protein and other tyrosine kinases.

Imatinib mesylate has shown remarkable efficacy in treating patents with CML and in treating patients in blast crisis (BC) of CML or ALL with the Ph′ chromosome. See Druker et al. (2001), supra.

In addition, the ability of Imatinib mesylate to inhibit another tyrosine kinase that is a growth factor receptor terminal, i.e., c-Kit, allows Imatinib mesylate to be an effective treatment for a completely unrelated form of cancer, GIST. See Brief Report, Joensuu et al., N. Engl. J. Med., Vol. 344, No. 14, pp. 1052-1056 (2001).

Imatinib mesylate has been shown to be highly-effective in patients having a variety of disorders characterized by the uncontrolled activity of a tyrosine kinase. This includes Ph+leukemia. In one study of the effects of Imatinib on CML, of 54 patients who were treated with 300 mg or more, 53 had complete hematologic responses (CHR), and cytogenic responses occurred in 29 including 17 (31% of the 54 patients who received the dose) with major responses, i.e., 0-35% of cells in metaphase positive for the Ph′ chromosome; 7 of these patients had complete cytogenetic remission (CCR). See Druker et al. (2001), supra.

As used herein, a “hematologic response” is defined as a 50% reduction in the WBC count from baseline, maintained for at least two weeks.

As used herein, the term “CHR” is defined as a reduction in the WBC count to <10,000/cm and in the platelet count to <450,000/cm, maintained for at least four weeks.

Cytogenetic responses were determined by the percentage of cells in metaphase that were positive for the Ph′ chromosome in the bone marrow. As used herein “cytogenetic responses”, based on analysis of 20 cells in metaphase, is categorized as “CCR” (no cells positive for the Ph′ chromosome), “minor” (36-65% of cells positive for the Ph′ chromosome) and “absent” (over 65% of cells positive for the Ph′ chromosome). As used herein, the term “major response” is defined as a complete or a partial response.

Despite the almost 100% hematologic response, not all patients showed a CCR. Therefore, there is a need for a means to predict the efficacy of a TKI, such as Imatinib mesylate to treat a disorder in which a tyrosine kinase including, but not limited to, BCR-ABL and c-Kit plays a major causative role.

Single Nucleotide Polymorphisms (SNP)

Sequence variation in the human genome consists primarily of SNPs with the remainder of the sequence variations being short tandem repeats, including micro-satellites, long tandem repeats (mini-satellite) and other insertions and deletions. A SNP is a position at which two alternative bases occur at appreciable frequency, i.e., >1%, in the human population. A SNP is said to be “allelic” in that due to the existence of the polymorphism, some members of a species may have the unmutated sequence, i.e., the original “allele”, whereas other members may have a mutated sequence, i.e., the variant or mutant allele. In the simplest case, only one mutated sequence may exist, and the polymorphism is said to be di-allelic. The occurrence of alternative mutations can give rise to tri-allelic polymorphisms, etc. SNPs are widespread throughout the genome and SNPs that alter the function of a gene may be direct contributors to phenotypic variation. Due to their prevalence and widespread nature, SNPs have potential to be important tools for locating genes that are involved in human disease conditions (see, e.g., Wang et al., Science, Vol. 280, No. 5366, pp. 1077-1082 (1998)), which discloses a pilot study in which 2,227 SNPs were mapped over a 2.3 megabase region of DNA.

An association between a SNPs and a particular phenotype does not indicate or require that the SNP is causative of the phenotype. Instead, such an association may indicate only that the SNP is located near the site on the genome where the determining factors for the phenotype exist and therefore is more likely to be found in association with these determining factors and thus with the phenotype of interest. Thus, a SNP may be in linkage disequilibrium (LD) with the ‘true’ functional variant. LD, also known as allelic association exists when alleles at two distinct locations of the genome are more highly associated than expected. Thus a SNP may serve as a marker that has value by virtue of its proximity to a mutation that causes a particular phenotype. SNPs that are associated with disease may also have a direct effect on the function of the gene in which they are located. A sequence variant may result in an amino acid change or may alter exon-intron splicing, thereby directly modifying the relevant protein, or it may exist in a regulatory region, altering the cycle of expression or the stability of the mRNA See Nowotny, Kwon and Goate, Curr. Opin. Neurobiol., Vol. 11, No. 5, pp. 637-641 (2001).

The role that a common genomic variant might play in susceptibility to disease is best exemplified by the role that the apolipoprotein E (APOE) ε4 allele plays in Alzheimers disease (AD). The s4 allele is highly associated with the presence of AD and with earlier age of onset of disease. It is a robust association seen in many populations studied. See St George-Hyslop et al., Biol. Psychiatry, Vol. 47, No. 3, pp. 183-199 (2000). Polymorphic variation has also been implicated in stroke and cardiovascular disease (see Wu and Tsongalis, Am. J. Cardiol., Vol. 87, No. 12, pp. 1361-1366 (2001)), and in multiple sclerosis. See Oksenberg et al., J. Neuroimmunol., Vol. 113, No. 2, pp. 171-184 (2001).

It is increasingly clear that the risk of developing many common disorders and the individuals response to medication and the metabolism of medications used to treat these conditions are substantially influenced by underlying genomic variations, although the effects of any one variant might be small.

Therefore, an association between a SNP and a clinical phenotype suggests: 1) the SNP is functionally responsible for the phenotype; or 2) there are other mutations near the location of the SNP on the genome that cause the phenotype. The second possibility is based on the biology of inheritance. Large pieces of DNA are inherited and markers in close proximity to each other may not have been recombined in individuals that are unrelated for many generations, i.e., the markers are in LD.

The use of polymorphisms as genetic linkage markers is thus of critical importance in locating, identifying and characterizing the genes which are responsible for specific traits. In particular, such mapping techniques allow for the identification of genes responsible for a variety of disease or disorder-related traits including the response of the disorder to various treatments.

### SUMMARY OF THE INVENTION

The present invention, as described herein below overcomes deficiencies in the use of TKI drugs by providing a method to determine which individual with a TKI responsive disorder including, but not limited to, Ph+ leukemia, GIST, CML or hypereosinophilia will be likely to respond to a TKI drug including, but not limited to, Imatinib mesylate.

In the case of Ph+ leukemia these methods will predict which patients will respond to treatment with a TKI drug, such as Imatinib with a CCR (or CCyR) and which patients will respond with less than a CCR.

One aspect of the invention provides a method to predict which patients will respond to a tyrosine kinase inhibitor drug in Philadelphia chromosome positive leukemia patients comprising: a) determining RNA expression levels in blood for a plurality of the 55 reporter genes shown in Tables 12A and 12B; b) comparing patients gene expression profile to the mean complete cytogenetic response expression profiles shown in Tables 12A and 12B; c) determining the Pearson correlation coefficient resulting from the comparison in (b); d) determining that the patient will have complete cytogenetic response to the tyrosine kinase inhibitor if the correlation coefficient is equal to or greater than 0.57; and e) determining that the patient will be a non-responder if the correlation coefficient is less than 0.57.

Another aspect of the invention provides a method to predict which patients will respond to a tyrosine kinase inhibitor drug in Philadelphia chromosome positive leukemia patients comprising: a) determining RNA expression levels in blood for a plurality of the 55 reporter genes shown in Tables 12A and 12B; b) comparing patients gene expression profile to the mean complete cytogenetic response expression profiles shown in Tables 12A and 12B; c) determining the Pearson correlation coefficient resulting from the comparison in (b); d) determining that the patient will have complete cytogenetic response to the tyrosine kinase inhibitor if the correlation coefficient is equal to or greater than 0.54; and e) determining that the patient will be a non-responder if the correlation coefficient is less than 0.54.

In one embodiment of the invention the plurality of the 55 reporter genes comprises two or more of the 55 reporter genes shown in Tables 12A and 12B. Expression of 5 to 10, preferably of 5 to 15, 5 to 20, 5 to 25, 5 to 30, or 5 to 35, most preferred of 5 to 40 or 5 to 50, or 5 to 55 genes of Tables 12A and 12B is determined.

In another embodiment of the invention expression of at least 5, 10, 20, 30, or 40 genes as shown in Table 12A and 12B is determined. Preferably expression of at least 45 or 50 genes is analyzed. Most preferably only the 31 reporter genes in Table 12A are used. In another preferred embodiment expression of only the 55 reporter genes of Tables 12A and 12B is determined.

In a further embodiment of the invention the tyrosine kinase inhibitor is Imatinib mesylate (Imatinib or GLEEVEC® or GLIVEC® or STI571).

Another aspect of the invention relates to a method for determining the responsiveness of an individual with Philadelphia chromosome positive leukemia to treatment with a tyrosine kinase inhibitor drug comprising a) determining for the two copies of the CSK gene, present in the individual, the identity of the nucleotide pair at the polymorphic site at position 36211 of sequence AC020705.4; and b) assigning the individual to a good responder group if both pairs are AT, or if one pair is AT and one pair is GC, and to a low responder group if both pairs are GC.

A further aspect of the invention provides a method for determining the responsiveness of an individual with Philadelphia chromosome positive leukemia to treatment with a tyrosine kinase inhibitor drug comprising a) determining for the two copies of the CYP1A1 gene, present in the individual, the identity of the nucleotide pair at the polymorphic site at position 6819 in sequence X02612; and b) assigning the individual to a good responder group if both pairs are AT, and to a poor responder group if both pairs are GC, or if one is GC and one is AT.

Another aspect of the invention provides a method for determining the responsiveness of an individual with Philadelphia chromosome positive leukemia to treatment with a tyrosine kinase inhibitor drug comprising a) determining for the two copies of the IL-1β gene, present in the individual, the identity of the nucleotide pair at position 1423 of sequence X04500; and b) assigning the individual to a good responder group if both pairs are CG, and to a poor responder group if one pair is AT and one pair is CG or if both pairs are AT.

Further embodiments relate to methods wherein the tyrosine kinase inhibitor is Imatinib mesylate (Imatinib or GLEEVEC® or GLIVEC® or STI571).

Another aspect of the invention relates to a method to determine the probability of a positive clinical response in a patient, with a tyrosine kinase inhibitor drug responsive disorder, to treatment with a tyrosine kinase inhibitor drug; comprising: (a) obtaining a biological sample from the said patient, (b) determining the levels of gene expression of two or more of the 55 reporter genes listed in Tables 12A and 12B in the sample from the patent, and (c) comparing the levels of gene expression of the two or more genes determined in (b) to the levels of expression of the same genes as listed in Tables 12A and/or 12B and (d) determining the degree of similarity between the levels of gene expression of the two or more genes determined in (c), and (e) determining from the degree of similarity between the levels of gene expression of the two or more genes the probability that the patients will respond to a tyrosine kinase inhibitor drug.

Accroding to another embodiment of this aspect of the invention the two or more of the 55 reporter genes as listed in Tables 12A and 12B comprise 5 to 10, preferably 5 to 15, 5 to 20, 5 to 25, 5 to 30, or 5 to 35, most preferred 5 to 40, 5 to 50, or 5 to 55 genes of Tables 12A and 12B.

In another embodiment of the invention expression of at least 5, 10, 20, 30, or 40 genes as shown in Table 12A and 12B is determined. Preferably expression of at least 45 or 50 genes is analyzed. Most preferably only the 31 reporter genes in Table 12A are used. In another preferred embodiment expression of only the 55 reporter genes of Tables 12A and 12B is determined.

In a further embodiment of the invention the tyrosine kinase inhibitor drug responsive disorder is Philadelphia chromosome positive leukemia (Ph+ leukemia). In a preferred embodiment the tyrosine kinase inhibitor is Imatinib mesylate (Imatinib or GLEEVEC® or GLIVEC® or STI571).

Preferably the biological sample is selected from the group consisting of; a tissue biopsy, blood, serum, plasma, lymph, ascitic fluid, cystic fluid, urine, sputum, stool, salivia, bronchial aspirate, CSF or hair. In another embodiment the biological sample is a tissue biopsy cell sample or cells cultured therefrom. Most preferably is the tissue biopsy a biopsy of bone marrow or solid tissue. In another embodiment the tissue biopsy comprises cells removed from a solid tumor. In a further embodiment of the invention the biological sample are blood cells. Preferably a sample is a lysate of said cell sample.

In a further embodiment of the invention the level of gene expression is determined by measuring the level of transcription of the two or more genes in Tables 12A and/or 12B. Preferably the level of transcription is determined by measuring the level of mRNA of the two or more genes in Tables 12A and/or 12B. Alternatively the level of transcription is determined by measuring the level of cDNA corresponding to the two or more genes in Tables 12A and/or 12B. In another embodiment the step of measuring the level of transcription further comprises amplifying the mRNA or cDNA. Preferably the level of transcription is determined by techniques selected from the group of Northern blot analysis, reverse transcriptase PCR, real-time PCR, RNAse protection, and microarray.

In a preferred embodiment the level of transcription is determined for a plurality of the 55 reporter genes shown in Tables 12A and/or 12B and in a most preferred embodiment the plurality of the 55 reporter genes comprises the 31 genes shown in Table 12A. In another embodiment the plurality of the 55 reporter genes consists of the 31 genes shown in Table 12A.

In a preferred embodiment of this invention the degree of similarity in step (d) is determined by calculating a correlation coefficient whose value is a known function of the similarity of the values of gene expression of the two or more genes shown in Tables 12A and 12B, and in a most preferred embodiment the correlation coefficient is the Pearson correlation coefficient.

In another preferred embodiment of this invention if the Pearson correlation coefficient between the Mean NoCyR values of the 31 reporter genes of Table 12A and the measured values of gene expression of the same genes from a patient with a tyrosine kinase inhibitor drug responsive disorder is greater than or equal to 0.54 the patient is classified as a non-responder to treatment with a tyrosine kinase inhibitor drug and if the Pearson correlation coefficient between the Mean NoCyR values of the 31 reporter genes of Table 12A and the measured values of gene expression of the same genes from a patient with a tyrosine kinase inhibitor drug responsive disorder is less than 0.54 the patient is classified as a responder to treatment with a tyrosine kinase inhibitor drug.

In another embodiment of the invention if the Pearson correlation coefficient between the Mean NoCyR values of the 31 reporter genes of Table 12A and the measured values of gene expression of the same genes from a patent with a tyrosine kinase inhibitor drug responsive disorder is greater than or equal to 0.57 the patient is classified as a non-responder to treatment with a tyrosine kinase inhibitor drug and if the Pearson correlation coefficient between the Mean NoCyR values of the 31 reporter genes of Table 12A and the measured values of gene expression of the same genes from a patient with a tyrosine kinase inhibitor drug responsive disorder is less than 0.57 the patient is classified as a responder to treatment with a tyrosine kinase inhibitor drug.

Thus in one most preferred embodiment, this invention provides a method to predict which patients will respond to a TKI drug in Ph+ leukemia patients comprising: determining RNA expression levels in blood for a plurality of the 55 (or for the 31 most preferred reporter genes) reporter genes shown in Tables 12A and 12B; comparing patients gene expression profile to the mean CCR expression profiles shown in Tables 12A and 12B; determining the Pearson Correlation Coefficient (PCC) resulting from the comparison; determining that the patient will have CCR to the TKI if the correlation coefficient (CC) is ≧0.54 or ≧0.57; and determining that the patient will be a non-responder if the CC is <0.54 or <0.57, respectively.

In some preferred embodiment of this invention the method of determining the levels of gene expression of two or more of the 55 reporter genes listed in Tables 12A and 12B in the sample from the patient comprises determining presence and levels of expression of the polypeptides corresponding to the two or more of the 55 reporter genes listed in Tables 12A and/or 12B. In a preferred embodiment of the invention the presence and the levels of expression of the polypeptides of the said genes are detected by using a reagent which specifically binds to said polypeptides. Most preferably the reagent is selected from the group consisting of an antibody, an antibody derivative, and an antibody fragment. In one embodiment of the invention the presence and the levels of expression of the polypeptides of the said genes are detected through Western blotting using a labeled probe specific for each polypeptide. The labeled probe is preferably an antibody, most preferred is a monoclonal antibody.

Another aspect of this invention provides methods for determining the responsiveness of a patient with a tyrosine kinase inhibitor drug responsive disorder, to treatment with a tyrosine kinase inhibitor drug comprising: (a) determining for the two copies of the putative gene DKFZP434C131 in the 15q22.33 region, present in the said patient, the identity of the nucleotide pair at the polymorphic site referred to as the rs2290573 polymorphism; and (b) assigning the individual to a good responder group if both pairs are AT, or if one pair is AT and one pair is GC, and to a low responder group if both pairs are GC.

A further aspect of the present invention provides methods for determining the responsiveness of a patient with a tyrosine kinase inhibitor drug responsive disorder, to treatment with a tyrosine kinase inhibitor drug comprising: (a) determining for the two copies of the CYP1A1 gene, present in the said patient, the Identity of the nucleotide pair at the polymorphic site at position 6819 in sequence X02612; and (b) assigning the individual to a good responder group if both pairs are AT, and to a poor responder group if both pairs are GC, or if one is GC and one is AT.

Another aspect of the present invention relates to a method for determining the responsiveness of a patient with a tyrosine kinase inhibitor drug responsive disorder, to treatment with a tyrosine kinase inhibitor drug comprising: (a) determining for the two copies of the IL-1beta gene, present in the patient, the identity of the nucleotide pair at position 1423 of sequence X04500; and (b) assigning the individual to a good responder group if both pairs are CG, and to a poor responder group if one pair is AT and one pair is CG or if both pairs are AT.

In further embodiments of the invention the tyrosine kinase inhibitor drug responsive disorder is Philadelphia chromosome positive leukemia, and the tyrosine kinase inhibitor is Imatinib mesylate (Imatinib or GLEEVEC® or GLIVEC® or STI571). Preferably the methods are performed ex-vivo.

Another aspect of the invention provides a kit for determining the responsiveness to treatment with a tyrosine kinase inhibitor drug of a patient with a tyrosine kinase inhibitor drug responsive disorder, comprising: a means for detecting the polypeptides corresponding to the two or more of the 55 reporter genes listed in Tables 12A and/or 3B. The means for detecting the polypeptides comprise preferably antibodies, antibody derivatives, or antibody fragments. The polypeptides are most preferred detected through Western blotting utilizing a labeled antibody. In another embodiment of the invention the kit further comprising means for obtaining a biological sample of the patient. Preferred is a kit which further comprises a container suitable for containing the means for detecting the polypeptides and the biological sample of the patient, and most preferably further comprises instructions for use and interpretation of the kit results.

In a preferred embodiment the kit for use in determining treatment strategy for a patient with a tyrosine kinase inhibitor drug responsive disorder, comprises: (a) a means for detecting the polypeptides corresponding to the two or more of the 55 reporter genes listed in Tables 12A and/or 12B; (b) a container suitable for containing the said means and the biological sample of the patient comprising the polypeptides wherein the means can form complexes with the polypeptides; (c) a means to detect the complexes of (b); and optionally (d) instructions for use and interpretation of the kit results.

Another aspect of the invention relates to a kit for determining the responsiveness to treatment with a tyrosine kinase inhibitor drug, of a patient with a tyrosine kinase inhibitor drug responsive disorder, comprising: a means for measuring the level of transcription of the two or more genes listed in Tables 12A and/or 12B. In a preferred embodiment the means for measuring the level of transcription comprise oligonucleotides or polynucleotides able to bind to the transcription products of said genes. Preferably the oligonucleotides or polynucleotides are able to bind mRNA or cDNA corresponding to said genes. In a most preferred embodiment the level of transcription is determined by techniques selected from the group of Northern blot analysis, reverse transcriptase PCR, real-time PCR, RNAse protection, and microarray. In another embodiment of the invention the kit further comprising means for obtaining a biological sample of the patient. Preferred is a kit which further comprises a container suitable for containing the means for measuring the level of transcription and the biological sample of the patient, and most preferably further comprises instructions for use and interpretation of the kit results.

In a preferred embodiment the kit for determining the responsiveness to treatment with a tyrosine kinase inhibitor drug, of a patient with a tyrosine kinase inhibitor drug responsive disorder; comprises (a) a number of oligonucleotides or polynucleotides able to bind to the transcription products of the two or more genes listed in Tables 12A and/or 12B; (b) a container suitable for containing the oligonucleotides or polynucleotides and the biological sample of the patient comprising the transcription products wherein the oligonucleotides or polynucleotide can bind to the transcription products, (c) means to detect the binding of (b); and optionally, (d) instructions for use and interpretation of the kit results.

Most preferably a kit according to the embodiments of the present invention is used for the determination step (b) of the methods according to other aspects of the invention.

Another preferred aspect of this invention provides a kit for the identification of a polymorphic site of the putative gene DKFZP434C131 in the 15q22.33 region of a patient with a tyrosine kinase inhibitor drug responsive disorder, wherein the kit comprises a means for determining the genetic polymorphism pattern at the two polymorphic sites of the putative gene DKFZP434C131 in the 15q22.33 region.

In another aspect of the invention a kit for the identification a polymorphism pattern at the CYP1A1 gene of a patient with a tyrosine kinase inhibitor drug responsive disorder is provided, said kit comprising a means for determining the genetic polymorphism pattern at the CYP1A1 gene polymorphic site at position 6819 in sequence X02612.

A further aspect of the invention relates to a kit for the identification of a polymorphism pattern at the IL-1 beta gene of a patient with a tyrosine kinase inhibitor drug responsive disorder, said kit comprising a means for determining the genetic polymorphism pattern at the IL-1 beta gene at position 1423 in sequence X04500.

In preferred embodiments the kits further comprise a means for obtaining a biological sample of the patient, most preferable the means comprises a DNA sample collecting means.

In various embodiments this invention provides kits wherein the means for determining a genetic polymorphism pattern at the specific polymorphic site comprise at least one gene specific genotyping oligonucleotide and kits wherein the means for determining a genetic polymorphism pattern at the specific polymorphic site comprise two gene specific genotyping oligonucleotides and kits wherein the means for determining a genetic polymorphism pattern at the polymorphic site comprise at least one gene specific genotyping primer composition comprising at least one gene specific genotyping oligonucleotide. In further embodiments this invention provides kits wherein the genotyping primer composition comprises at least two sets of allele specific primer pairs and kits wherein the two genotyping oligonucleotides are packaged in separate containers.

In preferred embodiments of this invention the determination step (a) referred to above employs the use of a kits according to the invention.

### BRIEF DESCRIPTION OF THE DRAWINGS

FIG. 1: Optimization of Number of Reporter Genes. Plot of error rates as a function of number of genes for determination of optimum number of genes. Calculated values by increasing number of individual genes from 5-55. Arrow indicates the optimum number of genes (N=31) that minimizes false negatives (CCyR misclassified as NoCCyR: N=1) and false positives (NoCCyR misclassified as CCyR: N=4).

FIG. 2: Determination of Threshold Correlation Value. PCC calculated using mean NoCCyR expression profile of optimized set of 31 genes. False negative equals a patient with CCyR who was misclassified as NoCCyR based upon their expression profile. False positive equals a patient with NoCCyR who was misclassified as having a CCyR expression profile. Arrows indicate threshold correlation values at optimal accuracy (minimum false negatives (N=2) and false positives (N=2); r=0.57) and optimized specificity (less than 10% false positives (N=1); r=0.54).

FIG. 3: Cluster of 31 Reporter Genes. Grey scale represent relative levels of expression, with dark representing low expression and light representing high expression. Samples are ordered according to correlation of gene expression with the mean NOCyR expression profile and clustering of genes was performed using the Pearson similarity method in GENE SPRING®. CCs for each sample are plotted in the middle panel, with the highest correlation at the top. The right panel represents the actual CCyR status, with solid indicating CCyR and white representing patients with NoCCyR. Solid line indicates the threshold that was determined to minimize false negatives to <10% (r=0.540) and was used for subsequent analyses. Dashed line indicates threshold value that further increased specificity by reducing false negatives to zero (r=0.437).

FIG. 4: Association Between Genotype of the rs2290573 Polymorphism and Cytogenetic Response (OKR). The odds ratio (OR) from the association of the polymorphism mapped to the putative gene with OKR is 4.69 (95% CI: 1.23, 17.76). The p-value associated with this graph is 0.00036.

FIG. 5: Genetic Map of 15q22.33 from NCBI Map View Build 30.

FIG. 6: Association of CYP1A1Locus with CHR. The OR from the association of the CYP1A1locus with CHR is 12.7 (95% CI: 2.6-62.1). The p-value associated with this graph is 0.004.

FIG. 7: Association of IL-1 beta Locus with MCyR. The OR from the association of the IL-1 beta locus with MCyR is 3.0 (95% CI: 1.2-7.4). The p-value associated with this graph is 0.0121.

FIG. 8: Time to Progression (TTP) as a Function of rs2290573 Polymorphism Genotype. Survival analysis plot indicating time to progression (TTP), in months, using 18-month data. Six of 26 imatinib-treated patients (23.1%) with a CC genotype and four of 79 patients (5.1%) with a CT or TT genotype for the rs2290573 polymorphism experienced progression events. A significant difference was observed between genotypes according to the Log-Rank (0.0041) and Wilcoxon (0.0049) statistical tests.

### DETAILED DESCRIPTION OF THE INVENTION

The present invention provides several methods to predict or estimate the likelihood or probability that a patient with a TKI responsive disorder will respond with positive or favorable clinical results to treatment with a TKI drug, medication or other TKI treatment. These methods involve several forms of genomic or genetic analysis.

In one aspect of the invention, the degree of gene expression of a number of identified genes is measured. The level of gene expression of these genes is able to distinguish between those patients who will respond well and those patients who will not respond well to a TKI drug.

In practice, the pattern of the expression of two or more of these listed genes in a patient whose response status is unknown is compared to the pattern of the same genes in patients whose response statue is known. The mathematical similarity between the two patterns determines the probability that the unknown patients response will be similar to response of the known patient. The discovery and identification of these genes form part of the basis of this invention. In various embodiments the gene expression pattern can be determined in a wide variety of ways including, but not limited to, measuring mRNA levels in tissue or body fluids or measuring protein expression products in tissue or body fluids including, but not limited to blood, lymph, urine, bile, CSF sweat, serum, stool, salvia or in biopsy material including but not limited to bone marrow aspirates and solid tumors.

One prefered aspect of the present invention provides methods to predict the response of a patient with a TKI responsive disorder including, but not limited to, Ph+leukemia to a TKI drug including, but not limited to, Imatinib mesylate (GLIVEC® or GLEEVEC®). The determination can be performed on a sample from the patient including, but not limited to, biopsy tissue or blood, serum or some other body fluid.

In another aspect, this invention provides a different method to predict the likelihood or probability that a patient with a TKI responsive disorder will respond with a positive clinical results to treatment with a TKI drug, medication or treatment. These methods involve genetic analysis in the form of the detection of one or more SNPs in the patients genome. The novel discovery that the presence of these SNPs can predict the response of a patient with a TKI responsive disorder to treatment with a TKI drug, medication or treatment, forms part of the basis of this invention.

In one preferred embodiment, the presence or absence of these identified SNPs can be used to predict the response of a patient with Ph+ leukemia to the TKI Imatinib mesylate (GLIVEC® or GLEEVEC® or STI571). In the example shown, this response is measured in the form of a CHR and a major cytogenetic response (MCyR) in patients with Ph+ leukemia when treated with Imatinib mesylate. This method involves the determination of the presence of absence of specific SNPs in one or more of three polymorphic sites in the following genes:
1) The G→A change at position 6819 in sequence X02612 of the CYP1A1gene, the presence of this polymorphism in the CYP1A1 gene produces a Ile to Val change at amino acid position 462 in the expressed protein.
2) The rs2290573 polymorphism, which is currently mapped to the putative gene, DKFZP434C131 in the 15q22.33 region, this putative gene codes for a tyrosine kinase. (This polymorphism was formally mapped to the CYP1A1gene and was referred to as the CSK gene and described as a C→T at position 36211 of sequence AC020705.4, with no amino add change in the expressed protein).
3) The polymorphism C→T at nucleotide position −511 of the IL-1 beta gene in the promoter region; with no amino acid change in the expressed protein; or a C→T at nucleotide position 1423 of sequence X04500.

As used herein, the term “tyrosine kinase inhibitor” or “TKI” means any substance or compound which is capable of causing effective inhibition of a tyrosine kinase enzyme. This includes, but is not limited to, small molecule drugs, such as Imatinib (Imatinib mesylate or GLIVEC® or GLEEVEC® or STI571, Novartis Pharmaceutical Corporation, Basel, Switzerland).

As used herein, the term ““TKI responsive disease” means any disease the course or progression of which can be controlled, improved or favorably altered in any way by the action of a TKI drug. In general, these diseases involve mutations or other discontrol mechanisms that result in constitutive activity of various tyrosine kinase enzymes and result in ligand independent tyrosine kinase activity including autophosphorylation of the enzyme with resulting uncontrolled cell proliferation and simulation of downstream signaling pathways. The tyrosine kinases that may be involved include, but are not limited to, ABL and the BCR-ABL fusion protein of CML and Ph+ acute lymphoblastic leukemia; PDGF receptor and the product of the c-kit gene. The diseases known to be TKI responsive diseases include, but are not limited to, CML, Ph+ acute lymphoblastic leukemia, GIST and various forms of hypereosinophilic syndrome.

However, the human genome is now known to code for at least 96 tyrosine kinase enzymes and similar discontrol of any one of these may cause disease of some form. The term “TKI responsive disease” is meant to encompass any disease the course or progress of which can be favorably altered by the inhibition of any tyrosine kinase enzyme known today or discovered in the future.

This invention is based, in part, on the discovery of approximately 55 genes which are differentially-expressed in tissue from patients with Ph+ leukemia who will respond to treatment with a TKI drug with a CCR and those patients who will respond with less than a CCR. The methods of this invention comprise measuring the activities of two or more of the approximately 55 genes that are shown to be differently-expressed in tissue from patients who will respond to treatment with a TKI drug with a CCR and those patients who will respond with less than a CCR and comparing the patterns of gene activity with a patient whose TKI response is unknown.

In a preferred embodiment, only a portion of the 55 genes would be measured. These measurements, could, in various embodiments, be in the tissue itself from biopsies or in blood or serum, etc, or in preferred embodiments, could be performed as more indirect measurement of gene expression, including but not limited to, cRNA or polypeptide expression products in various tissues including blood or other body fluids.

The measurements, direct or indirect, of the rates of expression of two or more of these 55 genes from an individual whose response status is unknown could then be compared to the expression values for the same two or more genes measured in patients whose response status is known.

The “degree of similarity” (DOS) of the unknown two or more gene expression values to the gene expression values of the same two or more genes in patients who did or did not have a CCR when treated with a TKI drug would then be determined.

This DOS could be determined by any procedure that produces a result whose value is a known function of the degree of similarity between the two groups of numbers, i.e., the measured gene expression values of the two or more genes in tissue from an individual whose TKI drug response status is unknown and to be determined and the measured gene expression values for the same two or more genes from individuals whose TKI drug response status is known.

As used herein, the term “DOS” shall mean the extent to which the pattern of gene expression values are alike or numerically similar, as measured by a comparison of the values of gene expression determined by direct or indirect methods.

In a preferred embodiment, the DOS would be determined by a mathematical calculation resulting in a correlation coefficient (CC). In a particularly preferred embodiment, the Pearson correlation coefficient (PCC) would be determined but any other mathematical procedure that produces a result whose value is a known function of the DOS between the two groups of numbers could be used. Many such procedures are known to those of skill in the art.

The value of the DOS (PCC) so calculated can then be directly-related to the probability that the sample is from a patient who will or will not have a positive or negative clinical response when treated with a TKI drug. That is to say, the higher the patients' “DOS” (CC or PCC) as compared to the gene expression values from a patient who is known not to have had a good response or the higher the “DOS” (CC or PCC) as compared to the gene expression values from a patient is known to have had a good response when treated with a TKI drug then the greater the probability that the patient will not or will have a similar response when they are treated with a TKI drug.

In a preferred embodiment the methods are used to predict the response of a patient with Ph+ leukemia to a TKI drug such as Imatinib mesylate and the definition of a good response is the achievement of a complete cytogenic response or CCR, while the definition of a poor response is the failure to achieve a CCR.

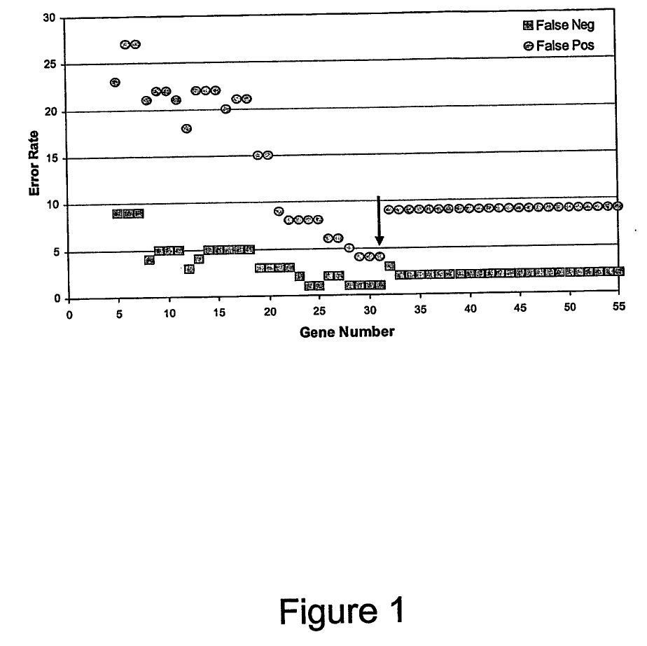

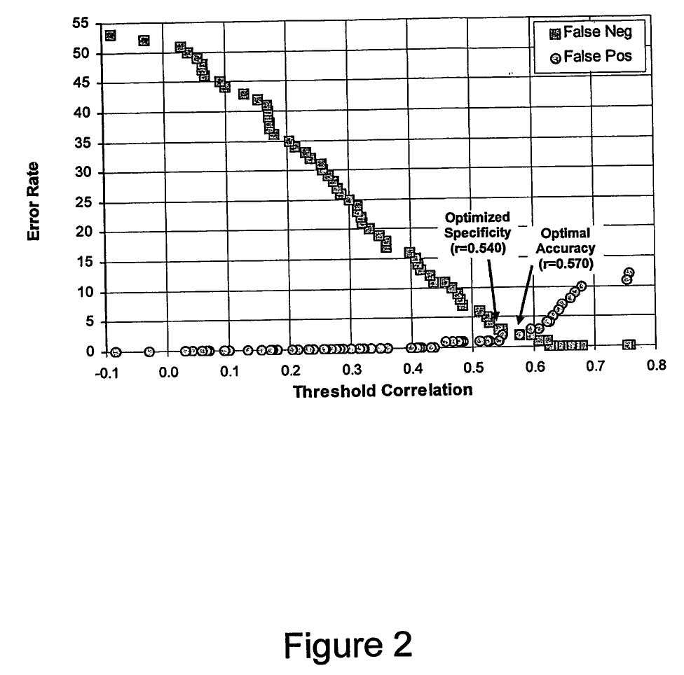

Thus, in a given case the value of the DOS, can be used to determine probabilities for the type of response to be expected. Those of skill in the art will understand that the clinical circumstance for each patient will dictate the value of the DOS (PCC) to be used as a cutoff or to help make clinical decisions with regard to a specific patient. For example, in one embodiment, it is desirable to determine with optimal accuracy the number of a group of patients who will have a good response to a TKI drug. For example, a large percentage of patients with Ph+ leukemia will respond extremely well to treatment with Imatinib mesylate or GLEEVEC®, however, some patients do not respond fully and the time lost In completing a trial of a TKI will delay the decision to begin other treatments such as a bone marrow transplant. In this situation it is important to determine ahead of time which patient is likely to show a good response, i.e., achieve a CCR and which patients will probably not respond fully. However, in this situation both false positives (patients who will have not have a CCR misclassified as patients who will have a CCR) and false negatives (a patient who will respond with a CCR misclassified as patient who would not not be expected to have a CCR response when treated with a TKI drug) are disadvantageous. The false positives may result in an unnecessary and time consuming drug trial that will prove unsuccessful and the false negatives may result in a patient who would respond well not being treated with the TKI This means that it is desirable, in this situation to minimize both false positives and at the same time to minimize false negatives, i.e., maximize accuracy. The determination of the optimal number of discrimating genes to use and the correct value of the PCC are shown in FIGS. 1 and 2.

Another example of this would be one preferred embodiment of the present invention where it is desired to divide up a patient population into responders and non-responders, this would work as shown in FIG. 1 and FIG. 2, using the optimized 31 predictor gene set shown in Table 12A (as described below) if the patients gene expression profile correlates with the mean No Complete Cytogenetic Response (NoCCyR) shown in Tables 12A with a PCC greater than 0.570, then the patient will be considered a non-responder to treatment with a TKI drug.

If the patients gene expression profile correlates with the mean NoCCyR expression with a CC of 0.57 or less than the patient will be considered a responder to treatment with a TKI drug.

In another preferred embodiment, the value of the PCC can be set to produce optional sensitivity. That is, to make the smallest possible number of false positives (a non-responder misclassified as a good responder). Such an optimal sensitivity setting would be indicated In situations where the determination of whether or not a given patient will be a good responder must be made with the greatest certainty obtainable. In this embodiment, the threshold for optimized specificity is determined by setting the PCC to a threshold of 0.5400. In the example shown below this value of PCC reduced the number of false positive (N=1) while only slightest increasing the number of false negatives (N=3). Using a threshold at r=0.437 served to further Increase the specificity and eliminated all false positives.

One of skill in the art can readily see that the value of the CC used will determine the relative numbers of false positives as compared with false negatives and this value can therefore be chosen to meet the individual clinical needs of the patient. For example, in the example shown the optimum accuracy was found at a PCC of 0.540.

As used herein the term “optimum accuracy” shall mean the condition in which the number of false positives and false negatives are both minimized.

In another preferred embodiment, the value of the CC can be set to produce optimal sensitivity. That is, to make the smallest possible number of false negatives (a good responder misclassified as a non-responder). Such an optimal sensitivity setting would be indicated in situations where the determination of whether or not a given patient will be a good responder must be made with the greatest certainty obtainable. In this embodiment, the threshold is determined by setting the CC to a threshold of 0.620. In the example given, using the 31 gene set predictor probes shown in Table 12A 100% of patients with a CC of greater than 0.620 as compared to the NoCCyR group turned out to be poor responders.

As is shown in the example, one of skill in the art can choose a PCC that will either maximize sensitivity or maximize specificity or accuracy or produce any desired ratio of false positives or false negatives. One of skill in the art can easily adjust their choice of PCC to the clinical situation to provide maximum benefit and safety to the patient.

Thus, in a preferred embodiment the present invention provides methods to predict the response of a patient with a TKI responsive disorder, such as Ph+ leukemia to a TKI drug, such as Imatinib mesylate or GLIVEC®.

In one embodiment, a patient who is a potential candidate for GLIVEC® would have blood drawn for a determination of a RNA expression profile comprising two or more of the 55 reporter genes shown in Tables 12A and 12B. The RNA expression levels of this group of genes from the candidate would be compared to the mean CCR expression levels for the same genes as shown in Tables 12A and 12B and the PCC calculated. If the coefficient is ≧0.57 or ≧0.54, then the candidate will be expected to have a CCR following treatment with GLIVEC® (STI571). If the coefficient is <0.57 or <0.54 respectively, then the candidate would be predicted to be a non-responder. In one embodiment the RNA expression profile of a plurality of the genes in Tables 12A and 12B would be determined and compared.

In a more preferred embodiment, the RNA expression profile of all 55 genes shown in Tables 12A and 12B would be determined and compared. In a another preferred embodiment, the RNA expression profile of a plurality of the 31 genes shown in Table 12A would be determined and compared. In the most preferred embodiment, the RNA expression profile of all genes 1-31 shown in Table 12A would be determined and compared to the measured mean CCR expression values in Table 12A.

Obviously, other CCs can be used and will provide different error rates as shown in FIG. 2. One of skill in the art may choose the PCC to use as a cut-off value depending on the objective of the determination. For example, a higher rate would increase the number of false positives but reduce the number of false negatives. Use of a lower PCC would conversely increase the number of false negatives but reduce the number of false positives. The optimum value of 0.57 calculated using the mean NoCCyR expression profile of the most preferred 31 genes shown in Table 12A is to optimize accuracy and the use of a value of 0.54 would (in this situation) optimize specificity (minimize false positives).

Determination of TKI Response Using SNP's

In another embodiment, this invention provides methods to predict the likelihood of a CHR and a MCyR in patients with Ph+ leukemia when treated with GLIVEC®. This method involves the determination of the presence of absence of specific SNPs in one or more of three genes, i.e., CYP1A1, IL-1beta and the rs2290573 polymorphism which is currently mapped to the putative gene DKRZP434C131.

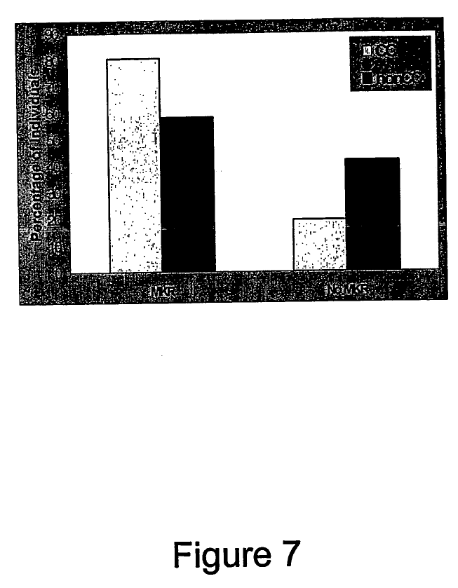

In one embodiment, the presence of a polymorphism C→T at nucleotide position −511 (promoter region; no amino acid change); or (C→T at nucleotide position 1423 of sequence X04500) in the IL-1 beta gene predicts the likelihood of the patient having a MCyR as shown in FIG. 7 and Table 3.

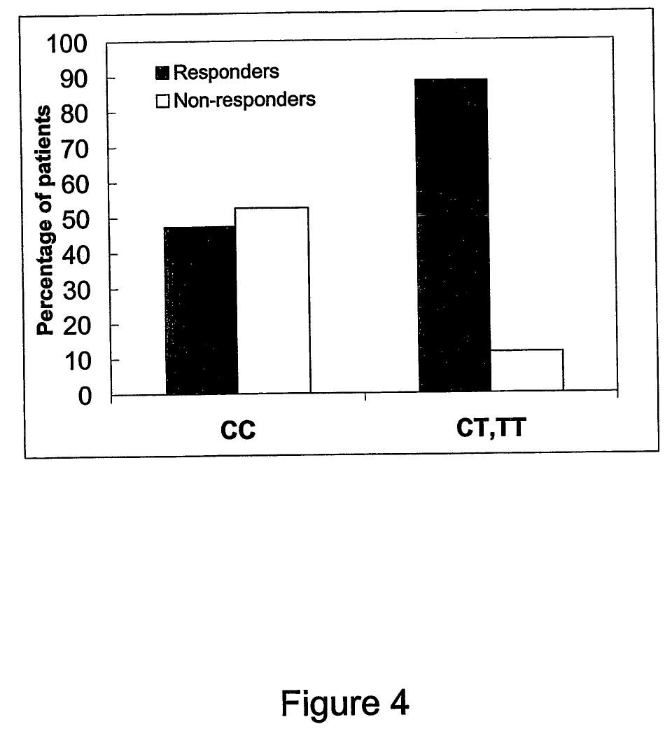

In another embodiment, the presence of a polymorphism in the rs2290573 polymorphism site which is currently mapped to the putative gene DKRZP434C131 significantly predicts the likelihood of a MCyR in a patient as shown in FIG. 4 and Table 3. This putative gene codes for a tyrosine kinase.

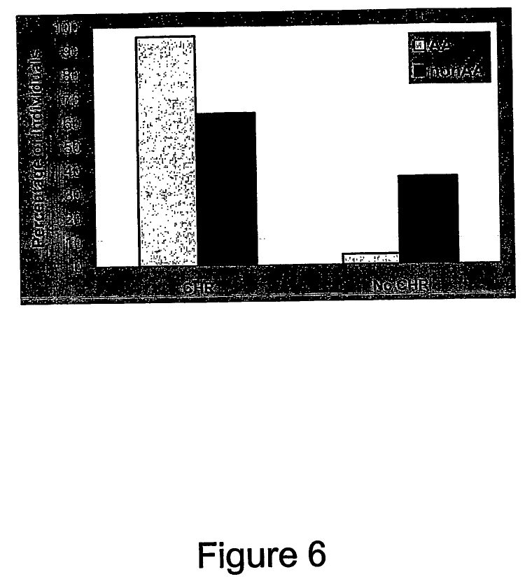

In a further embodiment, the presence of a polymorphism in the CYP1A1 gene (which produces a lie to Val change at amino acid position 462 in the expressed protein or a G→A change at position 6819 in sequence X02612 of the nucleotide) in a candidate for GLIVEC® is determined. If this polymorphism is detected, the likelihood of the patient showing a CHR is markedly reduced as shown in FIG. 6 and Table 3.

### EXAMPLE 1

Method One Use of SNP's

Identification and Characterization of SNPs

Many different techniques can be used to identify and characterize SNPs, including single-strand conformation polymorphism analysis, heteroduplex analysis by denaturing high-performance liquid chromatography (DHPLC), direct DNA sequencing and computational methods. See Shi, Clin. Chem., Vol. 47, No. 2, pp. 164-172 (2001). Thanks to the wealth of sequence information in public databases, computational tools can be used to identify SNPs in silico by aligning independently submitted sequences for a given gene (either cDNA or genomic sequences). Comparison of SNPs obtained experimentally and by in silico methods showed that 55% of candidate SNPs found by SNPFinder (http://lpgws.nci.nih.gov:82/perl/snp/snp_cgi.pl) have also been discovered experimentally. See Cox, Boillot and Canzian, Hum. Mutat., Vol. 17, No. 2, pp. 141-150 (2001). However, these in silico methods could only find 27% of true SNPs.

The most common SNP typing methods currently include hybridization, primer extension and cleavage methods. Each of these methods must be connected to an appropriate detection system. Detection technologies include fluorescent polarization (see Chen, Levine and Kwok, Genome Res., Vol. 9, No. 5, pp. 492498 (1999)), luminometric detection of pyrophosphate release (pyrosequencing) (see Ahmadian et al., Anal. Biochem., Vol. 280, No. 1, pp. 103-110 (2000)), fluorescence resonance energy transfer (FRET)-based cleavage assays, DHPLC and mass spectrometry. See Shi (2001), supra and U.S. Pat. No. 6,300,076 B1. Other methods of detecting and characterizing SNPs are those disclosed in U.S. Pat. Nos. 6,297,018 B1 and 6,300,063 B1. The disclosures of the above references are incorporated herein by reference in their entirety.

In particularly preferred embodiments of this invention, the detection of the polymorphism can be accomplished by means of so-called INVADER™ technology (available from Third Wave Technologies Inc., Madison, Wis.). In this assay, a specific upstream “Invader” oligonucleotide and a partially overlapping downstream probe together form a specific structure when bound to complementary DNA template. This structure is recognized and cut at a specific site by the Cleavase enzyme, and this results in the release of the 5′ flap of the probe oligonucleotide. This fragment then serves as the “invader” oligonucleotide with respect to synthetic secondary targets and secondary fluorescently-labeled signal probes contained in the reaction mixture. This results in specific cleavage of the secondary signal probes by the Cleavase enzyme. Fluoresence signal is generated when this secondary probe, labeled with dye molecules capable of fluorescence resonance energy transfer, is cleaved. Cleavases have stringent requirements relative to the structure formed by the overlapping DNA sequences or flaps and can, therefore, be used to specifically detect single base pair mismatches immediately upstream of the cleavage site on the downstream DNA strand. See Ryan et al., Mol. Diagn., Vol. 4, No. 2, pp. 135-144 (1999); Lyamichev et al., Nat. Biotechnol., Vol. 17, No. 3, pp. 292-296 (1999); and also U.S. Pat. Nos. 5,846,717 and 6,001,567, the disclosures of which are incorporated herein by reference in their entirety.

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

Utilization of SNPs for Predication of Response

Pharmacogenetic analysis was conducted to identify genetic factors that associate with MCyR in the clinical trial CSTI571 0106, also known as the International Randomized Study of Interferon vs. STI571 (IRIS) Study. Sixty-eight (68) polymorphic loci in 26 genes were examined and a significant association in Imatinib-treated patients between best confirmed cytogenetic response (OKR) and the rs2290573 polymorphism mapped to 15q22.33 was observed (p=0.00036, Bonferroni corrected p=0.024). Individuals with a CC genotype at this locus had a major cytogenic response (McyR) rate of 47% when treated with Imatinib, while individuals with a CT or TT genotype had a response rate of 88% when treated with Imatinib (OR: 4.69; 95% CI: 1.24, 17.76).

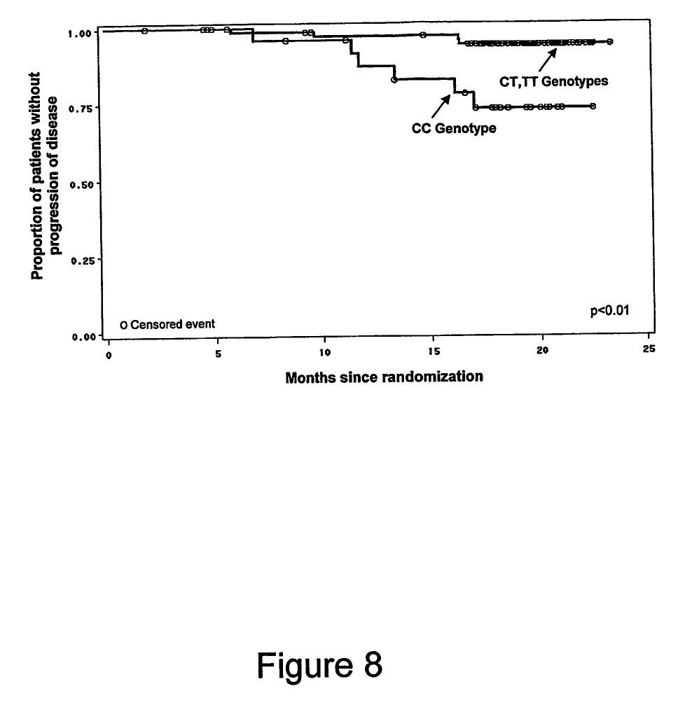

A significant association was also seen between this polymorphism and best cytogenetic response-unconfirmed (BKR) (p=0.019). The association was significant with 18-month follow-up data. Time to progression (TTP) analysis illustrated a significant difference in progression events based on genotype for the rs2290573 polymorphism, see FIG. 8.

Furthermore, an additional association was detected between an IL-1 beta polymorphism, which is in LD with a TATA-box polymorphism, and MCyR in Imatinib-treated patients (p=0.014, Bonferroni corrected p-value=0.95). CML patients with a CC genotype at the C-511 T IL1-beta locus had a MCyR rate of 94%, while patients with a CT or TT genotype had a response rate of 72.4% (OR: 5.03; 95% CI: 1.21, 20.81).

In addition, a significant association was observed between response and polymorphisms in the CYP1A1gene in imatiab mesylate-treated individuals. In addition to associations between response and the above polymorphisms, these polymorphisms are associated with the time to progression of the disease. Therefore an individual's genotype could be used as a predictive marker of TTP of disease.

Two different association studies were done based on patient information from the study, including all response data. The association studies were performed based on the responses of CHR and MCyR. Polymorphisms within the IL-1 beta gene and the rs2290573 polymorphism, (formerly referred to as a polymorphism within the CSK gene), were significantly-associated with MCyR (p=0.0121 and p=0.004). The CYP1A1 polymorphism was significantly-associated with CHR (p=0.004).

The results of this study suggest that a polymorphism within the CYP1A1 gene can be used as a predictive marker for CHR in STI571-treated patients. Additionally, the results of this study identified polymorphisms in the IL-1 beta promoter and in the putative gene, DKFZP434C131 in the 15q22.33 region, i.e., the rs2290573 polymorphism, that have potential to be used as predictive markers for MCyR.

Pharmacogenetic analysis to identify predictive markers of hematological response and/or cytogenetic response was conducted in Phase III clinical trial CSTI571 0106. CSTI571 0106 was a study of STI571 vs. IFN-alpha combined with cytarabine arabinoside (Ara-C) in patients with newly-diagnosed, previously untreated Ph+ CML-CP.

Therefore, the association between MCyR and the rs2290573 polymorphism located on 15q22.33 is striking and novel and partly forms the basis of this invention. The rs2290573 polymorphism mapped to the intronic region of putative gene DKFZP434C131, which contains a tyrosine kinase region, can be considered as a genetic marker for cytogenetic response in Imatinib-treated patients.

The Ph′ chromosome, the hallmark of CML, yields a fusion protein, BCR-ABL, that acts constitutively on a number of cell processes linked to the proliferation of leukemic cells. Imatinib mesylate (GLEEVEC®, GLIVEC® or STI571) inhibits the tyrosine kinase activity of the BCR-ABL fusion protein so that the proliferation of Ph+ cells is arrested.

Pharmacogenetic analysis to identify genetic markers of cytogenetic response was conducted in clinical trial CSTI571 0106. This was a Phase III study of Imatinib vs. interferon-alpha (IFN-alpha combined with Ara-C) in patients with newly diagnosed, previously untreated Ph+ CML-CP. The pharmacogenetics protocol was offered at U.S. centers only. A total of 109 patients treated with Imatinib or IFN-alpha 57.72% of U.S. patients consented to participate in this pharmacogenetic analysis.

The primary objective of the CSTI571 0106 trial was to determine the TTP in adult patients with newly-diagnosed, previously-untreated Ph+ CML randomized to treatment with Imatinib compared to patients randomized to treatment with IFN-alpha+Ara-C. See Gathmann, Reese and Wehrle, and O'Brien et al. N. Engl. J. Med., Vol. 348, pp. 994-1004 (2003).

As used herein, the term “MCyR” means and is defined as the sum of overall CCR and the term “partial cytogenetic response” as used herein, means the condition in which Ph+ cells are less than or equal to 35% in bone marrow cells. See Bolton and Gathmann (2002), and O'Brien et al. N. Engl. J. Med., Vol. 348, pp. 994-1004 (2003). For this pharmacogenetic analysis, cytogenetic response was chosen as the primary efficacy criteria.

The 12-month data from CSTI571 0106 demonstrates that the MCyR rate (confirmed) in Imatinib-treated patients was 75.8%, compared to 12.1% for the combination of IFN-alpha and Ara-C. See Bolton and Gathmann (2002), supra, and O'Brien et al. N. Engi. J. Med., Vol. 348, pp. 994-1004 (2003). First-line treatment with Imatinib significantly increases the likelihood of achieving MCyR, a primary aim of CML treatment.

Gene expression variation in the response of CML patients to Imatinib has been examined prior to this report. A prediction score system has been published that has the ability to differentiate patients that responded to Imatinib compared to those that did not. The design of the prediction score system was based on the expression patterns of 15 or 30 genes. See Kaneta et al., Jpn. J. Cancer Res., Vol. 93, pp. 849-856 (2002).

In addition, several SNPs have been identified in the BCR-ABL kinase domain in patients whose disease relapses after an initial response to Imatinib. See Shah et al., Cancer Cell, Vol. 2, No. 2, pp. 117-125 (2002). These discoveries, along with the identification of the rs2290573 polymorphism as a genetic marker of response, could aid in elucidating which patients will respond to treatment with Imatinib.

This pharmacogenetic analysis of cytogenetic response in CSTI571 0106 was completed in order to address the secondary objective regarding the evaluation of RNA expression and DNA polymorphisms. A candidate gene approach was used to identify genetic markers of cytogenetic response in Imatinib-treated individuals.

Genotyping

SNPs were developed by two distinct methods. Third Wave Technologies, Inc. (Madison, Wis.) developed one collection of SNPs while the other set was developed in-house using a database mining approach. Public databases, such as OMIM, the SNP Consortium, Locus Link and dbSNP were utilized. Candidate genes were chosen based on rationale that included their involvement in edema, ADME, DNA repair, etiology of the disease or drug mechanism of action. Third Wave Technologies, Inc, developed the SNP assays for genotyping.

On the first day of study before treatment administration, 20 mL of blood was obtained from patients enrolled in the U.S. only. The baseline blood samples were collected after separate pharmacogenetics informed consent forms had been obtained according to protocols approved by local ethics committees. The DNA was extracted by the pharmacogenetics department using the PUREGENE” DNA Isolation Kit (D-50K) (Gentra, Minneapolis, Minn.) according to manufacturer's recommendations. See Pharmacogenetics, PGP-13 (2001). Genotyping was performed on 60 ng of genomic DNA using the Invader® assay (Third Wave Technologies, Inc.) according to the manufacturer's recommendations. See Lyamichev et al. (1999), supra.

A test for Hardy-Weinberg equilibrium (HWE) was performed as an additional quality control check. HWE was analyzed in the subset of genotyped individuals and in a subset of controls. The Hardy-Weinberg law states that allele frequencies do not change from generation to generation in a large population with random mating. Deviation from HWE would suggest one of two possibilities: 1) a genotyping error or 2) or an association between the polymorphism and the population being studied. In the second case, a particular polymorphism may be observed more frequently than would be expected if it is somehow involved in the disease etiology. All statistics were carried out in the statistical program SAS Version 8.2 (Cary, N.C.).

Statistical Methods

Representative Nature of the Genotyped Population

To determine how representative the genotyped population was of the entire CSTI571 0106 clinical trial population, the demographics of the two populations were compared. Furthermore, because the genotyped population consisted solely of U.S. patients, all U.S. patients in the CSTI571 0106 trial were also examined as a separate population. Age was compared using a non-parametric ANOVA and all other demographics were analyzed using exact tests in the statistical program SAS Version 8.2.

Correlation of Genotype with Response

An exact test was used to compare the genotype of each patient to OKR (best cytogenetic response, confirmed) stratified by treatment. Response status was determined from the 12-month eff1st.sd2 panel from the CSTI571 0106 clinical database created on Thursday, Apr. 11, 2002. This is the formal clinical database containing the data recorded for each patient during the study. A patients level of OKR is defined in the clinic by eight categories. These eight classes were divided into two distinct groups, MCyR and No MCyR. A patient was classified as achieving MCyR if their percentage of Ph+ cells was ≦35%. The non-responders included those patients with minor cytogenetic response, >35-65% Ph+cells, minimal cytogenetic response, >65-95% Ph+ cells and no cytogenetic response, >95-100% Ph+ cells, respectively (see Table 1). Efficacy was analyzed by first-line treatment results prior to crossover. Patients who discontinued treatment (n=14) or crossed to the alternative treatment arm (n=3) were not included in this portion of the pharmacogenetic analysis. Furthermore, patients classified as not assessable (NA) or progressive disease (PD) were not included in the analysis (n=7). Significant associations observed between OKR and a polymorphism were confirmed by analyzing the polymorphism with BKR (best cytogenetic response-unconfirmed). The number of patients utilized in the final pharmacogenetic analysis of first-line treatment was 109, including 91 Imatinib-treated patients. Twenty-nine (29) patients from the final group of 109 did not respond (OKR=3, 4 or 5), 18 of which were treated with Imatinib.

TABLE 1
Classifications of OKR Used for Pharmacogenetic Analysis

| MCyR | No MCyR |
|---|---|
| OKR = 1 (Complete, 0% Ph+ cells) | OKR = 3 (Minor, >35-65% Ph+ |
| | cells) |
| OKR = 2 (Partial, >0-35% Ph+ cells) | OKR = 4 (Minimal, >65-95% Ph+ |
| | cells) |
| | OKR = 5 (None, >95-100% Ph+ |
| | cells) |

Classifications of BKR are identical to those listed in Table 1.

The OR and 95% CIs were calculated by the Analyst Application, an interface for analysis in SAS Version 8.2. To correct for multiple testing a Bonferroni correction factor was applied.

Correlation Analysis Between Demographic, Genotypic and Phenotypic Variables

The sequencing of the human genome has generated much interest in biological differences among cultural populations. The array of genetic variation that exists across human populations becomes increasingly evident as genetics studies continue to uncover associations between biological findings and the racial and ethnic backgrounds of research participants. See Foster and Sharp, Genome Res., Vol. 12, No. 6, pp. 844-850 (2002). In order to assess this variance, the rs2290573 polymorphism by race was analyzed. In the CSTI571 0106 clinical trial data, race was classified as Caucasian, Black, Oriental and Other. To increase the statistical power, the analyses with race re-coded as Caucasian and Others was also performed.

Hasford and Sokal scores are used to predict survival and can also be used to select patients for different treatment. Sokal and Hasford predictive scores with response and the rs2290573 polymorphism in Imatinib-treated patients was analyzed. Anemia is a hematologic toxicity associated with the development of CML. The level of hemoglobin at each patients first visit (≦80, >80) with response in Imatinib-treated patients was analyzed. P-values were calculated using exact tests.

Logistic Repression Analysis

A logistic regression model was employed to investigate the association between MCyR and a set of explanatory variables. This analysis was designed to examine the cumulative predictability of genotype as a risk factor for response to Imatinib. The outcome variable of the logistic regression was MCyR, determined from the OKR column of the dataset (Table 1). A further analysis was completed with the outcome variable determined from the BKR column in the dataset.

Included in both models were genotype, race, hemoglobin levels (at first visit) and Sokal score.

Genotypes for the two polymorphisms that significantly associate with MCyR (OKR) by an exact test were incorporated in the multivariate analysis. These include the C-511T IL-1 beta polymorphism, coded as CC and CT+TT, and the rs2290573 polymorphism, coded as CC and CT+TT. Race was re-coded as two groups, Caucasian and Other, in order to increase statistical power.

Time to Progression

The relationship between genotype and MCyR was further investigated by analyzing time to progression (TTP). As used herein the term “TTP” is defined as the time between randomization and one of the following events:
1) Death (due to any cause when reported as primary reason for discontinuation of treatment);
2) Increase in WBC count (if approved by the Study Management Committee [SMC] as reason for crossover);
3) Loss of CHR;
4) Loss of MCyR; or
5) Progression to accelerated phase (AP) or blast crises (BC) (See Bolton and Gathmann (2002), supra, and O'Brien et al. N. Engl. J. Med., Vol. 348, pp. 994-1004 (2003)).

The product-limit method (Kaplan-Meier) was utilized to estimate the survival function directly from the continuous survival and failure times. The primary efficacy aim of the CSTI0106 trial was to determine whether Imatinib is superior to IFN-alpha+Ara-C in terms of TTP based on first-line treatment of patients with CML-CP utilizing the ITT principle. See Bolton and Gathmann (2002), supra, and O'Brien et al. N. Engl. J. Med., Vol. 348, pp. 994-1004 (2003).

For the pharmacogenetic analysis, only those patients randomized to Imatinib were included in the TTP analysis. There were only four events of progression in the 12-month data. Therefore, 18-month follow-up data was used in the TTP analysis with 10 events of progression. The analysis was stratified by genotype for the rs2290573 polymorphism to determine whether individuals with a CC genotype have a significantly higher proportion of progression events. TTP is censored at the date of last examination. Under the ITT principle the first evidence of progression is always considered even if there is another event that follows, e.g., loss of CHR followed by progression to AP/BC. See Bolton and Gathmann (2002), supra, and O'Brien et al. N. Engi. J. Med., Vol. 348, pp. 994-1004 (2003).

Results

Results of Correlation with Genotype and Response

Analysis of 68 genetic polymorphisms in 26 genes identified a significant association between the rs2290573 polymorphism and MCyR in Imatinib-treated patients using 12-month locked data, created on Thursday Apr. 11, 2002 (p=0.00036, Bonferroni corrected p=0.024).

In addition to analyzing the 12-month locked data by OKR, it was also analyzed by BKR. The rs2290573 polymorphism is also associated with BKR, p=0.019. The rs2290573 polymorphism lies within the intronic region of putative gene DKFZP434C131, on 15q22.33, and represents a C/T base transition. Imatinib-treated individuals with a CC genotype for this SNP have a response rate of 47%, while individuals with a CT or TT genotype have a response rate of 88% (see FIG. 4) (OR: 4.69, 95% CI: 1.24, 17.76). The significant association holds when 18-months of follow-up data is analyzed. The rs2290573 polymorphism was analyzed in 193 individuals from the STI571 0106 trial (Imatinib and IFN-alpha treated) and was found to be in HWE. Additionally, HWE was examined in 92 Caucasian and 73 African American controls.

The distribution of CC:CT:TT genotypes for the rs2290573 polymorphism in Caucasian controls was 45:34:13 and in African American controls was 43:18:5. Both sets of controls were found to be in HWE by Fisher's exact tests.

The association between the rs2290573 polymorphism and cytogenetic response was not observed in individuals treated with IFN-alpha. However, a significant association was also observed between the IL-1 beta C-511T polymorphism and OKR in Imatinib-treated patients (p=0.0431). The IL-1 beta C-511 T polymorphism was not associated with cytogenetic response in IFN-alpha Ara-C-treated patients (p>0.05).

Results of Correlation Between Demographic, Genotypic and Phenotypic Variables

The distribution of CC:CT+TT individuals for the rs2290573 polymorphism is significantly different between Caucasian (7:65), Black (10:1), Oriental (1:0) and Other (1:3). Because the number of Black, Oriental and Other individuals is small, individuals were re-categorized into two groups: Caucasian and Others. The genotype distribution for the rs2290573 polymorphism was significantly different between the two racial groups (p<0.001).

Due to the difference in genotype distribution for rs2290573 the correlation between genotype and OKR stratifying by race was analyzed. Race was categorized as Caucasians and Others. There was a significant association between the rs2290573 polymorphism and OKR in the Caucasian group treated with Imatinib (p=0.0125). In patients treated with Imatinib in the Other race category (Blacks, Orientals and others combined), a significant result was not observed between cytogenetic response and the polymorphism. The trend in the Caucasian group that Imatinib-treated individuals with a CC genotype have a greater chance of not responding than CT or TT individuals appears to be true in the “combined” racial category also, however, the small sample size is not powered enough to detect significance.

Due to the association between Sokal prognostic score and response to treatment with Imatinib, Sokal score with rs2290573 genotype was examined and observed a significant association (p<0.01). Using three categories of Sokal score (low-, intermediate- and high-risk) 40% of individuals with a CC genotype for the rs2290573 polymorphism had a high-risk Sokal score, while only 6.8% of individuals with CT and TT genotypes had a high-risk Sokal score. The high-risk Sokal score corresponds with the shortest survival time of 35 months. Hasford score does not significantly associate with the rs2290573 polymorphism. Hasford score takes eosinophils and basophils into account whereas Sokal score does not include these characteristics in the computational expression to derive a prognostic score.

Results of Logistic Repression

A multinomial logistic regression indicated that both the C-511T IL-1 beta polymorphism and the rs2290573 polymorphism are significant in the OKR model at the p=0.05 level of significance. A two-way interaction between these two significant effects did not yield a significant result.

Results of TTP

Survival analysis employing the ITT principle yielded 10 events in the Imatinib arm in the population of patients from the CSTI571 0106 trial that were genotyped in the 18-month data. Six of 26 Imatinib-treated patients (23.1%) with a CC genotype for the rs2290573 polymorphism experienced progression events. Four patients of 79 (5.1%) with a CT or TT genotype for the rs2290573 polymorphism experienced progression events (see Table 2). A significant difference was observed between genotypes according to the Log-Rank (0.0041) and Wilcoxon (0.0049) statistical tests. This observation strengthens the association reported between rs2290573 genotype and MCyR. The group of Imatinib-treated individuals with a CC genotype at this locus have a greater proportion of patients who experienced a progression event compared to the group of Imatinib individuals with a CT or TT genotype (see FIG. 4).

TABLE 2
Summary of TTP from 18-Month Data
(ITT principle/Imatinib-treated)

| | CC | CT, TT |
|---|---|---|
| | Genotype | Genotype |
| | n = 26 (%) | n = 79 (%) |
| Total number of patients with events | 6 (23.1) | 4 (5.1) |
| (progression) | | |
| Log-Rank test/Wilcoxon test | p < 0.01 | |
| Death (as primary reason for | 0 (0) | 0 (0) |
| discontinuation) | | |
| Progression to AP or BC | 0 (0) | 2 (2.5) |
| Loss of MCyR | 4 (15.4) | 0 (0) |
| Loss of CHR | 1 (3.8) | 2 (2.5) |
| Increase in WBC (approved by SMC) | 1 (3.8) | 0 (0) |

The primary objective for the CSTI571 0106 trial was to determine the time to progression in adult patients with newly diagnosed previously untreated Ph+ CML randomized to treatment with Imatinib compared to patients randomized to treatment with IFN-alpha+Ara-C. See Bolton and Gathmann (2002), supra, and O'Brien et al. N. Engl. J. Med., Vol. 348, pp. 994-1004 (2003). Secondary objectives included the determination of rate and duration of MCyR to Imatinib. MCyR is characterized by a presence of ≦35% Ph+cells.

The confirmed MCyR rate that was cited in the CSTI571 0106 Clinical Study Report for 12-month treatment with Imatinib was 75.8%. Bolton and Gathmann (2002), supra, and O'Brien et al. N. Engi. J. Med., Vol. 348, pp. 994-1004 (2003).

Pharmacogenetic Analysis

Pharmacogenetic analysis was performed on a subset of patients from the CSTI571 0106 trial to identify genetic markers of response to treatment with Imatinib. Statistical tests to identify any associations between polymorphisms in candidate genes and the presence or absence of MCyR in CML patients in chronic phase of disease treated with Imatinib for 12 months were performed. A significant association between the rs2290573 polymorphism mapped to the 15q22.33 region and the response classification of OKR was discovered. This association was also significant in the analysis of BKR. The analysis was completed with confirmed and unconfirmed cytogenetic response in order to align with the CSTI571 0106 Clinical Study Report. The genotype/phenotype association is striking because of the high response rate of CML patients in CSTI571 0106. The 47% of responders with a CC genotype at the putative gene locus considerably contrasts to the 75.8% of responders observed in the CSTI571 0106 Clinical Study Report. The association is also significant in data from 18-months of follow-up.

Multivariate analysis indicated that the rs2290573 polymorphism is a better predictor of cytogenetic response than Sokal score, race and hemoglobin levels in this population of CML patients.

Survival analysis of TTP illustrated a significant difference in progression events based on genotype for the rs2290573 polymorphism. A significantly greater percentage of patients with a CC genotype for the rs2290573 polymorphism experienced events of progression compared to patients with CT or TT genotype.

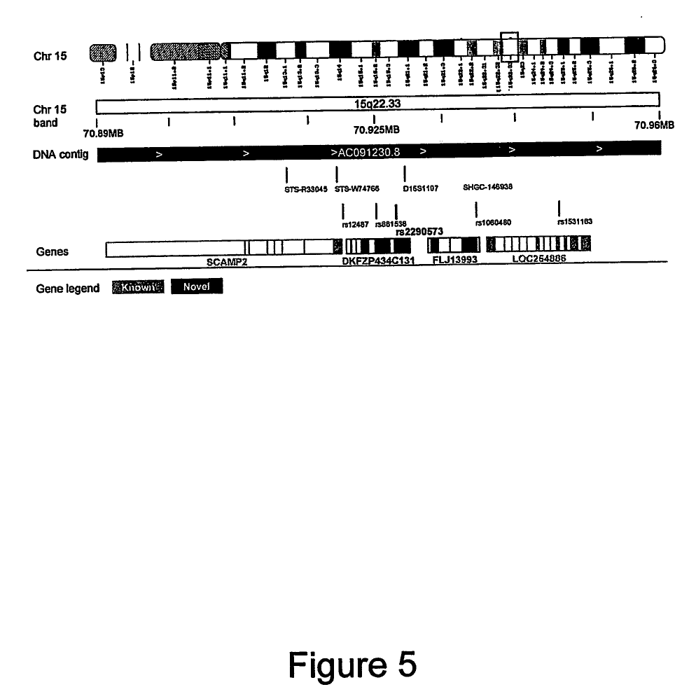

The rs2290573 polymorphism is currently mapped to a putative gene, DKFZP434C131, with a tyrosine kinase domain, near the SCAMP2 gene (see FIG. 5). This polymorphism was previously known as the polymorphism In the CSK gene at polymorphic site at position 36211 of sequence AC020705.4. At that time, the rs2290573 polymorphism reported here mapped to the CYP1A1 gene. Since then, the genetic map of the 15q region has been refined such that the polymorphism now maps to a putative gene, with the interim symbol of DKFZP434C131.

The genomic config NT_{—}010374.9, which was utilized to create the map of the region on chromosome 15, includes multiple breaks so that it is impossible at this time to determine how closely the polymorphism lies near CYP1A1. The region of the contig displayed in FIG. 5 is a continuous sequence and does not include any breaks.

Correction for Multiple Testing

Because of the nature of the approach used to identify predictive markers of edema it is necessary to correct for multiple testing. The more tests you perform the greater the chance of finding an association with p=0.05 by chance. There are several methods for correcting for multiple testing, none of which are ideal for pharmacogenetic analysis of large numbers of polymorphisms.

The Bonferoni correction for multiple testing is quite conservative and was developed at a time before it was possible to do testing on a genomic scale such is feasible now. To correct for multiple testing by using the Bonferoni correction factor the desired p-value is divided by the number of tests performed. The resulting value is the value that would be considered “significant”.

A second method to test the reliability of a dataset is bootstrapping. This method is a computer-intensive statistical analysis that applies simulation to calculate significance tests. Bootstrapping estimates the generalization error by creating a replicate of an entire dataset. A random number generator is utilized to resample the dataset. Bootstrapping was performed to test the stability of the significant results. The bootstrap consisted of two phenotypes (CHR and MCyR) and 69 SNPs and was run with 10,000 iterations.

All statistics were carried out in the statistical program SAS Version 8.2.

Results of Correlation with Genotype and Response

Calculation of Fishers Exact tests and odds ratios revealed three significant associations between genotype and hematological or cytogenetic response in STI571-treated patients, one in the CYP1A1 gene, the rs2290573 polymorphism (formely refered to as the CSK gene) and IL-1β gene. The 1462V polymorphism, consisting of a G→A nucleotide base change on exon 7 of the CYP1A1 gene, significantly correlates with CHR (p=0.004). The non-coding rs2290573 polymorphism significantly correlates with MCyR (p=0.004). The IL-1β −511 polymorphism found within the IL-1β promoter significantly correlates with MCyR (p=0.0121). The IL-1β polymorphism represents a C→T base transition at the position −511 base pairs from the transcriptional start site. The result of these associations is the identification of polymorphisms within three genetic loci which may predict the likelihood for CHR or MCyR, with an OR of 3.0 (95% CI: 1.2-7.4) or greater in patients treated with Imatinib.

TABLE 3
Association Results of the Variants (Analysis of Imatinib-Treated Patients Only)

| | | | | | | P- | |
|---|---|---|---|---|---|---|---|
| Gene | Location | Variant | Genotype | OR | 95% Cl | value | Response |
| rs2290573 | intron | — | CC/non-CC | 4.0 | 1.6-10.2 | 0.004 | MCyR |
| polymorph | | | | | | | |
| ism | | | | | | | |
| CYP1A1 | exon 7 | ILE462Val | AA/non-AA | 12.7 | 2.6-62.1 | 0.004 | CHR |
| IL-1β | promoter | −511 | CC/non-CC | 3.0 | 1.2-7.4 | 0.0121 | MCyR |

The IL-1beta polymorphism is a C → T at nucleotide position −511 (promoter region; no amino acid change); or C → T at nucleotide position 1423 of sequence X04500.
The CYP1A1 polymorphism is a G → A at position 6819 in sequence X02612 and causes an ILE to VAL at amino acid position 462 of the expressed protein.
The rs2290573 polymorphism, which is currently mapped to the putative gene, DKFZP434C131 in the 15q22.33 region, this putative gene codes for a tyrosine kinase (this polymorphism was formally mapped to the CYP1A1 gene and was referred to as the CSK gene and described as a C → T at position 36211 of sequence AC020705.4, with no amino acid change in the expressed protein).

CHR and MCyR are accurate indicators for the analysis of the efficacy of Imatinib mesylate (GLEEVEC® or GLIVEC® or STI571). Response, including cytogenetic and hematological, was the main focus for the analysis of efficacy in STI571 0106 clinical trial. Response consisted of a number of variables for first-line treatment, which is treatment before a patient crossed over from one drug to the other. This pharmacogenetic analysis was performed to identify genetic markers that could be used to predict the likelihood of response when treated with Imatinib.

Statistical tests to look for associations between genotypes in candidate genes and the presence or absence of response in Imatinib-treated patients, IFN-alpha+Ara-C-treated patients or in all patients, including both treatments were performed. Significant associations between three distinct genes were discovered: CYP1A1, the rs2290573 polymorphism, which is currently mapped to the putative gene, DKFZP434C131 in the 15q22.33 region, and IL-1 beta, and response classifications of CHR and MCyR, respectively.

### EXAMPLE 2

Pharmacogenomic Analysis of Cytogenetic Response in CML Patients Treated with Imatinib Mesylate (GLIVEC® or STI571)

Method Two

Gene Expression Profiling

In an investigation of cytogenetic response in CML patients treated with STI571, gene expression profiles from a total of 105 patients from the CSTI571 0106 Phase III clinical trial were studied. Gene expression data for more than 12,000 genes were generated from blood samples collected at baseline (prior to treatment with STI571) using A metrix oligonucleotide microarrays. Cytogenetic response was determined from 12-month data using the OKR data. To identify a group of genes that best differentiated between responders and non-responders, those individuals that experienced a CCyR (n=53) were compared with those that had minimal or NoCyR (n=13). An optimized set of 31 genes was identified by applying a “leave-one-out” analytical procedure. See van't Veer et al. (2002), supra. Using these genes, 94% of patients who achieved CCyR and 92% of those who had minimal or NoCyR were correctly identified. Results were also significant when those patients with partial or minor response were taken into consideration.

Pharmacogenomic analysis of RNA expression was conducted to identify genomic markers of cytogenetic response in the Phase III clinical trial CSTI571 0106. CSTI571 0106 was a study of STI571 vs. IFN-α combined with Ara-C in patients with newly-diagnosed, previously-untreated Ph+ CML-CP.

The primary objective of this study was to determine the time-to-treatment failure in patients randomized to STI571 compared to patients randomized to IFN-α+Ara-C. The secondary objectives included: 1) to determine the rate and duration of MCyR in patients randomized to STI571 compared to IFN-α+Ara-C; and 2) to perform pharmacogenomics evaluations to study In an exploratory fashion RNA expression and DNA polymorphisms, e.g., BCR-ABL and c-KIT, in tumor cells in the blood and bone marrow in this patient population.

This pharmacogenomic analysis was conducted in order to address the secondary objectives listed above. In an effort to develop predictive markers of cytogenetic response, RNA expression was evaluated to determine if there were differences in gene expression between those patients who achieved CCyR following treatment with STI571 compared to those that had minimal or NoCyR. In this study, RNA expression from blood collected at baseline prior to drug treatment was evaluated. Only those patients that were subsequently treated with STI571 as their first-line treatment were evaluated.

Cytogenetic response is defined in terms of the percentage of Ph+ metaphases in bone marrow cells. For this study, the cytogenetic response classification was taken from the OKR column in the derived response panel that looked at efficacy following first-line treatment from 12-month locked data (Jan. 31, 2002). Patients were classified as having CCyR if they had 0% Ph+ cells (OKR=1), and NoCyR if they had >65% Ph+ cells (OKR=4,5). Identifying a genomic profile of response is dependent upon a distinct phenotype. Consequently, patients with OKR scores of 2 (partial), 3 (minor), 6 (not assessable) or 7 (PD), or that had crossed-over or discontinued from the study were initially excluded. Therefore, the gene list was identified from the patients having the greatest phenotypic distinction between responders and non-responders.

Samples

Blood for RNA extraction was collected from more than 200 patients from the U.S. enrolled in the STI571 0106 clinical trial. Each of these patients signed a separate pharmacogenetic informed consent form that was approved by local IRB committees. A total of 115 samples were collected at baseline, prior to drug treatment, from patients that were randomized to the STI571 treatment arm as first-line treatment. Of these 115 samples, 9 were excluded from further analysis due to poor quality of processed RNA, and one sample was eliminated due to the patient dropping out very early from the study. Cytogenetic response (CyR) was determined from OKR of 12-month locked data (see Table 4). In order to create the largest distinction between responders and non-responders, the initial pharmacogenomic analysis was performed on a subset of the remaining 105 patients. Patients with 0% Ph+ cells (OKR=1) were classified as CCyR and those with >65% Ph+ cells (OKR=4 or OKR=5) were classified as NoCyR. Individuals that crossed over to the other treatment arm (n=1), or that were discontinued from the study (n=2), were not included in this initial analysis. This resulted in a total of 66 patient samples, 53 with CCyR and 13 with minimal or NoCyR.

RNA Expression Profiling

Total RNA was isolated from whole blood, processed and hybridized to Affymetrix U95A, version 2, microarrays by the Pharmacogenetics group in Gaithersburg, Md. using methods using TRI REAGENT™ BD described else where in this specification.

Analytical Strategy

To determine the best set of predictor genes from the 12,627 probe sets on the Affymetrix U95A microarrays, the gene list was first filtered and the “leave-one-out” analytical strategy described recently in van't Veer et al. (2002), Nature; 415: 530-536 was adapted, to determine the optimal number of genes to use for the genomic profile.

Filtering of Data

All data for the 66 samples were directly imported into GENESPRING® Version 4.2.1 (Silicon Genetics, Redwood City, Calif.). The default GENESPRING® normalization was chosen: the 50 percentile of all measurements was used as a positive control for each sample; each measurement for each gene was divided by this synthetic positive control, assuming that this was at least 10. The bottom tenth percentile was used as a test for correct background subtraction. This was never less than the negative of the synthetic positive control. Each gene was normalized to itself by making a synthetic positive control for that gene, and dividing all measurements for that gene by this positive control, assuming it was at least 0.01. This synthetic control was the median of the gene's expression values over all the samples.

Filtering of the data was performed to remove probe sets that showed little or no expression across all of the samples. Raw expression values were filtered such that at least 10% of the samples (7 of 66) had an AvgDiff value of 100 or greater above background. Probe sets were further filtered out if there was no significant difference between the CCyR and NoCyR groups. This was done using the non-parametric (Wilcoxon-Mann-Whitney) test on the normalized data with a p-value cutoff of 0.05. This filtering resulted in a total of 300 probe sets out of the original 12,627. The raw data for these 300 probe sets (minus 2 Affymetrix control probe sets) was exported to Excel and transformed such that all negative AvgDiff values were set equal to 1. Additional probe sets were filtered out if there were less than 5% present (P) or marginal (M) calls, and if there were more than 50% negative values in both groups. Mean AvgDiff values were calculated for the two groups and the ratio of CCyR to NoCyR determined. Probe sets that had a fold difference of less than 1.7 were excluded from further analysis, leaving a total of 71 probe sets. Data for these 71 probe sets was exported into SAS version 8.2 and a non-parametric, one-way ANOVA performed between the CCyR and NoCyR groups. A total of 55 probe sets were significantly different between the two groups with a p-value <0.05.

Determination of Optimum Number of Reporter Genes

The “leave-one-our procedure was used to determine the optimum number of genes, from the 55 genes that could distinguish between the CCyR and NoCyR. The analysis was essentially identical to that described in the supplemental Information of the van't Veer et al. (2002), supra, paper and is explained below. First, the correlation between gene expression (with negative raw values set=1) and the prognostic category (NoCyR=0; CCyR=1) was calculated for each of the 55 genes across all of the 66 samples. The absolute value of the Pearson correlation coefficient was taken for each gene (so that equal weight was given to positive and negative correlations), and the genes were then ordered from highest to lowest correlation.

Starting with the top 5 genes (those with the highest correlation to CCyR prognosis), one sample was taken out of the analysis and the mean gene expression profile for each group (CCyR and NoCyR) was calculated from the remaining 65 samples. Then the predicted outcome for the left-out sample was calculated by determining the PCC of the expression profile of the left out sample with the mean CCyR and NoCyR profiles calculated using the 65 samples. If the CC was higher from the CCyR correlation than the NoCyR, the sample was classified as CCyR and assigned a value of 1. This analysis was repeated using the remaining samples until all 66 samples had been left out once. The number of cases of correct and incorrect predictions was then determined by calculating the number of false negatives (CCyR misclassified as NoCyR) and false positives (NoCyR misclassified as CCyR). The entire “leave-one-out” process was repeated after adding additional genes, from the top of the list, until all 55 genes were used. The error rates as function of number of genes are presented in FIG. 1.

Determination of Optimal Threshold Value

Using these optimal 31 genes (see Results for listing of genes), the next step was to calculate the appropriate threshold value to use for an accurate distinction between CCyR and NoCyR. It was empirically decided to compare individual samples to the mean NoCyR profile as opposed to the CCyR profile after comparing results from both (clustering results were substantially better using the NoCyR profile). A PCC was used to compare the expression pattern of the 31 genes for each of the 66 samples to the mean NoCyR profile (calculated using the 13 of the 66 patients with NoCyR). Patient samples were then ranked by correlation from highest to lowest and error rates were determined as a function of where the threshold correlation was drawn. The results are displayed in FIG. 2. The threshold at “optimal accuracy” was at a CC (r) of 0.570, which was the point where there was the minimum of both false positives (N=2) and false negatives (N=2).

In an effort to further reduce the number of false positives (NoCyR misclassified as CCyR), a second threshold was selected at r=0.540 (optimized specificity). Using this threshold, the number of false positives (N=1) was reduced, while only slightly increasing the number of false negatives (N=3). Using a threshold at r=0.437 would further increase the specificity by eliminating all false positives, though this would result in a large increase in false negatives (N=11) and thus diminished sensitivity. Based on these results, it was decided to use a threshold at r=0.54 for all subsequent analyses.

Statistical Analyses

Statistical analyses such as Fishers Exact test, non-parametric one-way ANOVA, and calculation of ORs, along with 95% CIs, were calculated using SAS version 8.2. Statistical significance was established at p<0.05.

Classification of Cytogenetic Response

As indicated in Table 4, the distribution of OKR for the subset of 105 patients was comparable to all of the U.S. patients in the STI571 arm (from which the 105 were taken), as well as to the entire STI571-treated patient population for this trial.

TABLE 4
Distribution and Frequencies of Cytogenetic Response

| | | | Patients | U.S. STI571 | All STI571 |
|---|---|---|---|---|---|
| | | % Ph+ | Analyzed* | Patients | Patients |
| OKR | Description | Cells | N (%) | N (%) | N (%) |
| 1 | Complete | 0 | 54 (51.4) | 112 (52.1) | 297 (53.7) |
| 2 | Partial | >0-35 | 22 (21.0) | 53 (24.7) | 122 (22.1) |
| 3 | Minor | >35-65 | 7 (6.7) | 10 (4.7) | 21 (3.8) |
| 4 | Minimal | >65-95 | 4 (3.8) | 8 (3.7) | 18 (3.3) |
| 5 | None | >95 | 11 (10.5) | 19 (8.8) | 66 (11.9) |
| 6 | Not | — | 5 (4.8) | 9 (4.2) | 19 (3.4) |
| | Accessible | | | | |
| 7 | Progressive | — | 2 (1.9) | 4 (1.9) | 7 (1.3) |
| | Disease | | | | |
| 8 | Death | — | 0 (0.0) | 0 (0.0) | 3 (0.5) |
| | | Total | 105 (100.0) | 215 (100.0) | 553 (100.0) |

*Patients analyzed were a subset of U.S. patients enrolled in the IRIS Study that were randomized to the STI571 treatment arm

Quality Control of Microarrays

The mean scaling factor for the 115 baseline patient samples in the STI571 treatment arm was 55.6±86.2 (StdDev) with a range from 4.7-644.7. A total of 9 samples had scaling factors greater than 141.8 (mean+1 StdDev), which were considered to be highly unreliable, and were thus excluded from all analyses. As demonstrated in Table 5, there were no statistically significant differences in quality control parameters between the CCyR and NoCyR groups for the 66 samples used in the analysis to identify the genomic markers of response.

TABLE 5
Summary of Quality Control Factors

| | | | GAPDH: | β-actin: |
|---|---|---|---|---|
| | Scaling | % Genes | 3′/5′ | 3′/5′ |
| | Factor | Present | ratio | ratio |
| Minimum | | | | |
| CcyR | 6.2 | 5.2 | 1.0 | 0.6 |
| NoCyR | 7.0 | 5.6 | 1.6 | 2.6 |
| Maximum | | | | |
| CCyR | 137.8 | 31.3 | 18.8 | 30.2 |
| NoCyR | 101.7 | 23.7 | 25.7 | 23.7 |
| Mean | | | | |
| CCyR | 36.2 | 13.3 | 6.1 | 8.4 |
| NoCyR | 37.8 | 14.0 | 6.7 | 9.3 |
| Median | | | | |
| CCyR | 28.4 | 11.2 | 5.3 | 6.6 |
| NoCyR | 20.0 | 11.9 | 4.7 | 7.3 |
| Std | | | | |
| Deviation | | | | |
| CCyR | 29.6 | 6.7 | 4.1 | 6.4 |
| NoCyR | 32.5 | 6.9 | 6.3 | 6.4 |
| Std Error | | | | |
| CCyR | 4.1 | 0.9 | 0.6 | 0.9 |
| NoCyR | 9.0 | 1.9 | 1.7 | 1.8 |
| T-test | | | | |
| p-value | | | | |
| CCyR vs. | 0.8683 | 0.7656 | 0.6777 | 0.6822 |
| NoCyR | | | | |

CCyR = complete cytogenetic response (OKR = 1).
NoCyR = minimal or no response (OKR = 4, 5).

The 31 Genes Comprising the Genomic Profile

Selection of the 31 genes comprising the genomic profile of cytogenetic response was performed using a set of 66 samples (53 CCyR, 13 NoCyR) as outlined in detail in the Methods section. Table 6 presents a list of these genes along with their Affymetrix probe set name, GenBank accession number, chromosomal locus and a brief description of function.

Details, such as mean expression values, standard error (SE), fold difference between CCyR and NoCyR groups, as well as the PCC (r) of each gene to response status and results of non-parametric, one-way ANOVA comparing CCyR to NoCyR, are presented in Tables 12A and 12B for all 55 genes that were found to discriminate between responders and non-responders.

Interestingly, many of the genes identified in this report as having different expression between responders and non-responders appear to be functionally related to the pathogenesis of CML via the BCR-ABL oncogene, including altered cell adhesion; constitutively active mitogenic signaling; and inhibition of apoptosis. See Faderl et al., N. Engl. J. Med., Vol. 341, No. 3, pp. 164-172 (1999); Deininger et al., Blood, Vol. 96, No. 10, pp. 3343-3356 (2000); and Kabaraowski and Witte, Stem Cells, Vol. 18, No. 6, pp. 399-408 (2000), for reviews.

TABLE 6
Set of 31 Genes Comprising Genomic Profile of Cytogenetic Response

| | | | | | General |
|---|---|---|---|---|---|
| Probe Set | Accession | Gene | Locus | Description | Function |
| 40793_s_at | U34846 | AQP4 | 18q11.2-q12.1 | Aquaporin 4 | Water transport |
| 34416_at | X57110 | CBL | 11q23.3 | Cas-Br-M (murine) ecotropic | Signal |
| | | | | retroviral transforming | transduction |
| | | | | sequence | |
| 37535_at | M27691 | CREB1 | 2q32.3-q34 | cAMP responsive element | Transcription |
| | | | | binding protein 1 | factor |
| 33949_at | AF011406 | CRHR2 | 7p15.1 | Corticotropin releasing | Signal |
| | | | | hormone receptor 2 | transduction |
| 34077_at | X95876 | CXCR3 | Xq13 | Chemokine (C-X-C motif) | Cell adhesion |
| | | | | receptor 3 | |
| 1305_s_at | D12620 | CYP4F3 | 19p13.2 | Cytochrome P450, subfamily | Metabolism |
| | | | | IVF, polypeptide 3 (leukotriene | |
| | | | | B4 omega hydroxylase) | |
| 39692_at | AL080209 | DKFZp586F2423 | 7q34 | Hypothetical protein | Unknown |
| | | | | DKFZp586F2423 | |
| 38226_at | W27152 | FLJ10569 | 8p21.2 | Hypothetical protein FLJ10569 | Unknown |
| 36120_at | X63657 | FVT1 | 18q21.3 | Follicular lymphoma variant | Oncogenesis |
| | | | | translocation 1 | |
| 36741_at | D63482 | GIT2 | 12q24.1 | G protein-coupled receptor | Signal |
| | | | | kinase-interactor 2 | transduction |
| 38986_at | Z49835 | GRP58 | 15q15 | Glucose regulated protein, | Signal |
| | | | | 58 kD | transduction |
| 31919_at | AF002986 | H963 | 3q26.1 | Platelet activating receptor | Immune |
| | | | | homolog | response |
| 38467_at | U96721 | HPS1 | 10q23.1-q23.3 | Hermansky-Pudlak syndrome | Organelle |
| | | | | | function |
| 1457_at | M64174 | JAK1 | 1p32.3-p31.3 | Janus kinase 1 (a protein | Signal |
| | | | | tyrosine kinase) | transduction |
| 41614 at | AB014608 | KIAA0708 | 6p12.3 | KIAA0708 protein | Unknown |
| 37542_at | D86961 | LHFPL2 | 5q13.3 | Lipoma HMGIC fusion partner- | Unknown |
| | | | | like 2 | |
| 35180_at | AL050205 | LOC113251 | 12q13.12- | c-MpI binding protein | Unknown |
| | | | q13.13 | | |
| 32847_at | U48959 | MYLK | 3q21 | Myosin, light polypeptide | Signal |
| | | | | kinase | transduction |
| 1558_g_at | U24152 | PAK1 | 11q13-q14 | p21/Cdc42/Rac1-activated | Signal |
| | | | | kinase 1 (STE20 homolog, | transduction, |
| | | | | yeast) | cytoskeleton |
| 1269_at | M61906 | PIK3R1 | 5q12-q13 | Phosphoinositide-3-kinase, | Signal |
| | | | | regulatory subunit, polypeptide | transduction |
| | | | | 1 (p85 alpha) | |
| 34376_at | AB019517 | PKIG | 20q12-q13.1 | Protein kinase (cAMP- | Signal |
| | | | | dependent, catalytic) inhibitor | transduction |
| | | | | gamma | |
| 623_s_at | M28213 | RAB2 | 8q12.1 | RAB2, member RAS oncogene | Protein |
| | | | | family | transport |
| 32158 at | U53174 | RAD9 | 11q13.1-q13.2 | RAD9 homolog (S. pombe) | DNA repair |
| 32849_at | D80000 | SMC1L1 | Xp11.22- | SMC1 structural maintenance | Chromosome |
| | | | p11.21 | of chromosomes 1-like 1 | structure |
| | | | | (yeast) | |
| 351_f_at | D28423 | SFRS3 | 11 | Splicing factor, arginine/serine- | RNA binding |
| | | | | rich 3, 5′UTR (sequence from | |
| | | | | 5′cap to start codon) | |
| 37050_r_at | AI130910 | TOMM34 | 20 | Translocase of outer | Protein |
| | | | | mitochondrial membrane 34 | transport |
| 39341_at | AJ001902 | TRIP6 | 7q22 | Thyroid hormone receptor | Transcription |
| | | | | interactor 6 | factor |
| 33346_r_at | M61764 | TUBG1 | 17q21 | Tubulin, gamma 1 | Cytoskeleton |
| 39867_at | S75463 | TUFM | 16p11.2 | Tu translation elongation factor, | Protein |
| | | | | mitochondrial | synthesis |
| 32518_at | AF019767 | ZNF259 | 11q23.3 | Zinc finger protein 259 | Signal |
| | | | | | transduction |
| 36303_f_at | U35376 | ZNF85 | 19p13.1-p12 | Zinc finger protein 85 (HPF4, | Transcription |
| | | | | HTF1) | factor |

Genes determined from “leave-one-out” analysis of 55 potential candidate genes using 66 patient samples (53 with CCyR and 13 with NoCyR).

Classification of Response Status Using the 31-Gene Genomic Profile

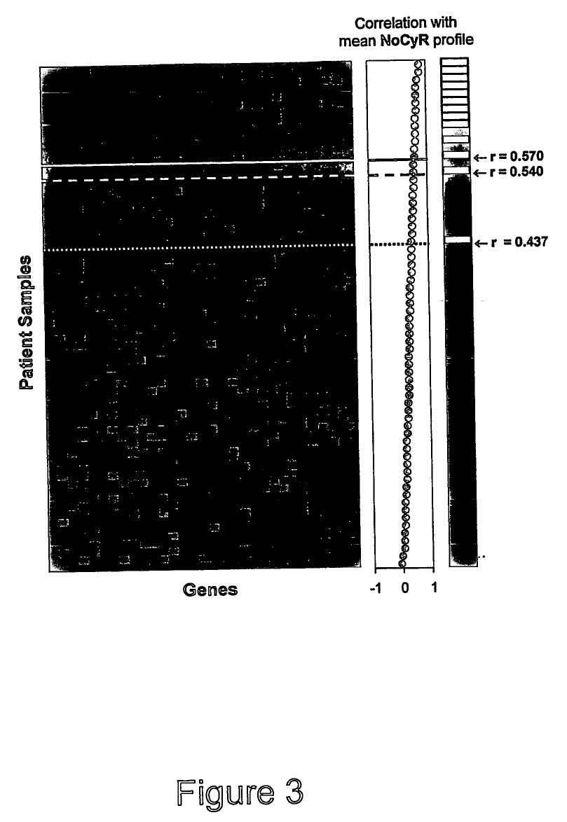

FIG. 3 displays the results of cluster analysis of the 31 genes that comprise the cytogenetic response profile, with the samples ordered by CC. Samples with the highest correlation with the NoCyR profile are at the top, and those with least correlation with NoCyR status are at the bottom. A threshold correlation was selected at a value that would minimize the number of false negatives to less than 10% (optimized specificity; r=0.54). Using this threshold, an individual with a CC of ≦0.54 (based on PCC with mean NoCyR expression profile) would be classified in the NoCyR group, while an individual with r<0.54 would be classified in the CCyR group. Table 7 displays the results of the frequency analysis, indicating the number of correct and incorrect predictions, using this threshold value. The difference between the actual response data and the calculated response data (based upon the genomic profile) was highly significant according to a Fisher's exact test, with a p-value of 5.29×10⁻⁹.

TABLE 7
Frequency Analysis and Calculation of ORs for CCyR (OKR-1 vs. OKR-4, 5)

| | | Observed (Expected) | | | | | | | |
|---|---|---|---|---|---|---|---|---|---|
| | | | No | | | | | | |
| | | Response | Response | | | | | | |
| Response | | Profile | Profile | OR | | | | | |
| Status | N | (r < Thr) | (r ≧ Thr) | (95% CI) | p-value | Sens. | Spec. | PV+ | PV− |

Threshold = 0.540 (optimized specificity: <10% false negatives)

| | | | | | | | | | |
|---|---|---|---|---|---|---|---|---|---|
| CCyR | 53 | 50 (41) | 3 (12) | 200 | 1.14E−09 | 0.943 | 0.923 | 0.980 | 0.800 |
| | | | | (19.1-2096) | | | | | |
| NoCyR | 13 | 1 (10) | 12 (3) | | | | | | |

Threshold = 0.437 (optimized specificity: 0 false negatives)

| | | | | | | | | | |
|---|---|---|---|---|---|---|---|---|---|
| CCyR | 53 | 42 (34) | 11 (19) | 99.8 | 1.22E−07 | 0.792 | 1.000 | 1.000 | 0.542 |
| | | | | (5.5-1807) | | | | | |
| NoCyR | 13 | 0 (8) | 13 (5) | | | | | | |

r = PCC,
Thr = threshold correlation value,
CI: confidence interval,
Sens. = sensitivity,
Spec. = Specificity
PV+ = predictive value positive,
PV− = predictive value negative
CCyR = complete cytogenetic response, confirmed (OKR = 1);
NoCyR = minimal (OKR = 4) or no (OKR = 5)

The OR in this case indicates that an individual with an expression profile for the 31 genes that is closely correlated with the mean NoCyR profile (r≧0.54) is approximately 200 times more likely to not achieve complete cytogenetic response compared to an individual with r<0.54. Using this threshold value, values for sensitivity and specificity of 0.943 and 0.923, respectively were achieved (see Table 7). To increase the specificity to a value of 1.00 (no cases of NoCyR misclassified), a threshold value of 0.437 would be required (see FIG. 3). This results in a decrease of sensitivity to 0.79, although the OR of 99.8 (95% CI: 5.5-1807) is still highly-significant (see Table 7).

To determine the optimum set of genes that could differentiate between responders and non-responders, only those patients who achieved CCyR (OKR=1) and those with NoCyR (OKR=4,5) were used, with the exclusion of 3 individuals who had crossed over or had been discontinued. Subsequent analysis was performed to evaluate the calculated response for all of the 105 individuals in this study using such genomic profile. For these analyses, the profile of the 31 genes for each of the samples was correlated against the mean NoCyR profile using the threshold correlation value of 0.54 (see FIG. 3 and Table 7).

Table 8 displays the breakdown of genomic classification by reported best cytogenetic response, as defined in Table 4, using 12-month data. This was done using both the OKR and BKR data. Results of analysis by Fisher's exact test indicated that there was a significant association between the response calculated by genomic profiling and the actual best cytogenetic response (p<0.000001) for both the OKR and BKR data. Table 9 shows the results of follow-up analysis using the 18-month clinical data, and indicates that the association between calculated response and actual cytogenetic response remains significant (p<0.00001).

TABLE 8
Genomic Classification of Cytogenetic Response (r = 0.54)
vs. Actual Best Cytogenetic Response (12-month data)

| | | Using OKR Data | | | | | Using BKR Data | | | | |
|---|---|---|---|---|---|---|---|---|---|---|---|
| | | Response | | No Response | | | Response | | No Response | | |
| Best | | Profile | | Profile | | | Profile | | Profile | | |
| Cytogenetic | | (r < 0.54) | | (r ≧ 0.54) | | | (r < 0.54) | | (r ≧ O.54) | | |
| Response | | Obs | (Exp) | Obs | (Exp) | Total | Obs | (Exp) | Obs | (Exp) | Total |
| 1. | Complete | 51 | (44) | 3 | (10) | 54 | 65 | (56) | 4 | (13) | 69 |
| 2. | Partial | 20 | (18) | 2 | (4) | 22 | 13 | (14) | 4 | (3) | 17 |
| 3. | Minor | 6 | (6) | 1 | (1) | 7 | 2 | (2) | 1 | (1) | 3 |
| 4. | Minimal | 0 | (3) | 4 | (1) | 4 | 4 | (6) | 3 | (1) | 7 |
| 5. | None | 2 | (9) | 9 | (2) | 11 | 0 | (6) | 7 | (1) | 7 |
| 6. | Not assessible | 5 | (4) | 0 | (1) | 5 | 0 | (0) | 0 | (0) | 0 |
| 7. | Progressive | 2 | (2) | 0 | (0) | 2 | 2 | (2) | 0 | (0) | 2 |
| | disease | | | | | | | | | | |

| | | | | | | | | | | |
|---|---|---|---|---|---|---|---|---|---|---|
| TOTAL | 86 | (86) | 19 | (19) | 105 | 86 | (86) | 19 | (19) | 105 |

| | |
|---|---|
| Fisher's exact: p = 1.08 × 10⁻⁸ | Fisher's exact: p = 1.77 × 10⁻⁷ |

r = PCC

TABLE 9
Genomic Classification of Cytogenetic Response (r = 0.54)
vs. Actual Best Cytogenetic Response (18-month data)

| | | Using OKR Data | | | | | Using BKR Data | | | | |
|---|---|---|---|---|---|---|---|---|---|---|---|
| | | Response | | No Response | | | Response | | No Response | | |
| Best | | Profile | | Profile | | | Profile | | Profile | | |
| Cytogenetic | | (r < 0.54) | | (r ≧ 0.54) | | | (r < 0.54) | | (r ≧ O.54) | | |
| Response | | Obs | (Exp) | Obs | (Exp) | Total | Obs | (Exp) | Obs | (Exp) | Total |
| 1. | Complete | 57 | (50) | 4 | (11) | 61 | 67 | (59) | 5 | (13) | 72 |
| 2. | Partial | 15 | (16) | 4 | (3) | 19 | 11 | (13) | 5 | (3) | 16 |
| 3. | Minor | 5 | (6) | 2 | (1) | 7 | 2 | (2) | 1 | (1) | 3 |
| 4. | Minimal | 0 | (1) | 1 | (0) | 1 | 4 | (5) | 2 | (1) | 6 |
| 5. | None | 1 | (7) | 8 | (2) | 9 | 0 | (5) | 6 | (1) | 6 |
| 6. | Not assessible | 5 | (4) | 0 | (1) | 5 | 0 | (0) | 0 | (0) | 0 |
| 7. | Progressive | 3 | (2) | 0 | (1) | 3 | 2 | (2) | 0 | (0) | 2 |
| | disease | | | | | | | | | | |

| | | | | | | | | | | |
|---|---|---|---|---|---|---|---|---|---|---|
| TOTAL | 86 | (86) | 19 | (19) | 105 | 86 | (86) | 19 | (19) | 105 |

| | |
|---|---|
| Fisher's exact: p = 1.32 × 10⁻⁸ | Fisher's exact: p = 1.97 × 10⁻⁷ |

r = PCC

Additional analysis was performed by grouping individuals into 2 categories based upon their actual best cytogenetic response. Individuals with 0-35% Ph+ cells (complete+partial response) were classified as having achieved MCyR or MKR, based upon whether the classification was made using OKR data or BKR data, respectively. Those individuals that had >35% Ph+ cells (minor, minimal and no response) were classified as non-responders (NoMCyR or NoMKR). Patients with OKR or BKR equal to 7 (progressive disease) were included in the non-responder category, while patients with OKR or BKR equal to 6 (not assessable) were excluded from these analyses. Results of these analyses, for both the 12-month and 18-month data, are included in Table 10. In all cases, the results were highly-significant (p<0.001), with ORs ranging from 8.8 (95% CI: 2.8-27.9) to 19.9 (95% CI: 5.9-67.1). Sensitivity ranged from 0.886-0.934, while specificity only ranged from 0.529-0.583.

TABLE 10
Frequency Analysis and Calculation of ORs for MCyR and MKR

| | | Observed (Expected) | | | | | | | |
|---|---|---|---|---|---|---|---|---|---|
| | | Response | No Response | | | | | | |
| Response | | Profile | Profile | OR | | | | | |
| Status | N | (r < 0.54) | (r ≧ 0.54) | (95% CI) | p-value | Sens. | Spec. | PV+ | PV− |

Using 12-month clinical data:

MCyR (OKR-1, 2 vs. OKR-3, 4, 5, 7)

| | | | | | | | | | |
|---|---|---|---|---|---|---|---|---|---|
| MCyR | 76 | 71 (62) | 5 (14) | 19.9 | 2.9 × 10⁻⁷ | 0.934 | 0.583 | 0.877 | 0.737 |
| | | | | (5.9-67.1) | | | | | |
| NoMCyR | 24 | 10 (19) | 14 (5) | | | | | | |

MKR (BKR-1, 2 vs. BKR-3, 4, 5, 7)

| | | | | | | | | | |
|---|---|---|---|---|---|---|---|---|---|
| MKR | 86 | 78 (70) | 8 (16) | 13.4 | 1.2 × 10⁻⁵ | 0.907 | 0.579 | 0.907 | 0.579 |
| | | | | (4.2-43.0) | | | | | |
| NoMKR | 19 | 8 (16) | 11 (3) | | | | | | |

MCyR (OKR-1, 2 vs. OKR-3, 4, 5, 7)

| | | | | | | | | | |
|---|---|---|---|---|---|---|---|---|---|
| CCyR | 80 | 72 (65) | 8 (15) | 11.0 | 4.6 × 10⁻⁵ | 0.900 | 0.550 | 0.889 | 0.579 |
| | | | | (3.5-34.5) | | | | | |
| NoCyR | 20 | 9 (16) | 11 (4) | | | | | | |

MKR (BKR-1, 2 vs. BKR-3, 4, 5, 7)

| | | | | | | | | | |
|---|---|---|---|---|---|---|---|---|---|
| MKR | 88 | 78 (72) | 10 (16) | 8.8 | 0.0003 | 0.886 | 0.529 | 0.907 | 0.474 |
| | | | | (2.8-27.9) | | | | | |
| NoMKR | 17 | 8 (14) | 9 (3) | | | | | | |

r = PCC,;
CI = confidence interval;
Sens. = sensitivity;
Spec. = Specificity;
PV+ = predictive value positive;
PV− = predictive value negative;
MCyR = major cytogenetic response, confirmed;
NoMyR = no major confirmed response;
MKR = major cytogenetic response, unconfirmed;
NoMKR = no major response, unconfirmed;
OR = Odds Ratio.

Correlation of Cytogenetic Response with TTP

The primary objective of the IRIS study was to determine the TTP of Ph+ CML patients randomized to treatment with STI571 compared to patients randomized to treatment with IFN-alpha+Ara-C. Looking at the 12-month data, there were 24 individuals out of the entire 553 Imatinib-treated patients who exhibited disease progression. Three of these individuals were included in the subset of 105 patients for which expression data were available.

TTP was evaluated using the 18-month clinical data. An additional 18 individuals from the entire STI571 treatment arm had disease progression (TTP_C=0), 7 of which were included in our 105-patient population for genomic analysis. Table 11 displays details of response data for the 10 patients with disease progression in our analysis population of 105 patients. Nine out of the 10 individuals that experienced disease progression had an expression profile that more closely correlated with the response profile (r<0.54). Furthermore, several of these individuals did experience improvement following STI571 treatment, as indicated by their values for both BKR and OKR (see Table 11).

TABLE 11
Response Data for Individuals with Disease Progression (TTP_C = 0)
from the 105 Analyzed STI571-Treated Patients (18-month data)

| | | Genomic | | | | | | | TTP | |
|---|---|---|---|---|---|---|---|---|---|---|
| Patient | r | Profile | OKR | | MCyR | BKR | | MKR | (months) | TTPR |
| 0756_00007 | 0.285 | CCyR | 7 | (progressive | 0 | 7 | (progressive | 0 | 6.8 | 2 |
| | | | | disease) | | | disease) | | | (progress |
| | | | | | | | | | | to AP/BC) |
| 0744_00001 | 0.262 | CCyR | 3 | (minor) | 0 | 3 | (minor) | 0 | 16.1 | 3 |
| | | | | | | | | | | (loss of |
| | | | | | | | | | | CHR) |
| 0757_00046 | 0.467 | CCyR | 3 | (minor) | 0 | 2 | (partial) | 1 | 16.3 | 3 |
| | | | | | | | | | | (loss of |
| | | | | | | | | | | CHR) |
| 0738_00004 | 0.358 | CCyR | 2 | (partial) | 1 | 1 | (complete) | 1 | 11.8 | 4 |
| | | | | | | | | | | (loss of |
| | | | | | | | | | | MKR) |
| 0714_00010 | 0.448 | CCyR | 3 | (minor) | 0 | 2 | (partial) | 1 | 17.0 | 4 |
| | | | | | | | | | | (loss of |
| | | | | | | | | | | MKR) |
| 0771_00013 | 0.609 | NoCyR | 3 | (minor) | 0 | 2 | (partial) | 1 | 13.4 | 4 |
| | | | | | | | | | | (loss of |
| | | | | | | | | | | MKR) |
| 0744_00002 | 0.359 | CCyR | 1 | (complete) | 1 | 1 | (complete) | 1 | 16.4 | 5 |
| | | | | | | | | | | (increase |
| | | | | | | | | | | in WBC) |
| 0737_00002 | 0.498 | CCyR | 3 | (minor) | 0 | 3 | (minor) | 0 | 9.7 | 5 |
| | | | | | | | | | | (increase |
| | | | | | | | | | | in WBC) |
| 0724_00004 | 0.437 | CCyR | 7 | (progressive | 0 | 4 | (minimal) | 0 | 15.8 | 5 |
| | | | | disease) | | | | | | (increase |
| | | | | | | | | | | in WBC) |
| 0762_00003 | 0.137 | CCyR | 7 | (progressive | 0 | 7 | (progressive | 0 | 5.6 | 5 |
| | | | | disease) | | | disease) | | | (increase |
| | | | | | | | | | | in WBC) |

r = PCC (compared to mean NoCyR profile),
CCyR = responder (r < 0.54),
NoCyR = non-responder (r ≧ 0.54),
OKR = best cytogenetic response, confirmed;
MCyR = major cytogenetic response, confirmed (OKR = 1, 2);
BKR = best cytogenetic response, unconfirmed;
MKR = major cytogenetic response, unconfirmed (BKR = 1, 2);
TTP = time to progression, months;
TTPR = reason for progression;
AP = accelerated phase;
BC = blast crisis;
WBC = white blood cells;
PD = progressive disease

This pharmacogenomic analysis was performed to identify genomic markers of cytogenetic response following treatment with STI571 (see Table 13 for definitions of cytogenetic response). Utilizing the analytical strategy described by van't Veer et al. (2002), supra, an optimal set of 31 genes for which a patient's expression profile correlated with CCyR following treatment with STI571 was defined. This was performed using a subset of the patients that exhibited the greatest phenotypic distinction, those with CCyR vs. NoCyR. Statistical analyses were performed using a PCC (r) that was optimized for specificity such that no more than 10% of non-responders would be misclassified as responders (r=0.54; see FIG. 3). This resulted in a sensitivity of 0.943 and specificity of 0.923, with an OR of 200 (95% CI: 19.1-2096) and p<0.0001 (see Table 7). The OR in this case indicating that an individual has an approximately 200-fold greater probability of not achieving CCyR, following treatment with STI571, if their correlation to the mean NoCyR expression profile resulted in r≧0.54.

Using the 31-gene expression profile, this same criteria to evaluate what the genomic classification would be for the remaining 39 of 105 patients which were not included in the initial analysis (partial, minor, progressive disease and patients who were discontinued or crossed over) were applied. This was done using both the OKR and BKR best cytogenetic response scores (see Table 8). Results indicated that the majority of the partial (OKR=2) and minor (OKR=3) responders were classified as responders by genomic profiling (r<0.54), although the number of observations for both these categories closely matched the number expected (see Table 8). Interestingly, both of the patients that were classified as progressive disease (OKR=7) were also classified as responders, although the 2 such observations matched with the expected number of 2 cases (see Table 8). Results of Fisher's exact tests were significant in all cases (p<0.001) and also held up in follow-up analysis performed using the 18-month clinical data (see Table 9). Further analysis performed using classifications of major response (0-35% Ph+ cells) vs. minor to no response (>35% Ph+ cells, plus progressive disease) also resulted in significant associations between genomic classification and actual response, using either OKR or BKR cytogenetic data, for both the 12-month and 18-month data sets (p<0.001) as demonstrated in Table 10.

As demonstrated in FIG. 8, there was no association between disease progression and classification of response determined by correlation with the 31-gene expression profile. Indeed, 9 of the 10 individuals that demonstrated disease progression had an expression profile that correlated to positive cytogenetic response (r<0.54). Many of these individuals that had disease progression had also responded favorably to STI571, with some individuals showing complete or partial cytogenetic response (see Table 11). The reason for disease progression was not always directly related to loss of cytogenetic response as indicated in Table 11.

Interestingly, many of the genes identified in this report as having different expression between responders and non-responders appear to be functionally related to the pathogenesis of CML via the BCR-ABL oncogene. It is believed that the BCR-ABL oncogene can lead to malignant transformation via three major mechanisms: altered cell adhesion; constitutively active mitogenic signaling; and inhibition of apoptosis. See Faderl et al. (1999), supra; Deininger et al. (2000), supra; and Kabaraowski and Witte (2000), supra, for reviews.

One gene of particular interest is the CBL gene, which is a prominent target of the BCR-ABL oncogene and is capable of mediating a number of distinct signal transduction pathways. See Bhat et al., J. Biol. Chem., Vol. 272, No. 26, pp. 16170-16175 (1997). In this study, it was found that patients that responded to STI571 treatment (CCyR group) displayed increased expression of the CBL gene compared to the NoCyR group. Interestingly, another isoform of the CBL gene (CBLB) was identified as being differentially expressed in CCyR and NoCyR in STI-treated CML patients in a recent Japanese study. See Kaneta et al., (2002), supra. In this study, they performed a similar type of analysis to ours in a population of 22 Japanese CML patients enrolled in a Phase II clinical trial. Using 18 of these patients as a “learning” set, they identified a list of 79 genes that could predict which individuals achieved MCyR, and further confirmed their findings with the remaining 4 “test” patients. Comparing their list of 79 genes to the list of 55 differentially-expressed genes (Tables 12A and 12B), there was one gene in common between the two lists: CBL and its homolog CBLB. Interestingly, while isoform (CBL) was upregulated in the responder group, the Japanese group's isoform (CBLB) was expressed to a lower degree in responders compared to non-responders. This corresponds well with studies that have shown that CBL and CBLB are differentially-expressed in BCR-ABL transformed cells, and that the two isoforms act through different signal transduction pathways. See Sattler et al., Oncogene, Vol. 21, No. 9, pp. 1423-1433 (2002).

In conclusion, this analysis has identified a group of genes that are differentially-expressed between patients that achieved CCyR following treatment with STI571 and those that had minimal or NoCyR.

TABLE 12A
Summary of mean expression values for genes 1-31

| | | | | | | Pearson | ANOVA | Fold | Mean | | StdErr | |
|---|---|---|---|---|---|---|---|---|---|---|---|---|
| Order | Probe Set | Accession | Gene | Locus | Description | r | p-value | Inc/Dec | CCyR | NoCyR | CCyR | NoCyR |
| 1 | 37542_at | D86961 | LHFPL2 | 5q13.3 | lipoma HMGIC fusion partner-like 2 | −0.467 | 0.0016 | ↓ 3.5 | 27.9 | 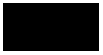 | 6.0 | 23.7 |
| 2 | 36741_at | D63482 | GIT2 | 12q24.1 | G protein-coupled receptor kinase-Interactor 2 | −0.444 | 0.0099 | ↓ 2.7 | 55.3 | 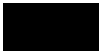 | 8.5 | 33.0 |
| 3 | 39692_at | AL080209 | DKFZp586F2423 | 7q34 | hypothetical protein DKFZp586F2423 | −0.412 | 0.0032 | ↓ 2.0 | 140.7 | 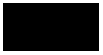 | 14.7 | 47.6 |
| 4 | 37050_r_at | AI130910 | TOMM34 | 20 | translocase of outer mitochondrial membrane 34 | −0.412 | 0.0085 | ↓ 3.0 | 33.0 | 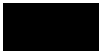 | 6.7 | 25.7 |
| 5 | 34376_at | AB019517 | PKIG | 20q12-q13.1 | protein kinase (cAMP-dependent, catalytic) inhibitor gamma | −0.376 | 0.0043 | ↓ 2.4 | 185.2 | 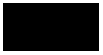 | 24.5 | 125.0 |
| 6 | 41614_at | AB014608 | KIAA0708 | 6p12.3 | KIAA0708 protein | −0.370 | 0.0105 | ↓ 2.1 | 182.5 | 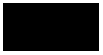 | 24.1 | 74.9 |
| 7 | 33346_r_at | M61764 | TUBG1 | 17q21 | tubulin, gamma 1 | −0.363 | 0.0108 | ↓ 1.8 | 189.2 | 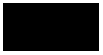 | 18.9 | 53.4 |
| 8 | 1269_at | M61906 | PIK3R1 | 5q12-q13 | phosphoinositide-3-kinase, regulatory subunit, polypeptide 1 (p85 alpha) | 0.357 | 0.0016 | ↑ 4.1 | 87.7 | 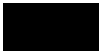 | 10.5 | 7.4 |
| 9 | 32849_at | D80000 | SMC1L1 | Xp11.22-p11.21 | SMC1 structural maintenance of chromosomes 1- like 1 (yeast) | −0.337 | 0.0132 | ↓ 2.4 | 43.5 | 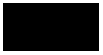 | 7.6 | 30.9 |
| 10 | 38986_at | Z49835 | GRP58 | 15q15 | glucose regulated protein. 58kD | −0.335 | 0.0021 | ↓ 2.1 | 100.1 | 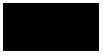 | 16.4 | 37.5 |
| 11 | 32847_at | U48959 | MYLK | 3q21 | myosin, light polypeptide kinase | −0.334 | 0.0268 | ↓ 3.5 | 22.1 | 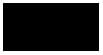 | 6.7 | 29.0 |
| 12 | 33949_at | AF011406 | CRHR2 | 7p15.1 | corticotropin releasing hormone receptor 2 | 0.333 | 0.0095 | ↑ 7.1 | 104.0 | 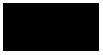 | 15.5 | 5.1 |
| 13 | 351_f_at | D28423 | SFRS3 | 11 | splicing factor, arginine/serine-rich 3, 5′UTR (sequence from the 5′cap to the start codon) | −0.327 | 0.0112 | ↓ 3.2 | 62.2 | 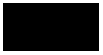 | 16.7 | 74.1 |
| 14 | 31919_at | AF002986 | H963 | 3q26.1 | platelet activating receptor homolog | −0.322 | 0.0324 | ↓ 2.1 | 108.3 | 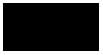 | 14.7 | 64.5 |
| 15 | 38226_at | W27152 | FLJ10569 | 8p21.2 | hypothetical protein FLJ10569 | −0.318 | 0.0403 | ↓ 2.0 | 133.3 | 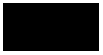 | 17.9 | 77.3 |
| 16 | 37535_at | M27691 | CREB1 | 2q32.3-q34 | cAMP responsive element binding protein 1 | −0.311 | 0.0154 | ↓ 1.8 | 101.4 | 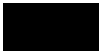 | 12.7 | 31.4 |
| 17 | 32158_at | U53174 | RAD9 | 11q13.1-q13.2 | RAD9 homolog (S. pombe) | −0.306 | 0.0423 | ↓ 24 | 60.8 | 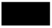 | 11.6 | 50.2 |
| 18 | 35180_at | AL050205 | LOC113251 | 12q13.12-q13.13 | c-Mpl binding protein | −0.301 | 0.0064 | ↓ 1.9 | 109.4 | 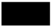 | 16.2 | 40.1 |
| 19 | 39341_at | AJ001902 | TRIP6 | 7q22 | thyroid hormone receptor interactor 6 | 0.300 | 0.0063 | ↑ 2.6 | 420.3 | 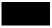 | 48.6 | 64.2 |
| 20 | 1457_at | M64174 | JAK1 | 1p32.3-p31.3 | Janus kinase 1 (a protein tyrosine kinase) | −0.291 | 0.0118 | ↓ 2.1 | 75.7 | 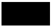 | 14.9 | 39.2 |
| 21 | 38467_at | U96721 | HPS | 10q23.1-q23.3 | Hermansky-Pudlak syndrome | 0.287 | 0.0109 | ↑ 1.8 | 539.6 | 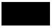 | 47.9 | 44.8 |
| 22 | 34077_at | X95876 | CXCR3 | Xq13 | Cytokine (C-X-C motif) receptor 3 | 0.285 | 0.0069 | ↑ 3.1 | 281.0 | 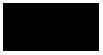 | 38.1 | 44.4 |
| 23 | 1558_g_at | U24152 | PAK1 | 11q13-q14 | p21/Cdc42/Rac1-activated kinase 1 (STE20 homolog, yeast) | 0.278 | 0.0113 | ↑ 2.7 | 115.7 | 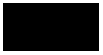 | 14.7 | 19.8 |
| 24 | 39867_at | S75463 | TUFM | 16p11.2 | Tu translation elongation factor, mitochondrial | −0.271 | 0.0032 | ↓ 1.7 | 279.3 | 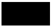 | 39.0 | 73.6 |
| 25 | 623_s_at | M28213 | RAB2 | 8q12.1 | RAB2, member RAS oncogene family | −0.268 | 0.0114 | ↓ 2.6 | 40.0 | 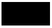 | 9.7 | 41.0 |
| 26 | 32518_at | AF019767 | ZNF259 | 11q23.3 | zinc finger protein 259 | 0.266 | 0.0119 | ↑ 2.0 | 398.7 | 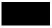 | 41.8 | 50.2 |
| 27 | 40793_s_at | U34846 | AQP4 | 18q11.2-q12.1 | aquaporin 4 | 0.264 | 0.0099 | ↑ 4.1 | 72.7 | 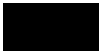 | 12.2 | 9.2 |
| 28 | 36303_f_at | U35376 | ZNF85 | 19p13.1-p12 | zinc finger protein 85 (HPF4, HTF1) | 0.263 | 0.0351 | ↑ 3.4 | 160.4 | 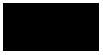 | 25.3 | 13.9 |
| 29 | 1305_s_at | D12620 | CYP4F3 | 19p13.2 | cytochrome P450, subfamily IVF, polypeptide 3 (leukotriene B4 omega hydroxylase) | 0.260 | 0.0224 | ↑ 1.8 | 293.7 | 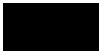 | 26.4 | 53.6 |
| 30 | 34416_at | X57110 | CBL | 11q23.3 | Cas-Br-M (murine) ecotropic retroviral transforming sequence | 0.260 | 0.0422 | ↑ 2.0 | 357.3 | 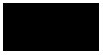 | 40.7 | 36.7 |
| 31 | 36120_at | X63657 | FVT1 | 18q21.3 | follicular lymphoma variant translocation 1 | −0.258 | 0.0151 | ↓ 2.1 | 53.2 | 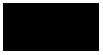 | 11.5 | 31.4 |

TABLE 12B
Summary of mean expression values for genes 32-55

| | | | | | | Pear- | | Fold | | | | |
|---|---|---|---|---|---|---|---|---|---|---|---|---|
| Or- | Probe | | | | | son | ANOVA | Inc/ | Mean | | StdErr | |
| der | Set | Accession | Gene | Locus | Description | r | p-value | Dec | CCyR | NoCyR | CCyR | NoCyR |
| 32 | 38298_at | U25138 | KCNMB1 | 5q34 | potassium large | 0.257 | 0.0299 | ↑ | 1022.4 | 478.4 | 120.6 | 152.5 |
| | | | | | conductance calcium- | | | 2.1 | | | | |
| | | | | | activated channel, | | | | | | | |
| | | | | | subfamily M, beta | | | | | | | |
| | | | | | member 1 | | | | | | | |
| 33 | 32641_at | AB023196 | AS3 | 13q12.3 | androgen-induced | 0.255 | 0.0175 | ↑ | 94.8 | 31.2 | 14.6 | 10.9 |
| | | | | | prostate proliferative | | | 3.0 | | | | |
| | | | | | shutoff associated | | | | | | | |
| | | | | | protein | | | | | | | |
| 34 | 41167_at | M64929 | PPP2R2A | 8p21.1 | protein phosphatase | 0.255 | 0.0495 | ↑ | 148.0 | 52.4 | 21.5 | 26.2 |
| | | | | | 2 (formerly 2A), | | | 2.8 | | | | |
| | | | | | regulatory subunit | | | | | | | |
| | | | | | B (PR 52), alpha | | | | | | | |
| | | | | | isoform | | | | | | | |
| 35 | 33597_at | U09411 | ZNF132 | 19q13.4 | zinc finger protein | −0.254 | 0.0391 | ↓ | 84.7 | 157.9 | 14.6 | 37.7 |
| | | | | | 132 (clone pHZ-12) | | | 1.9 | | | | |
| 36 | 41534_at | AB006755 | PCDH7 | 4p15 | BH-protocadherin | 0.253 | 0.0383 | ↑ | 256.9 | 132.1 | 28.8 | 24.0 |
| | | | | | (brain-heart) | | | 1.9 | | | | |
| 37 | 41137_at | AB007972 | PPP1R12B | 1q32.1 | protein phosphatase | 0.251 | 0.0159 | ↑ | 335.6 | 176.3 | 37.1 | 30.1 |
| | | | | | 1, regulatory | | | 1.9 | | | | |
| | | | | | (inhibitor) | | | | | | | |
| | | | | | subunit 12B | | | | | | | |
| 38 | 1856_at | X75042 | REL | 2p13- | v-rel reticuloendo- | −0.247 | 0.0279 | ↓ | 69.0 | 142.8 | 13.5 | 49.0 |
| | | | | p12 | theliosis viral | | | 2.1 | | | | |
| | | | | | oncogene homolog | | | | | | | |
| | | | | | (avian) | | | | | | | |
| 39 | 39980_at | AB000449 | VRK1 | 14q32 | vaccinia related | −0.247 | 0.0181 | ↓ | 58.8 | 121.6 | 13.7 | 27.6 |
| | | | | | kinase 1 | | | 2.1 | | | | |
| 40 | 38419_at | D83780 | KIAA0196 | 8p22 | KIAA0196 gene product | 0.240 | 0.0103 | ↑ | 62.5 | 17.8 | 11.0 | 7.1 |
| | | | | | | | | 3.5 | | | | |
| 41 | 31742_at | AF064090 | TNFSF14 | 19p13.3 | tumor necrosis factor | −0.237 | 0.0057 | ↓ | 63.5 | 134.6 | 14.6 | 44.3 |
| | | | | | | | | 2.1 | | | | |
| | | | | | (ligand) superfamily, | | | | | | | |
| | | | | | member 14 | | | | | | | |
| 42 | 36078_at | AL080120 | DKFZP584O0 | 11q13.4 | DKFZP564O0423 protein | 0.235 | 0.0327 | ↑ | 277.2 | 153.0 | 30.5 | 36.8 |
| | | | 423 | | | | | 1.8 | | | | |
| 43 | 617_at | M24902 | ACPP | 3q21- | acid phosphatase, | −0.228 | 0.0389 | ↓ | 74.7 | 140.4 | 14.2 | 41.5 |
| | | | | q23 | prostate | | | 1.9 | | | | |
| 44 | 322_at | D88532 | PIK3R3 | 1p33 | phosphoinositide-3- | 0.228 | 0.0413 | ↑ | 150.3 | 33.3 | 30.7 | 12.6 |
| | | | | | kinase, regulatory | | | 4.5 | | | | |
| | | | | | subunit, polypeptide | | | | | | | |
| | | | | | 3 (p55, gamma) | | | | | | | |
| 45 | 36839_at | U77949 | CDC6 | 17q21.3 | CDC6 cell division | 0.218 | 0.0464 | ↑ | 333.5 | 194.5 | 35.4 | 62.7 |
| | | | | | cycle 6 homolog | | | 1.7 | | | | |
| | | | | | (S. cerevisiae) | | | | | | | |
| 46 | 36052_at | U43959 | ADD2 | 2p14- | adducin 2 (beta) | 0.216 | 0.0090 | ↑ | 147.9 | 41.6 | 28.9 | 25.2 |
| | | | | p13 | | | | 3.6 | | | | |
| 47 | 37640_at | M31642 | HPRT1 | Xq26.1 | hypoxanthine phospho- | 0.216 | 0.0214 | ↑ | 123.2 | 66.8 | 14.5 | 25.1 |
| | | | | | ribosyltransferase | | | 1.8 | | | | |
| | | | | | 1 (Lesch-Nyhan | | | | | | | |
| | | | | | syndrome) | | | | | | | |
| 48 | 1328_at | U69108 | TRAF5 | 1q32 | TNF receptor- | 0.208 | 0.0318 | ↑ | 59.4 | 21.2 | 10.8 | 10.5 |
| | | | | | associated factor 5 | | | 2.8 | | | | |
| 49 | 35777_at | AB000468 | RNF4 | 4p16.3 | ring finger protein 4 | −0.200 | 0.0292 | ↓ | 89.8 | 158.7 | 19.3 | 31.8 |
| | | | | | | | | 1.8 | | | | |
| 50 | 36886_{—} | L41268 | KIR2DL3 | 19q13.4 | killer cell immuno- | 0.196 | 0.0340 | ↑ | 230.8 | 128.9 | 30.2 | 37.4 |
| | f_at | | | | globulin-like | | | 1.8 | | | | |
| | | | | | receptor, two | | | | | | | |
| | | | | | domains, long cyto- | | | | | | | |
| | | | | | plasmic tail, 3 | | | | | | | |
| 51 | 39019_at | D14696 | LAPTM4A | 2p24.3 | lysosomal-associated | −0.194 | 0.0271 | ↓ | 135.3 | 230.8 | 27.5 | 48.3 |
| | | | | | protein transmembrane | | | 1.7 | | | | |
| | | | | | 4 alpha | | | | | | | |
| 52 | 38581_at | U40038 | GNAQ | 9q21 | guanine nucleotide | −0.187 | 0.0263 | ↓ | 59.1 | 109.0 | 14.4 | 30.4 |
| | | | | | binding protein | | | 1.8 | | | | |
| | | | | | (G protein), q | | | | | | | |
| | | | | | polypeptide | | | | | | | |
| 53 | 38875_{—} | AB011147 | KIAA0575 | 2p25.1 | KIAA0575 gene product | 0.182 | 0.0359 | ↑ | 76.6 | 19.7 | 18.9 | 8.4 |
| | r_at | | | | | | | 3.9 | | | | |
| 54 | 40589_at | U40572 | SNTB2 | 16q22- | syntrophin, beta 2 | 0.170 | 0.0228 | ↑ | 139.1 | 70.4 | 23.1 | 34.5 |
| | | | | q23 | | | | 2.0 | | | | |
| | | | | | (dystrophin-associated | | | | | | | |
| | | | | | protein A1, 59 kD, | | | | | | | |
| | | | | | basic component 2) | | | | | | | |
| 55 | 31425_{—} | AC004853 | OR2F1 | 7q35 | olfactory receptor, | 0.148 | 0.0210 | ↑ | 133.6 | 77.3 | 19.5 | 53.3 |
| | g_at | | | | family 2, subfamily | | | 1.7 | | | | |
| | | | | | F, member 1 | | | | | | | |

Data from 66 samples used to determine genomic profile of cytogenetic response (53 CCyR, 13 NoCyR). Order = gene order by absolute correlation with response status, with 1 = highest correlation. Shading indicates the mean NoCyR expression profile used to classify response (Genes 1-31 only).

Additional RNA Isolation and cDNA Synthesis Techniques

Those of skill in the art will know of many techniques for the isolation of RNA. For example the following references describe several such procedures and the are hereby incorporated by reference for all purposes: Lockhart D. J., and Winzeler, E. A. (2000) Genomics, gene expression and DNA arrays. Nature 405,827-836, Lockhart, D. J., et al. (1996) Nature Biotech. 14, 1675-1680, Schmitt, M. E., et al (1990) A rapid and simple method for preparation of RNA from S. cerevisiae. Nucl. Acids Res. 18, 3091-3092, and Farrell, R. (1998) RNA Methodologies, Academic Press.

One such technique is disclosed below in some detail. In some embodiments the following technique can be used to extract RNA from patient samples, including blood, and synthesize cDNA; as follows;

RNA Isolation

1. Remove blood from −80° C. and thaw in 37° C. water bath by shaking and inversion.
2. Add 6 mL of whole blood to 22.5 mL of Tri Reagent BD (in a properly labeled 50 mL high-speed centrifuge tube).
3. Add 0.6 mL of 5 N acetic acid.
4. Close the tube and mix by inversion followed by brief vortexing (samples can be stored at −70° C. for several months at this point).
5. Allow samples to stand for 10 minutes at room temperature.
4. Add 3 mL 1-bromo-3-chloropropane to each sample.
5. Cover the sample tightly, invert vigorously for 15 seconds.
6. Allow samples to stand for 5 minutes at room temperature.
7. Centrifuge resulting mixture at 9,000×g for 18 minutes at 4° C. as centrifuge tubes are prone to leaks and sample is especially precious. The mixture will separate into three phases: red organic phase (protein), an interphase (DNA), and a colorless upper aqueous phase (RNA).
   RNA Precipitation
1. Transfer aqueous phase to new, properly labeled 50 ml high speed centrifuge tube.
2. Add 16.8 mL isopropanol, and mix briefly by inversion.
3. Allow sample to stand at room temperature for 5-10 minutes and precipitate overnight at 20° C.
4. Remove sample from −20° C. and let stand at room temperature for 5 minutes.
5. Centrifuge at 9,000×g for 10 minutes at 4° C. The RNA precipitate will form a pellet on the side and bottom of the tube.
   RNA Wash
1. Pipet off as much of the supernatant as possible without disturbing the pellets.
2. Add 15 mL of 75% ethanol and vortex the sample.
3. Centrifuge at 7,500×g for 8 minutes at 4° C.
4. Pipet off half of the supernatant (leaving about 8-12 mL), making sure not to disturb the pellet.
5. Re-suspend pellet in the 75% ethanol by pulsing up and down with 2 mL pipet (be sure to scrape down the sides of the tube with the pipet tip to re-suspend all RNA).
6. Aliquot each sample into four 1.5 mL microcentrifuge tubes.
7. Spin down tubes for 10 minutes at full speed.
8. Pipet off (and save) supernatant.
9. Add another aliquot to each tube.
10. Repeat process until the entire sample is used. Recombine the pellets.
11. Do one final 75% ethanol wash.
12. Spin down for 5 minutes at full speed.
13. Pipet off the ethanol.
   RNA Solubilization
1. Air dry the pellet for 5-10 minutes.
2. Re-suspend in 100 μL of warm DEPC water. If sample does not re-suspend readily, heat for 5 minutes in 65° C. and pipet up and down vigorously.
3. Briefly spin down.
4. Quantitate by absorption at 260/280 nM and run out 300 ng on a 1% agarose gel.
5. Proceed to purifying samples using Rneasy columns.
   Purification of Total RNA
1. Total RNA is purified using RNeasy Mini Spin Columns (Qiagen). Load no more than 100 μg of total RNA on the column. The sample volume is adjusted to 100 μL with RNase-free water.
2. Add 350 μL of Buffer RLT to the sample and mix thoroughly.
3. Add 250 μL ethanol (100%) to the lysate and mix well by pipetting. Do not centrifuge.
4. Apply sample (700 μL) to an RNeasy mini spin column sitting in a collection tube. Centrifuge for 15 seconds at ≧8,000×g (≧10,000 rpm).
5. Reapply sample flow-through (700 μL) to the RNeasy mini spin column. Centrifuge for 15 seconds at ≧8,000×g (≧10,000 rpm). Discard flow-through and collection tube.
6. Transfer the RNeasy column to a new 2 mL collection tube (supplied). Add 500 μL Buffer RPE and centrifuge for 15 seconds at ≧8,000×g (≧10,000 rpm) to wash. Discard flow-through.
7. Pipette 500 μL Buffer RPE and centrifuge for 2 minutes at maximum speed to dry the membrane. Discard flow-through and spin the sample for 1 minute to ensure dryness.

Discard collection tube.
8. Transfer the RNeasy column into a new 1.5 mL collection tube (supplied) and pipette 30 μL of warm RNase-free water (65° C.) directly onto the RNeasy membrane. Let the column sit for 1 minute. Centrifuge for 1 minute at ≧8,000×g (≧10,000 rpm) for 2 minutes to elute RNA.
9. Re-apply the flow-through back onto the column let stand for 1 minute. Re-centrifuge at ≧8,000×g (≧10,000 rpm) for 2 minutes.
10. The concentration and quantity of the sample is taken using a DU 650 Spectrophotometer (Beckman Coulter). Samples should be at a concentration of 0.5 μg/μL or greater. A minimum of 5 μg of total RNA is necessary to perform cDNA synthesis.
11. Three hundred ng of the total RNA is run on a 1% agarose gel to check the quality. SYBER Green II stain (Molecular Probes) is used to stain the gel.
12. Quantitate sample and run out on gel again.
13. Proceed to cDNA synthesis or store at −80° C. until further processing.
   cDNA Synthesis

Full length total RNA is used to synthesize double-stranded cDNA using the Superscript Choice System (available from Life Technologies).

First strand cDNA synthesis

| Total RNA (μg) | Superscript II RT (μL), 200 U/μL |
|---|---|
| 5.0-8.0 | 1.0 |
| 8.1-16.0 | 2.0 |
| 16.1-24.0 | 3.0 |
| 24.1-32.0 | 4.0 |
| 32.1-40.0 | 5.0 |

X+Y+Z=12
1. Add total RNA (X)
   Appropriate amount of DEPC water (Y)
   1 μL 100 pmol/μL T7-(T)24 primer
   (T)24)(GENOSYS™)
2. Mix and heat at 70° C. for 10 minutes.

3. Add the following to the RNA/primer mix:

| Reagent | Volume (μL) |
|---|---|
| 5 × 1^{st} strand buffer | 4.0 |
| 0.1 mM DTT | 2.0 |
| 10 mM dNTPs | 1.0 |

4. Heat at 42° C. for 2 minutes
5. Add the appropriate amounts of SSII RT (400 U total) (Life Technologies) (Z)
6. Mix and heat at 42° C. for 1 hour.
   Second Strand cDNA Synthesis
1. Put all reagents and 1^{st} strand tubes on ice

2. Add to 1^{st} strand tubes

| Reagent | Volume (μL) |
|---|---|
| DEPC H₂O | 91.0 |
| 5 × 2^{nd} strand buffer | 30.0 |
| E. coli DNA POL I (40 U) | 4.0 |
| 10 mM dNTPs | 3.0 |
| E. coli DNA Ligase (10 U) | 1.0 |
| E. coli RNase H (2 U) | 1.0 |

3. Incubate at 16° C. for 2 hours
4. Add 2 μL (10 U) T4 DNA Polymerase and incubate at 16° C. for 5 minutes
5. Add 10 μL 0.5 M EDTA to stop the reaction.
6. Store at −20° C. until further processing.
   PLG-Phenol/Chloroform Extraction of cDNA
1. Pellet the Phase Lock Gel (PLG) in a microcentrifuge at 12,000×g for one minute.
2. Add an equal volume (162 μL) of (25:24:1) phenol:chloroform:isoamyl alcohol (saturated with 10 mM Tris-HCL pH 8.0/1 mM EDTA-Sigma) to the cDNA sample. Vortex sample.
3. Transfer the entire mixture into the labeled PLG tube.
4. Microcentrifuge at maximum speed (12,000×g or greater) for 2 minutes.
5. Transfer the aqueous upper phase to a new, labeled 1.5 mL tube. Continue on with the EtOH precipitation.
   EtOH Precipitation of cDNA
1. Add 2 μL of 5 mg/mL glycogen to your sample as a carrier.
2. Add 0.5 volume of 7.5 M NH₄Ac to your sample (81 μL).
3. Add 2.5 volumes of cold absolute ethanol (stored at −200) and vortex the sample (405 μL).
4. Immediately centrifuge at full speed for 20 minutes at room temperature.
5. Remove the supernatant by pipetting it out carefully. Wash the pellet in 0.5 mL 80% cold ethanol (stored at −200). Centrifuge at maximum speed for 5 minutes.
6. Remove the 80% ethanol by pipetting it out very carefully, as the pellet may be loose. Repeat the 80% ethanol wash one additional time.
7. Remove the 80% ethanol by pipetting it out very carefully, the pellet may be loose. Air-dry the pellet. Check for dryness before proceeding.
8. Re-suspend pellet in 12 μL of warm DEPC water.
   cRNA Synthesis

The cDNA is transcribed in vitro using Enzo BIO-ARRAY” High Yield RNA transcript Labeling Kit (ENZO) to form biotin-labeled cRNA. The following table is used to calculate the amount of cDNA to use in the IVT reaction based on the original amount of purified RNA used.

cDNA in IVT (total RNA)

| Total RNA (μg) | Volume of cDNA to use in IVT (μL)* |
|---|---|
| 5-8 | 10 |
| 8.1-16 | 5 |
| 16.1-24 | 3.3 |
| 24.1-32 | 2.5 |
| 32.1-40 | 2 |

*Assuming 12 μL re-suspension volume for cDNA.

1. Add reaction components to clean, labeled microcentrifuge tubes, which are kept at room temperature while additions are made. Make additions in the order indicated.

| Reagent | Volume (μL) |
|---|---|
| CDNA | See tables above for amount |
| DEPC H₂O | Variable depending on |
| | amount of cDNA |
| 10 × HY reaction buffer | 4.0 |
| 10 × biotin-labeled | 4.0 |
| ribonucleotides | |
| 10 × DTT | 4.0 |
| 10 × RNase inhibitor mix | 4.0 |
| 20 × T7 RNA polymerase enzyme | 2.0 |
| Total volume | 40.0 |

2. Carefully mix the reagents and collect the mixture in the bottom of the tube by brief microcentrifugation.
3. Immediately place the tube in a 37° C. water bath. Incubate for 5 hours. Gently mix the contents every 3045 minutes during incubation.
4. Stop the reaction by adding 1.5 μL of 0.5 M EDTA and 1.5 μL of 10% SDS.
5. Purify the cRNA reaction with RNeasy Mini Spin Columns.
6. The concentration of the sample is taken using a DU 650 spectrophotometer (Beckman Coulter).
7. Three hundred ng of the total RNA is run on a 1% agarose gel to check quality. SYBER Green II stain (Molecular Probes) is used to stain the gel.
   Fragmenting cRNA and Preparation of Hybridization Cocktail

Affymetrix recommends that the RNA used in the fragmentation procedure be sufficiently concentrated to maintain a small volume during the procedure. This will minimize the amount of magnesium in the final hybridization cocktail. The cRNA must be at a minimum concentration of 0.6 μg/μL when you start fragmenting.

Fragmenting cRNA for target preparation. Add 2 μL of 5×fragmentation buffer (2) for every 8 μL of RNA plus H₂O. The final concentration of RNA in the fragmentation mix can range from 0.5-2 μg/μL. The following table shows an example of a fragmentation mix for a 20 μg cRNA sample at a final concentration of 0.5 μg/μL.

| Component | Volume (μL) |
|---|---|
| 20 μg cRNA | 1-32 |
| 5 × fragmentation buffer | 8 |
| RNase-free H₂O | to 40 |
| Final concentration | 0.5 μg/μL |

1. Incubate at 94° C. for 35 minutes. Place on ice following fragmentation. Store undiluted fragmented sample RNA at −20° C. until ready to hybridize.

2. Mix the following for each target.

| | | |
|---|---|---|
| | Standard | Final |
| Component | array | concentration |

| Fragmented cRNA | 12-15 | μg | 0.05 | μg/μL |
|---|---|---|---|---|
| Control oligo B2 (5 nM) | 3 | μL | 50 | pM |
| 100 × control cRNA cocktail* | 3 | μL | 1.5, 5, 25 | |
| | | | and 100 pM | |
| | | | for Bio B, | |
| | | | C, D and Cre, | |
| | | | respectively | |
| Herring sperm DNA (10 mg/MI) | 3 | μL | 0.1 | mg/mL |
| Acetylated BSA (50 mg/mL) | 3 | μL | 0.5 | mg/mL |

| 2 × MES hybridization buffer | 150 | μL | 1× |
|---|---|---|---|
| H₂O | To a final | | |
| | volume of | | |
| | 300 | μL | |

*It is imperative that frozen stocks of 100 × control cRNA cocktail be heated to 65° C. for 5 minutes to completely re-suspend the cRNA.

Target Cleanup and Hybridization
1. Equilibrate probe array to room temperature immediately before use.
2. Heat the hybridization cocktail to 99° C. for 5 minutes in a heat block.
3. Meanwhile, wet the array by filling it with 200 μL of 1×MES hybridization buffer (100 mM MES, 1 M [Na⁺], 20 mM EDTA, 0.01% Tween 20). Incubate the probe array at 45° C. for 10 minutes with rotation.
4. After incubation of hybridization cocktail at 99° C., transfer the hybridization cocktail to the 45° C. hybridization oven for 5 minutes.
5. Spin hybridization cocktail at maximum speed in a microcentrifuge for 5 minutes.
6. Remove the buffer solution from the probe array cartridge and fill with 200 μL of hybridization cocktail avoiding any unsoluble material at the bottom of the tube.
7. Place probe array in rotisserie box in 45° C. oven. Hybridize for 16 hours overnight.
   Measurement Methods

The experimental methods of this invention depend on measurements of cellular constituents. The cellular constituents measured can be from any aspect of the biological state of a cell. They can be from the transcriptional state, in which RNA abundances are measured, the translation state, in which protein abundances are measured, the activity state, in which protein activities are measured. The cellular characteristics can also be from mixed aspects, for example, in which the activities of one or more proteins are measured along with the RNA abundances (gene expressions) of other cellular constituents. This section describes exemplary methods for measuring the cellular constituents in drug or pathway responses. This invention is adaptable to other methods of such measurement.

Preferably, in this invention the transcriptional state of the other cellular constituents is measured. The transcriptional state can be measured by techniques of hybridization to arrays of nucleic acid or nucleic acid mimic probes, described in the next subsection, or by other gene expression technologies, described in the subsequent subsection. However measured, the result is data including values representing mRNA abundance and/or ratios, which usually reflect DNA expression ratios (in the absence of differences in RNA degradation rates).

In various alternative embodiments of the present invention, aspects of the biological state other than the transcriptional state, such as the translational state, the activity state or mixed aspects can be measured.

Cell-free assays can also be used to identify compounds which are capable of interacting with a protein encoded by one of the disclosed genes in Table 6 or Tables 12A or 12B or protein binding partner, to alter the activity of the protein or its binding partner. Cell-free assays can also be used to identify compounds, which modulate the interaction between the encoded protein and its binding partner such as a target peptide.

In one embodiment, cell-free assays for identifying such compounds comprise a reaction mixture containing a protein encoded by one of the disclosed genes and a test compound or a library of test compounds in the presence or absence of the binding partner, e.g., a biologically inactive target peptide or a small molecule. Accordingly, one example of a cell-free method for identifying agents useful in the treatment of breast cancer is provided which comprises contacting a protein or functional fragment thereof or the protein binding partner with a test compound or library of test compounds and detecting the formation of complexes. For detection purposes, the protein can be labeled with a specific marker and the test compound or library of test compounds labeled with a different marker. Interaction of a test compound with the protein or fragment thereof or the protein binding partner can then be detected by measuring the level of the two labels after incubation and washing steps. The presence of the two labels is indicative of an interaction.

Interaction between molecules can also be assessed by using real-time BIA (Biomolecular Interaction Analysis, Pharmacia Biosensor (AB) which detects surface plasmon resonance, an optical phenomenon. Detection depends on changes in the mass concentration of mass macromolecules at the biospecific interface and does not require labeling of the molecules. In one useful embodiment, a library of test compounds can be immobilized on a sensor surface, e.g., a wall of a micro-flow cell. A solution containing the protein, functional fragment thereof, or the protein binding partner is then continuously circulated over the sensor surface. An alteration in the resonance angle, as indicated on a signal recording, indicates the occurrence of an interaction. This technique is described in more detail in “BIAtechnology Handbook” by Pharmacia.

Another embodiment of a cell-free assay comprises: a) combining a protein encoded by the at least one gene, the protein binding partner and a test compound to form a reaction mixture; and b) detecting interaction of the protein and the protein binding partner in the presence and absence of the test compounds. A considerable change (potentiation or inhibition) in the interaction of the protein and binding partner in the presence of the test compound compared to the interaction in the absence of the test compound indicates a potential agonist (mimetic or potentiator) or antagonist (inhibitor) of the proteins' activity for the test compound. The components of the assay can be combined simultaneously or the protein can be contacted with the test compound for a period of time, followed by the addition of the binding partner to the reaction mixture. The efficacy of the compound can be assessed by using various concentrations of the compound to generate dose response curves. A control assay can also be performed by quantitating the formation of the complex between the protein and its binding partner in the absence of the test compound.

Formation of a complex between the protein and its binding partner can be detected by using detectably labeled proteins such as radiolabeled, fluorescently-labeled or enzymatically-labeled protein or its binding partner, by immunoassay or by chromatographic detection.

In preferred embodiments, the protein or its binding partner can be immobilized to facilitate separation of complexes from uncomplexed forms of the protein and its binding partner and automation of the assay. Complexation of the protein to its binding partner can be achieved in any type of vessel, e.g., microtitre plates, micro-centrifuge tubes and test tubes. In particularly preferred embodiment, the protein can be fused to another protein, e.g., glutathione-S-transferase to form a fusion protein which can be absorbed onto a matrix, e.g., glutathione sepharose beads (Sigma Chemical, St. Louis, Mo.) which are then combined with the labeled protein partner, e.g., labeled with ³⁵S, and test compound and incubated under conditions sufficient to formaton of complexes. Subsequently, the beads are washed to remove unbound label and the matrix is immobilized and the radiolabel is determined.

Another method for immobilizing proteins on matrices involves utilizing biotin and streptavidin. For example, the protein can be biotinylated using biotin NHS(N-hydroxy-succinimide) using well-known techniques and immobilized in the well of steptavidin-coated plates.

Cell-free assays can also be used to identify agents which are capable of interacting with a protein encoded by the at least one gene and modulate the activity of the protein encoded by the gene. In one embodiment, the protein is incubated with a test compound and the catalytic activity of the protein is determined. In another embodiment, the binding affinity of the protein to a target molecule can be determined by methods known in the art.

As used herein the term “antisense” refers to nucleotide sequences that are complementary to a portion of an RNA expression product of at least one of the disclosed genes. “Complementary” nucleotide sequences refer to nucleotide sequences that are capable of base-pairing according to the standard Watson-Crick complementary rules. That is, purines will base-pair with pyrimidine to form combinations of guanine:cytosine and adenine:thymine in the case of DNA, or adenine:uracil in the case of RNA. Other less common bases, e.g., inosine, 5-methylcytosine, 6-methyladenine, hypoxanthine and others may be included in the hybridizing sequences and will not interfere with pairing.

In all embodiments, measurements of the cellular constituents should be made in a manner that is relatively independent of when the measurements are made.

Transcriptional State Measurement

Preferably, measurement of the transcriptional state is made by hybridization of nucleic acids to oligonucleotide arrays, which are described in this subsection. Certain other methods of transcriptional state measurement are described later in this subsection.

Transcript Arrays Generally

In a preferred embodiment the present invention makes use of “oligonucleotide arrays” (also called herein “microarrays”). Microarrays can be employed for analyzing the transcriptional state in a cell, and especially for measuring the transcriptional states of cancer cells.

In one embodiment, transcript arrays are produced by hybridizing detectably-labeled polynucleotides representing the mRNA transcripts present in a cell (e.g., fluorescently-labeled cDNA synthesized from total cell mRNA or labeled cRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics: The arrays are reproducible, allowing multiple copies of a given array to be produced and easily compared with each other. Preferably the microarrays are small, usually smaller than 5 cm², and they are made from materials that are stable under binding (e.g., nucleic acid hybridization) conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. In a specific embodiment, positionally addressable arrays containing affixed nucleic acids of known sequence at each location are used.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA or cRNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

Preparation of Microarrays

Microarrays are known in the art and consist of a surface to which probes that correspond in sequence to gene products (e.g., cDNAs, mRNAs, cRNAs, polypeptides and fragments thereof), can be specifically hybridized or bound at a known position. In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA or cRNA can specifically hybridize. The nucleic acid or analogue of the binding site can be, e.g., a synthetic oligomer, a full-length cDNA, a less-than full-length cDNA, or a gene fragment.

Although in a preferred embodiment the microarray contains binding sites for products of all or almost all genes in the target organism's genome, such comprehensiveness is not necessarily required. The microarray may have binding sites for only a fraction of the genes in the target organism. However, in general, the microarray will have binding sites corresponding to at least about 50% of the genes in the genome, often at least about 75%, more often at least about 85%, even more often more than about 90% and most often at least about 99%. Preferably, the microarray has binding sites for genes relevant to testing and confirming a biological network model of interest. A “gene” is identified as an open reading frame (ORF) of preferably at least 50, 75 or 99 amino acids from which a mRNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6275 ORFs longer than 99 amino acids. Analysis of these ORFs Indicates that there are 5885 ORFs that are likely to specify protein products (Goffeau et al., “Life with 6000 Genes”, Science, Vol. 274, pp. 546-567 (1996), which is incorporated by reference in its entirety for all purposes). In contrast, the human genome is estimated to contain approximately 25,000-35,000 genes.

Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences or the sequences may be synthesized de novo on the surface of the chip, for example by use of photolithography techniques, e.g., Affymetrix uses such a different technology to synthesize their oligos directly on the chip). PCR primers are chosen, based on the known sequence of the genes or cDNA, that result in amplification of unique fragments (i.e., fragments that do not share more than 10 bases of contiguous identical sequence with any other fragment on the microarray). Computer programs are useful in the design of primers with the required specificity and optimal amplification properties (see, e.g., Oligo pI version 5.0, National Biosciences). In the case of binding sites corresponding to very long genes, it will sometimes be desirable to amplify segments near the 3′ end of the gene so that when oligo-dT primed cDNA probes are hybridized to the microarray; less-than-full length probes will bind efficiently. Typically each gene fragment on the microarray will be between about 20 bp and about 2000 bp, more typically between about 100 bp and about 1000 bp, and usually between about 300 bp and about 800 bp in length. PCR methods are well known and are described, for example, in Innis et al. eds., PCR Protocols: A Guide to Methods and Applications, Academic Press Inc. San Diego, Calif. (1990), which is incorporated by reference in its entirety for all purposes. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (see Froehler et al., Nucleic Acid Res, Vol. 14, pp. 5399-5407 (1986); McBride et al., Tetra. Lett., Vol. 24, pp. 245-248 (1983)). Synthetic sequences are between about 15 and about 500 bases in length, more typically between about 20 and about 50 bases. In some embodiments, synthetic nucleic adds include non-natural bases, e.g., inosine. As noted above, nucleic acid analogues may be used as binding sites for hybridization. An example of a suitable nucleic acid analogue is peptide nucleic acid (see, e.g., Egholm et al., “PNA Hybridizes to Complementary Oligonucleotides Obeying the Watson-Crick Hydrogen-Bonding Rules”, Nature, Vol. 365, pp. 566-568 (1993); see also U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., “Differential Gene Expression in the Murine Thymus Assayed by Quantitative Hybridization of Arrayed cDNA Clones”, Genomics, Vol. 29, pp. 207-209 (1995)). In yet another embodiment, the polynucleotide of the binding sites is RNA.

Attaching Nucleic Acids to the Solid Surface

The nucleic acid or analogue are attached to a solid support, which may be made from glass, plastic (e.g., polypropylene, nylon), polyacrylamide, nitrocellulose or other materials. A preferred method for attaching the nucleic acids to a surface is by printing on glass plates, as is described generally by Schena et al., “Quantitative Monitoring of Gene Expression Patterns with a Complementary DNA Microarray”, Science, Vol. 270, pp. 467-470 (1995). This method is especially useful for preparing microarrays of cDNA (see, also, DeRisi et al., “Use of a cDNA Microarray to Analyze Gene Expression Patterns in Human Cancer”, Nature Gen., Vol. 14, pp. 457-460 (1996); Shalon et al., “A DNA Microarray System for Analyzing Complex DNA Samples Using Two-Color Fluorescent Probe Hybridization”, Genome Res., Vol. 6, pp. 639-645 (1996); and Schena et al., “Parallel Human Genome Analysis; Microarray-Based Expression of 1000 Genes”, Proc. Natl. Acad. Sci. USA, Vol. 93, pp. 10539-11286 (1995)). Each of the aforementioned articles is incorporated by reference in its entirety for all purposes.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., “Light-Directed Spatially Addressable Parallel Chemical Synthesis”, Science, Vol. 251, pp. 767-773 (1991); Pease et al., “Light-Directed Oligonucleotide Arrays for Rapid DNA Sequence Analysis”, Proc. Natl. Acad. Sci. USA, Vol. 91, pp. 5022-5026 (1994); Lockhart et al., “Expression Monitoring by Hybridization to High-Density Oligonucleotide Arrays”, Nature Biotech., Vol. 14, p. 1675 (1996); U.S. Pat. Nos. 5,578,832; 5,556,752; and 5,510,270, each of which is incorporated by reference in its entirety for all purposes) or other methods for rapid synthesis and deposition of defined oligonucleotides (see Blanchard et al., “High-Density Oligonucleotide Arrays”, Biosensors & Bioelectronics, Vol. 11, pp. 687-690 (1996)). When these methods are used, oligonucleotides (e.g., 25 mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (see Maskos et al., Nuc. Acids Res., Vol. 20, pp. 1679-1684 (1992)), may also be used. In principal, any type of array, for example, dot blots on a nylon hybridization membrane (see Sambrook et al., “Molecular Cloning—A Laboratory Manual”, 2^{nd} Ed., Vols. 1-3, Cold Spring Harbor Laboratory, Cold Spring Harbor, N.Y. (1989), which is incorporated in its entirety for all purposes), could be used, although, as will be recognized by those of skill in the art, very small arrays will be preferred because hybridization volumes will be smaller.

Generating Labeled Probes

Methods for preparing total and poly(A)+ RNA are well-known and are described generally in Sambrook et al., supra. In one embodiment, RNA is extracted from cells of the various types of interest in this invention using guanidinium thiocyanate lysis followed by CsCl centrifugation (see Chirgwin et al., Biochemistry, Vol. 18, pp. 5294-5299 (1979)). Poly(A)+ RNA is selected by selection with oligo-dT cellulose (see Sambrook et al., supra). Cells of interest include wild-type cells, drug-exposed wild-type cells, cells with modified/perturbed cellular constituent(s), and drug-exposed cells with modified/perturbed cellular constituent(s).

Labeled cDNA is prepared from mRNA or alternatively directly from RNA by oligo dT-primed or random-primed reverse transcription, both of which are well-known in the art (see, e.g., Klug et al., Methods Enzymol., Vol. 152, pp. 316-325 (1987)). Reverse transcription may be carried out in the presence of a dNTP conjugated to a detectable label, most preferably a fluorescently-labeled dNTP. Alternatively, isolated mRNA can be converted to labeled antisense RNA synthesized by in vitro transcription of double-stranded cDNA in the presence of labeled dNTPs (see Lockhart et al., “Expression Monitoring by Hybridization to High-Density Oligonucleotide Arrays”, Nature Biotech., Vol. 14, p. 1675 (1996), which is incorporated by reference in its entirety for all purposes). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, Fluor X (Amersham) and others (see, e.g., Kricka, “Nonisotopic DNA Probe Techniques”, Academic Press, San Diego, Calif. (1992)). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., “High Density cDNA Filter Analysis: A Novel Approach for Large-Scale, Quantitative Analysis of Gene Expression”, Gene, Vol. 156, p. 207 (1995); Pietu et al., “Novel Gene Transcripts Preferentially Expressed in Human Muscles Revealed by Quantitative Hybridization of a High Density cDNA Array”, Genome Res., Vol. 6, p. 492 (1996)). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., ™II, LTI Inc.) at 42° C. for 60 minutes.

Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., supra).

Optimal hybridization conditions will depend on the length (e.g., oligomer vs. polynucleotide >200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-Interscience, NY (1987) which is incorporated in its entirety for all purposes. When the cDNA microarrays of Schena et al. are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1×SSC plus 0.2% SDS) (see Shena et al., Proc. Natl. Acad. Sci. USA, Vol. 93, p. 10614 (1996)). Useful hybridization conditions are also provided in, e.g., Tijessen, “Hybridization with Nucleic Acid Probes”, Elsevier Science Publishers B.V. and Kricka (1993); “Nonisotopic DNA Probe Techniques”, Academic Press, San Diego, Calif. (1992).

Signal Detection and Data Analysis

When fluorescently-labeled probes are used, the fluorescence emissions at each site of a transcript array can be, preferably, detected by scanning confocal laser microscopy. In one embodiment, a separate scan, using the appropriate excitation line, Is carried out for each of the two fluorophores used. Alternatively, a laser can be used that allows specimen illumination at wavelengths specific to the fluorophores used and emissions from the fluorophore can be analyzed. In a preferred embodiment, the arrays are scanned with a laser fluorescent scanner with a computer controlled X-Y stage and a microscope objective. Sequential excitation of the fluorophore is achieved with a multi-line, mixed gas laser and the emitted light is split by wavelength and detected with a photomultiplier tube. Fluorescence laser scanning devices are described in Schena et al., Genome Res., Vol. 6, pp. 639-645 (1996) and in other references cited herein. Alternatively, the fiber-optic bundle described by Ferguson et al., Nature Biotech., Vol. 14, pp. 1681-1684 (1996), may be used to monitor mRNA abundance levels at a large number of sites simultaneously.

Signals are recorded and, in a preferred embodiment, analyzed by computer, e.g., using a 12-bit analog to digital board. In one embodiment the scanned image is despeckled using a graphics program (e.g., Hijaak Graphics Suite) and then analyzed using an image gridding program that creates a spreadsheet of the average hybridization at each wavelength at each site.

The Agilent Technologies GENEARRAY™ scanner is a bench-top, 488 nM argon-ion laser-based analysis instrument. The laser can be focused to a spot size of less than 4 microns. This precision allows for the scanning of probe arrays with probe cells as small as 20 microns. The laser beam focuses onto the probe array, exciting the fluorescent-labeled nucleotides. It then and then scans using the selected filter for the dye used in the assay. Scanning in the orthogonal coordinate is achieved by moving the probe array. The laser radiation is absorbed by the dye molecules incorporated into the hybridized sample and causes them to emit fluorescence radiation. This fluorescent light is collimated by a lens and passes through a filter for wavelength selection. The light is then focused by a second lens onto an aperture for depth discrimination and then detected by a highly sensitive photo multiplier tube (PMT). The output current of the PMT is converted into a voltage read by an analog to digital converter (ADC) and the processed data is passed back to the computer as the fluorescent intensity level of the sample point, or picture element (pixel) currently being scanned. The computer displays the data as an image, as the scan progresses. In addition, the fluorescent intensity level of all samples, representing the expression profile of the sample, is recorded in computer readable format.

If necessary, an experimentally determined correction for “cross talk” (or overlap) between the channels for the two fluors may be made. For any particular hybridization site on the transcript array, a ratio of the emission of the two fluorophores may be calculated. The ratio is independent of the absolute expression level of the cognate gene, but may be useful for genes whose expression is significantly modulated by drug administration, gene deletion, or any other tested event.

Preferably, in addition to identifying a perturbation as positive or negative, it is advantageous to determine the magnitude of the perturbation. This can be carried out by methods that will be readily apparent to those of skill in the art.

Other Methods of Transcriptional State Measurement

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent 0 534858 A1, filed Sep. 24, 1992, by Zabeau et al.), or methods selecting restriction fragments with sites closest to a defined mRNA end (see, e.g., Prashar et al., Proc. Natl. Acad. Sci. USA, Vol. 93, pp. 659-663 (1996)). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see, e.g., Velculescu, Science, Vol. 270, pp. 484-487 (1995)) pathway pattern.

Measurement of Other Aspects

In various embodiments of the present invention, aspects of the biological state other than the transcriptional state, such as the translational state, the activity state or mixed aspects can be measured in order to obtain drug and pathway responses. Details of these embodiments are described in this section.

Translational State Measurements

Expression of the protein encoded by the gene(s) can be detected by a probe which is detectably-labeled, or which can be subsequently-labeled. Generally, the probe is an antibody that recognizes the expressed protein.

As used herein, the term “antibody” includes, but is not limited to, polyclonal antibodies, monoclonal antibodies, humanized or chimeric antibodies and biologically functional antibody fragments sufficient for binding of the antibody fragment to the protein.

For the production of antibodies to a protein encoded by one of the disclosed genes, various host animals may be immunized by injection with the polypeptide, or a portion thereof. Such host animals may include, but are not limited to, rabbits, mice, and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol and potentially useful human adjuvants such as BCG (bacille Camette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler et al., Nature, Vol. 256, pp. 495-497 (1975); and U.S. Pat. No. 4,376,110. The human B-cell hybridoma technique of Kosbor et al., Immunol. Today, Vol. 4, p. 72 (1983); Cole et al., Proc. Natl. Acad. Sci. USA, Vol. 80, pp. 2026-2030 (1983); and the EBV-hybridoma technique, Cole et al., Monoclonal Antibodies and Cancer Ther., Alan R. Liss, Inc., pp. 77-96 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies”, Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985), by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived form a murine mAb and a human immunoglobulin constant region.

Alternatively, techniques described for the production of single chain antibodies, U.S. Pat. No. 4,946,778; Bird, Science, Vol. 242, pp. 423426 (1988); Huston et al., Proc. Natl. Acad. Sci. USA, Vol. 85, pp. 5879-5883 (1988); and Ward et al., Nature, Vol. 334, pp. 544-546 (1989), can be adapted to produce differentially expressed gene single-chain antibodies. Single-chain antibodies are formed by linking the heavy and light chain fragments of the Fv region via an amino acid bridge, resulting in a single-chain polypeptide.

More preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments, which recognize specific epitopes, may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed, Huse et al., Science, Vol. 246, pp. 1275-1281 (1989), to allow rapid and easy identification of monoclonal Fab fragments with the desired specificity.

The extent to which the known proteins are expressed in the sample is then determined by immunoassay methods that utilize the antibodies described above. Such immunoassay methods include, but are not limited to, dot blotting, western blotting, competitive and noncompetitive protein-binding assays, enzyme-linked immunosorbant assays (ELISA), immunohistochemistry, fluorescence activated cell sorting (FACS), and others commonly used and widely described in scientific and patent literature, and many employed commercially.

Particularly preferred, for ease of detection, is the sandwich ELISA, of which a number of variations exist, all of which are intended to be encompassed by the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody must be an antibody that is specific for the protein expressed by the gene of interest.

The most commonly used reporter molecules in this type of assay are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate Is then added to the tertiary complex. The substrate reacts with the enzyme linked to the second antibody, giving a qualitative visual signal, which may be further quantitated, usually spectrophotometrically, to give an evaluation of the amount of protein which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

Measurement of the translational state may also be performed according to several additional methods. For example, whole genome monitoring of protein (i.e., the “proteome”, Goffeau et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to testing or confirming a biological network model of interest. Methods for making monoclonal antibodies are well-known (see, e.g., Harlow et al., “Antibodies: A Laboratory Manual”, Cold Spring Harbor, N.Y. (1988), which is incorporated in its entirety for all purposes). In a one preferred embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension (see, e.g., Hames et al., “Gel Electrophoresis of Proteins: A Practical Approach”, IRL Press, NY (1990); Shevchenko et al., Proc. Nat'l Acad. Sci. USA, Vol. 93, pp. 1440-1445 (1996); Sagliocco et al., Yeast, Vol. 12, pp. 1519-1533 (1996); Lander, Science, Vol. 274, pp. 536-539 (1996)). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

Embodiments Based on Other Aspects of the Biological State

Although monitoring cellular constituents other than mRNA abundances currently presents certain technical difficulties not encountered in monitoring mRNAs, it will be apparent to those of skill in the art that the use of methods of this invention that the activities of proteins relevant to the characterization of cell function can be measured, embodiments of this invention can be based on such measurements. Activity measurements can be performed by any functional, biochemical, or physical means appropriate to the particular activity being characterized. Where the activity involves a chemical transformation, the cellular protein can be contacted with the natural substrates, and the rate of transformation measured. Where the activity involves association in multimeric units, for example association of an activated DNA binding complex with DNA, the amount of associated protein or secondary consequences of the association, such as amounts of mRNA transcribed, can be measured. Also, where only a functional activity is known, for example, as in cell cycle control, performance of the function can be observed. However known and measured, the changes in protein activities form the response data analyzed by the foregoing methods of this invention.

In alternative and non-limiting embodiments, response data may be formed of mixed aspects of the biological state of a cell. Response data can be constructed from, e.g., changes in certain mRNA abundances, changes in certain protein abundances, and changes in certain protein activities.

Computer Implementations

In a preferred embodiment, the computation steps of the previous methods are implemented on a computer system or on one or more networked computer systems in order to provide a powerful and convenient facility for forming and testing models of biological systems. The computer system may be a single hardware platform comprising internal components and being linked to external components. The internal components of this computer system include processor element interconnected with a main memory. For example computer system can be an Intel Pentium based processor of 200 Mhz or greater clock rate and with 32 MB or more of main memory.

The external components include mass data storage. This mass storage can be one or more hard disks (which are typically packaged together with the processor and memory). Typically, such hard disks provide for at least 1 GB of storage. Other external components include user interface device, which can be a monitor and keyboards, together with pointing device, which can be a “mouse”, or other graphic input devices. Typically, the computer system is also linked to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on mass storage. Alternatively, the software components may be stored on removable media such as floppy disks or CD-ROM (not illustrated). The software component represents the operating system, which is responsible for managing the computer system and its network interconnections. This operating system can be, e.g., of the Microsoft Windows family, such as Windows 95, Windows 98 or Windows NT, or a Unix operating system, such as Sun Solaris. Software includes common languages and functions conveniently present on this system to assist programs implementing the methods specific to this invention. Languages that can be used to program the analytic methods of this invention include C, C++, or, less preferably, JAVA. Most preferably, the methods of this invention are programmed in mathematical software packages, which allow symbolic entry of equations and high-level specification of processing, including algorithms to be used, and thereby freeing a user of the need to procedurally program individual equations or algorithms. Such packages include, e.g., MATLAB™ from Mathworks (Natick, Mass.), MATHEMATICA™ from Wolfram Research (Champaign, Ill.) and MATHCAD™ from Mathsoft (Cambridge, Mass.).

In preferred embodiments, the analytic software component actually comprises separate software components that interact with each other. Analytic software represents a database containing all data necessary for the operation of the system. Such data will generally include, but is not necessarily limited to, results of prior experiments, genome data, experimental procedures and cost, and other information, which will be apparent to those skilled in the art. Analytic software includes a data reduction and computation component comprising one or more programs which execute the analytic methods of the invention. Analytic software also includes a user interface (UI) which provides a user of the computer system with control and input of test network models, and, optionally, experimental data. The user interface may comprise a drag-and-drop interface for specifying hypotheses to the system. The user interface may also comprise means for loading experimental data from the mass storage component (e.g., the hard drive), from removable media (e.g., floppy disks or CD-ROM), or from a different computer system communicating with the instant system over a network (e.g., a local area network, or a wide area communication network such as the internet).

Alternative computer systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

Identification and Characterization of SNPs

Many different techniques can be used to identify and characterize SNPs, including single-strand conformation polymorphism analysis, heteroduplex analysis by denaturing high-performance liquid chromatography (DHPLC), direct DNA sequencing and computational methods, see Shi M M, Clin Chem 2001, 47: 164-172. Thanks to the wealth of sequence information in public databases, computational tools can be used to identify SNPs in silico by aligning independently submitted sequences for a given gene (either cDNA or genomic sequences). Comparison of SNPs obtained experimentally and by in silico methods showed that 55% of candidate SNPs found by SNPFinder (http://lpgws.nci.nih.gov:821perl/snp/snp_cgi.pl) have also been discovered experimentally, see, Cox et al. Hum Mutal 2001, 17: 141-150. However, these in silico methods could only find 27% of true SNPs.

The most common SNP typing methods currently include hybridization, primer extension and cleavage methods. Each of these methods must be connected to an appropriate detection system. Detection technologies include fluorescent polarization, (see Chan X et al. Genome Res 1999, 9: 492-499), luminometric detection of pyrophosphate release (pyrosequencing), (see Ahmadiian A et al. Anal Biochem 2000, 280: 103-10), fluorescence resonance energy transfer (FRET)-based cleavage assays, DHPLC, and mass spectrometry, (see Shi M M, Clin Chem 2001, 47: 164-172 and U.S. Pat. No. 6,300,076 B1). Other methods of detecting and characterising SNPs are those disclosed in U.S. Pat. Nos. 6,297,018 B1 and 6,300,063 B1. The disclosures of the above references are incorporated herein by reference in their entirety.

In a particularly preferred embodiment the detection of the polymorphism can be accomplished by means of so called INVADER™ technology (available from Third Wave Technologies Inc. Madison, Wis.). In this assay, a specific upstream “invader” oligonucleotide and a partially overlapping downstream probe together form a specific structure when bound to complementary DNA template. This structure is recognized and cut at a specific site by the Cleavase enzyme, and this results in the release of the 5′ flap of the probe oligonucleotide. This fragment then serves as the “invader” oligonucleotide with respect to synthetic secondary targets and secondary fluorescently labeled signal probes contained in the reaction mixture. This results in specific cleavage of the secondary signal probes by the Cleavase enzyme. Fluoresence signal is generated when this secondary probe, labeled with dye molecules capable of fluorescence resonance energy transfer, is cleaved. Cleavases have stringent requirements relative to the structure formed by the overlapping DNA sequences or flaps and can, therefore, be used to specifically detect single base pair mismatches immediately upstream of the cleavage site on the downstream DNA strand. See Ryan D et al. Molecular Diagnosis Vol. 4 No 2 1999: 135-144 and Lyamichev V et al. Nature Biotechnology Vol 17 1999: 292-296, see also U.S. Pat. Nos. 5,846,717 and 6,001,567 (the disclosures of which are incorporated herein by reference in their entirety).

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

Genotyping oligonucleotides of the invention may also be immobilized on or synthesized on a solid surface such as a microchip, bead, or glass slide (see, e.g., WO 98/20020 and WO 98/20019). Such immobilized genotyping oligonucleotides may be used in a variety of polymorphism detection assays, including but not limited to probe hybridization and polymerase extension assays. Immobilized genotyping oligonucleotides of the invention may comprise an ordered array of oligonucleotides designed to rapidly screen a DNA sample for polymorphisms in multiple genes at the same time.

An allele-specific oligonucleotide primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. Allele-specific oligonucleotide primers hybridizing to either the coding or noncoding strand are contemplated by the invention. An ASO primer for detecting gene polymorphisms on the putative gene DKFZP434C131 in the 15q22.33 region, the CYP1A1gene, or the IL-1 beta can be developed using techniques known to those of skill in the art.

Other genotyping oligonucleotides of the invention hybridize to a target region located one to several nucleotides downstream of one of the novel polymorphic sites identified herein. Such oligonucleotides are useful in polymerase-mediated primer extension methods for detecting one of the novel polymorphisms described herein and therefore such genotyping oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

In another embodiment, the invention provides a kit comprising at least two genotyping oligonucleotides packaged in separate containers. The kit may also contain other components such as hybridization buffer (where the oligonucleotides are to be used as a probe) packaged in a separate container. Alternatively, where the oligonucleotides are to be used to amplify a target region, the kit may contain, packaged in separate containers, a polymerase and a reaction buffer optimized for primer extension mediated by the polymerase, such as PCR.

The above described oligonucleotide compositions and kits are useful in methods for genotyping and/or haplotyping the putative gene DKFZP434C131 in the 15q22.33 region, the CYP1A1 gene, or the IL-1 beta gene in an individual. As used herein, the terms “genotype” and “haplotype” mean the genotype or haplotype containing the nucleotide pair or nucleotide, respectively, that is present at one or more of the novel polymorphic sites described herein and may optionally also include the nucleotide pair or nucleotide present at one or more additional polymorphic sites in the putative gene DKFZP434C131 in the 15q22.33 region, the CYP1A1gene, or the IL-1 beta gene. The additional polymorphic sites may be currently known polymorphic sites or sites that are subsequently discovered.

One embodiment of the genotyping method involves isolating from the individual a nucleic acid mixture comprising the two copies of the genes of interest, i.e., the rs2290573 polymorphism on the putative gene DKFZP434C131 in the 15q22.33 region, the CYP1A1 gene at the polymorphic site at position 6819 in sequence X02612; and the IL-1 beta gene, at the polymorphic site at position 1423 of sequence X04500 or a fragment thereof, that are present in the individual, and determining the identity of the nucleotide pair at one or more of the polymorphic sites in the two copies to assign a genotype to the individual.

As will be readily understood by the skilled artisan, the two “copies” of a gene in an individual may be the same allele or may be different alleles. In a particularly preferred embodiment, the genotyping method comprises determining the identity of the nucleotide pair at each polymorphic site.

Typically, the nucleic acid mixture is isolated from a biological sample taken from the individual, such as a blood sample or tissue sample. Suitable tissue samples include whole blood, semen, saliva, tears, urine, fecal material, sweat, buccal smears, skin and hair. The nucleic acid mixture may be comprised of genomic DNA, mRNA, or cDNA and, in the latter two cases, the biological sample must be obtained from an organ in which the putative gene DKFZP434C131 in the 15q22.33 region, the CYP1A1 gene, or the IL-1 beta gene is expressed. Furthermore it will be understood by the skilled artisan that mRNA or cDNA preparations would not be used to detect polymorphisms located in introns or in 5′ and 3′ nontranscribed regions. If a gene fragment is isolated, it must contain the polymorphic site(s) to be genotyped.

One embodiment of the haplotyping method comprises isolating from the individual a nucleic acid molecule containing only one of the two copies of the gene, or a fragment thereof, that is present in the individual and determining in that copy the identity of the nucleotide at one or more of the polymorphic sites in that copy to assign a haplotype to the individual. The nucleic acid may be isolated using any method capable of separating the two copies of the gene or fragment, including but not limited to, one of the methods described above for preparing isogenes, with targeted in vivo cloning being the preferred approach. As will be readily appreciated by those skilled in the art, any individual clone will only provide haplotype information on one of the two gene copies present in an individual. If haplotype information is desired for the individuals other copy, additional clones will need to be examined. Typically, at least five clones should be examined to have more than a 90% probability of haplotyping both copies of the gene in an individual. In a particularly preferred embodiment, the nucleotide at each of polymorphic site is identified.

In a preferred embodiment, a haplotype pair is determined for an individual by identifying the phased sequence of nucleotides at one or more of the polymorphic sites in each copy of the gene that is present in the individual. In a particularly preferred embodiment, the haplotyping method comprises identifying the phased sequence of nucleotides at each polymorphic site in each copy of the gene. When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both the genotyping and haplotyping methods, the identity of a nucleotide (or nucleotide pair) at a polymorphic site(s) may be determined by amplifying a target region(s) containing the polymorphic site(s) directly from one or both copies of the gene of interest, ie, the rs2290573 polymorphism on the putative gene DKFZP434C131 in the 15q22.33 region, the CYP1A1gene at the polymorphic site at position 6819 in sequence X02612; and the IL-1 beta gene, at the polymorphic site at position 1423 of sequence X04500, or fragment thereof, and the sequence of the amplified region(s) determined by conventional methods.

It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for all individual homozygous at that site, or both guanine and cytosine, if the individual is heterozygous at that site. Alternatively, the site may be negatively determined to be not guanine (and thus cytosine/cytosine) or not cytosine (and thus guanine/guanine).

In addition, the identity of the allele(s) present at any of the novel polymorphic sites described herein may be indirectly determined by genotyping a polymorphic site not disclosed herein that is in linkage disequilibrium with the polymorphic site that is of interest. Two sites are said to be in linkage disequilibrium if the presence of a particular variant at one site enhances the predictability of another variant at the second site (See, Stevens, J C 1999, Mol Diag 4: 309-317). Polymorphic sites in linkage disequilibrium with the presently disclosed polymorphic sites may be located in regions of the gene or in other genomic regions not examined herein. Genotyping of a polymorphic site in linkage disequilibrium with the novel polymorphic sites described herein may be performed by, but is not limited to, any of the above-mentioned methods for detecting the identity of the allele at a polymorphic site.

The target region(s) may be amplified using any oligonucleotide-directed amplification method, including but not limited to polymerase chain reaction (PCR) (U.S. Pat. No. 4,965,188), ligase chain reaction (LCR) (See, Barany et al., Proc Natl Acad Sci USA 88: 189-193, 1991; and WO 90/01069), and oligonucleotide ligation assay (OLA) (Landegren et al., Science 241: 1077-1080, 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (See, U.S. Pat. No. 5,130,238; EP 329,822; U.S. Pat. No. 5,169,766, WO 89/06700) and isothermal methods (Walker et al., Proc Nat Acad Sci USA 89: 392-396, 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within 5° C. and more preferably within 2° C., of each other when hybridizing to each of the polymorphic sites being detected.

Hybridization of an allele-specific oligonucleotide to a target polynucleotide may be performed with both entities in solution or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype or haplotype for the gene or interest of an individual may also be determined by hybridization of a nucleic sample containing one or both copies of the gene to nucleic acid arrays and subarrays such as described in WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., Proc Natl Acad Sci USA 82: 7575, 1985; Meyers et al., Science 230: 1242, 1985) and proteins which recognize nucleotide mismatches, such as the E. coli mutS protein (Modrich P. Ann Rev Genet 25: 229-253, 1991). Alternatively, variant alleles can be identified by single strand conformation polymorphism (SSCP) analysis (Orita et al., Genomics 5: 874-879, 1989; Humphries et al., in Molecular Diagnosis of Genetic Diseases, R. Elles, ed., pp. 321-340, 1996) or denaturing gradient gel electrophoresis (DGGE) (Wartell et at., Nucl Acids Res 18: 2699-2706, 1990; Sheffield et al., Proc Natl Acad Sci USA 86: 232-236, 1989).

A polymerase-mediated primer extension method may also be used to identify the polymorphism(s). Several such methods have been described in the patent and scientific literature and include the “Genetic Bit Analysis” method (WO 92/15712) and the ligase/polymerase mediated genetic bit analysis (U.S. Pat. No. 5,679,524). Related methods are disclosed in WO 91/02087, WO 90/09455, WO 95/17676, U.S. Pat. Nos. 5,302,509 and 5,945,283. Extended primers containing a polymorphism may be detected by mass spectrometry as described in U.S. Pat. No. 5,605,798. Another primer extension method is allele-specific PCR (Ruafio et al., Nucl Acids Res 17: 8392, 1989; Ruafio et al., Nucl Acids Res 19, 6877-6882, 1991; WO 93/22456; Turki et al., I Clin Invest 95: 1635-1641, 1995). In addition, multiple polymorphic sites may be investigated by simultaneously amplifying multiple regions of the nucleic acid using sets of allele-specific primers as described in Wallace et al. (WO 89/10414).

In a preferred embodiment, the haplotype frequency data for each ethnogeographic group is examined to determine whether it is consistent with Hardy-Weinberg equilibrium. Hardy-Weinberg equilibrium (D. L. Hartl et al., Principles of Population Genomics, Sinauer Associates (Sunderland, Mass.), 3rd Ed., 1997) postulates that the frequency of finding the haplotype pair H₁/H₂ is equal to P_{H-W}(H₁/H₂)=2p(H₁)p(H₂) if H₁≠H₂ and P_{H-W}(H₁/H₂)=p (H₁)p(H₂) if H₁=H₂. A statistically significant difference between the observed and expected haplotype frequencies could be due to one or more factors including significant inbreeding in the population group, strong selective pressure on the gene, sampling bias, and/or errors in the genotyping process. If large deviations from Hardy-Weinberg equilibrium are observed in an ethnogeographic group, the number of individuals in that group can be increased to see if the deviation is due to a sampling bias. If a larger sample size does not reduce the difference between observed and expected haplotype pair frequencies, then one may wish to consider haplotyping the individual using a direct haplotyping method such as, for example, CLASPER System™ technology (U.S. Pat. No. 5,866,404), SMD, or allele-specific long-range PCR (Michalotos-Beloin et al., Nucl Acids Res 24: 4841-4843, 1996).

In one embodiment of this method for predicting a haplotype pair of the genes of interesy, i.e. the rs2290573 polymorphism on the putative gene DKFZP434C131 in the 15q22.33 region, the CYP1A1 gene at the polymorphic site at position 6819 in sequence X02612; and the IL-1 beta gene, at the polymorphic site at position 1423 of sequence X04500, the assigning step involves performing the following analysis. First, each of the possible haplotype pairs is compared to the haplotype pairs in the reference population. Generally, only one of the haplotype pairs in the reference population matches a possible haplotype pair and that pair is assigned to the individual. Occasionally, only one haplotype represented in the reference haplotype pairs is consistent with a possible haplotype pair for an Individual, and in such cases the individual is assigned a haplotype pair containing this known haplotype and a new haplotype derived by subtracting the known haplotype from the possible haplotype pair. In rare cases, either no haplotypes in the reference population are consistent with the possible haplotype pairs, or alternatively, multiple reference haplotype pairs are consistent with the possible haplotype pairs. In such cases, the individual is preferably haplotyped using a direct molecular haplotyping method such as, for example, CLASPER System™ technology (U.S. Pat. No. 5,866,404), SMD, or allele-specific long-range PCR (Michalotos-Beloin et al., Nucl Acids Res 24: 4841-4843, 1996).

The invention also provides a method for determining the frequency of a genotype or haplotype of interest in a population. The method comprises determining the genotype or the haplotype pair for the gene of interest that is present in each member of the population, wherein the genotype or haplotype comprises the nucleotide pair or nucleotide detected at one or more of the polymorphic sites in the gene of interest, including but not limited to; the rs2290573 polymorphism on the putative gene DKFZP434C131 in the 15q22.33 region, the CYP1A1gene at the polymorphic site at position 6819 in sequence X02612; and the IL-1beta gene, at the polymorphic site at position 1423 of sequence X04500, and calculating the frequency any particular genotype or haplotype is found in the population. The population may be a reference population, a family population, a same sex population, a population group, a trait population (e.g., a group of individuals exhibiting a trait of interest such as a medical condition or response to a therapeutic treatment).

In another aspect of the invention, frequency data for genotypes and/or haplotypes of interest found in a reference population are used in a method for identifying an association between a trait and a genotype or a haplotype of interest. The trait may be any detectable phenotype, including but not limited to susceptibility to a disease or response to a treatment The method involves obtaining data on the frequency of the genotype(s) or haplotype(s) of interest in a reference population as well as in a population exhibiting the trait. Frequency data for one or both of the reference and trait populations may be obtained by genotyping or haplotyping each individual in the populations using one of the methods described above. The haplotypes for the trait population may be determined directly or, alternatively, by the predictive genotype to haplotype approach described above.

In another embodiment, the frequency data for the reference and/or trait populations is obtained by accessing previously determined frequency data, which may be in written or electronic form. For example, the frequency data may be present in a database that is accessible by a computer. Once the frequency data is obtained, the frequencies of the genotype(s) or haplotype(s) of interest in the reference and trait populations are compared. In a preferred embodiment, the frequencies of all genotypes and/or haplotypes observed in the populations are compared. If a particular genotype or haplotype for the gene of interest is more frequent in the trait population than in the reference population at a statistically significant amount, then the trait is predicted to be associated with that genotype or haplotype.

In a preferred embodiment statistical analysis is performed by the use of standard ANOVA tests with a Bonferoni correction and/or a bootstrapping method that simulates the genotype phenotype correlation many times and calculates a significance value. When many polymorphisms are being analyzed a correction to factor may be performed to correct for a significant association that might be found by chance. For statistical methods for use in the methods of this invention, see: Statistical Methods in Biology, 3^{rd} edition, Bailey N T J, Cambridge Univ. Press (1997); Introduction to Computational Biology, Waterman M S, CRC Press (2000) and Bioinformatics, Baxevanis A D and Ouellette B F F editors (2001) John Wiley & Sons, Inc.

In a preferred embodiment of the method, the trait of interest is a clinical response exhibited by a patient to some therapeutic treatment, for example, response to a tyrosine kinase inhibitor drug or response to a therapeutic treatment for a medical condition.

In another embodiment of the invention, a detectable genotype or haplotype that is in linkage disequilibrium with the any of the genotypes or haplotypes of interest may be used as a surrogate marker. A genotype that is in linkage disequilibrium with a genotype of interest may be discovered by determining if a particular genotype or haplotype for the gene is more frequent in the population that also demonstrates the potential surrogate marker genotype than in the reference population at a statistically significant amount, then the marker genotype is predicted to be associated with that genotype or haplotype and then can be used as a surrogate marker in place of the genotype of interest, in prefered embodiments this would include, but not be limited to; the rs2290573 polymorphism on the putative gene DKFZP434C131 in the 15q22.33 region, the CYP1A1gene at the polymorphic site at position 6819 in sequence X02612; and the IL-1 beta gene, at the polymorphic site at position 1423 of sequence X04500.

As used herein, “medical condition” includes but is not limited to any condition or disease manifested as one or more physical and/or psychological symptoms for which treatment is desirable, and includes previously and newly identified diseases and other disorders.

As used herein, the term “clinical response” means any or all of the following: a quantitative measure of the response, no response, and adverse response (i.e., side effects).

In order to deduce a correlation between clinical response to a treatment and a genotype or haplotype of Interesy it is necessary to obtain data on the clinical responses exhibited by a population of individuals who received the treatment, hereinafter the “clinical population”. This clinical data may be obtained by analyzing the results of a clinical trial that has already been run and/or the clinical data may be obtained by designing and carrying out one or more new clinical trials.

As used herein, the term “clinical trial” means any research study designed to collect clinical data on responses to a particular treatment, and includes but is not limited to phase 1, phase II and phase III clinical trials. Standard methods are used to define the patent population and to enroll subjects.

It is preferred that the individuals included in the clinical population have been graded for the existence of the medical condition of interest. This is important in cases where the symptom(s) being presented by the patients can be caused by more than one underlying condition, and where treatment of the underlying conditions are not the same. An example of this would be where patients experience breathing difficulties that are due to either asthma or respiratory infections. If both sets were treated with an asthma medication, there would be a spurious group of apparent non-responders that did not actually have asthma. These people would affect the ability to detect any correlation between haplotype and treatment outcome. This grading of potential patients could employ a standard physical exam or one or more lab tests. Alternatively, grading of patients could use haplotyping for situations where there is a strong correlation between haplotype pair and disease susceptibility or severity.

The therapeutic treatment of interest is administered to each individual in the trial population and each individual's response to the treatment is measured using one or more predetermined criteria. It is contemplated that in many cases, the trial population will exhibit a range of responses and that the investigator will choose the number of responder groups (e.g., low, medium, high) made up by the various responses. In addition, the gene of interest for each individual in the trial population is genotyped and/or haplotyped, which may be done before or after administering the treatment.

After both the clinical and polymorphism data have been obtained, correlations between individual response and genotype or haplotype content are created, including but not limited to; the rs2290573 polymorphism on the putative gene DKFZP434C131 in the 15q22.33 region, the CYP1A1gene at the polymorphic site at position 6819 in sequence X02612; and the IL-1 beta gene, at the polymorphic site at position 1423 of sequence X04500.

Correlations may be produced in several ways. In one method, individuals are grouped by their genotype or haplotype (or haplotype pair) (also referred to as a polymorphism group), and then the averages and standard deviations of clinical responses exhibited by the members of each polymorphism group are calculated.

These results are then analyzed to determine if any observed variation in clinical response between polymorphism groups is statistically significant. Statistical analysis methods which may be used are described in L. D. Fisher and G. vanBelle, “Biostatistics: A Methodology for the Health Sciences”, Wiley-Interscience (New York) 1993. This analysis may also include a regression calculation of which polymorphic sites in the putative gene DKFZP434C131 in the 15q22.33 region, the CYP1A1 gene, or the IL-1 beta gene give the most significant contribution to the differences in phenotype. One regression model useful in the invention is described in the PCT Application entitled “Methods for Obtaining and Using Haplotype Data”, filed Jun. 26, 2000.

A second method for finding correlations between haplotype content and clinical responses uses predictive models based on error-minimizing optimization algorithms. One of many possible optimization algorithms is a genetic algorithm (R. Judson, “Genetic Algorithms and Their Uses in Chemistry” in Reviews in Computational Chemistry, Vol. 10, pp. 1-73, K. B. Lipkowitz and D. B. Boyd, eds. (VCH Publishers, New York, 1997). Simulated annealing (Press et al., “Numerical Recipes in C: The Art of Scientific Computing”, Cambridge University Press (Cambridge) 1992, Ch. 10), neural networks (E. Rich and K. Knight, “Artificial Intelligence”, 2nd Edition (McGraw-Hill, New York, 1991, Ch. 18), standard gradient descent methods (Press et al., supra Ch. 10), or other global or local optimization approaches (see discussion in Judson, supra) could also be used. Preferably, the correlation is found using a genetic algorithm approach as described in PCT Application entitled “Methods for Obtaining and Using Haplotype Data”, filed Jun. 26, 2000.

Correlations may also be analyzed using analysis of variation (ANOVA) techniques to determine how much of the variation in the clinical data is explained by different subsets of the polymorphic sites in the genes of interest. As described in PCT Application entitled “Methods for Obtaining and Using Haplotype Data”, filed Jun. 26, 2000, ANOVA Is used to test hypotheses about whether a response variable is caused by or correlated with one or more traits or variables that can be measured (Fisher and vanBelle, supra, Ch. 10).

From the analyses described above, a mathematical model may be readily constructed by the skilled artisan that predicts clinical response as a function of genotype or haplotype content. Preferably, the model is validated in one or more follow-up clinical trials designed to test the model.

The identification of an association between a clinical response and a genotype or haplotype (or haplotype pair) for the putative gene DKFZP434C131 in the 15q22.33 region, the CYP1A1gene, or the IL-1 beta gene may be the basis for designing a diagnostic method to determine those individuals who will or will not respond to the treatment, or alternatively, will respond at a lower level and thus may require more treatment, i.e., a greater dose of a drug.

The diagnostic method may take one of several forms: for example, a direct DNA test (i.e., genotyping or haplotyping one or more of the polymorphic sites in the rs2290573 polymorphism on the putative gene DKFZP434C131 in the 15q22.33 region, the CYP1A1 gene at the polymorphic site at position 6819 in sequence X02612; and the IL-1 beta gene, at the polymorphic site at position 1423 of sequence X04500), a serological test, or a physical exam measurement.

The only requirement is that there be a good correlation between the diagnostic test results and the underlying genotype or haplotype that is in turn correlated with the clinical response. In a preferred embodiment, this diagnostic method uses the predictive haplotyping method described above.

A computer may implement any or all analytical and mathematical operations involved in practicing the methods of the present invention. In addition, the computer may execute a program that generates views (or screens) displayed on a display device and with which the user can interact to view and analyze large amounts of information relating to the genes of interest and its genomic variation, including chromosome location, gene structure, and gene family, gene expression data, polymorphism data, genetic sequence data, and clinical data population data (e.g., data on ethnogeographic origin, clinical responses, genotypes, and haplotypes for one or more populations).

The genetic polymorphism data described herein may be stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

In other embodiments, the invention provides methods, compositions, and kits for haplotyping and/or genotyping the genes of interest in an individual, including; the rs2290573 polymorphism on the putative gene DKFZP434C131 in the 15q22.33 region, the CYP1A1gene at the polymorphic site at position 6819 in sequence X02612; and the IL-1beta gene, at the polymorphic site at position 1423 of sequence X04500.

The methods involve identifying the nucleotide or nucleotide pair present at the rs2290573 polymorphism on the putative gene DKFZP434C131 in the 15q22.33 region, in the CYP1A1gene at the polymorphic site at position 6819 in sequence X02612; and in the IL-1 beta gene, at the polymorphic site at position 1423 of sequence X04500. The compositions contain oligonucleotide probes and primers designed to specifically hybridize to one or more target regions containing, or that are adjacent to, a polymorphic site.

The methods and compositions for establishing the genotype or haplotype of an individual at the novel polymorphic sites described herein are useful for studying the effect of the polymorphisms in the etiology of diseases affected by the expression and function of the varioue gene expression products including, but not limited to proteins, studying the efficacy of drugs targeting these protiens, predicting individual susceptibility to diseases affected by the expression and function of the expression protein Including tyrosine kinases and predicting individual responsiveness to drugs targeting the identified targets.

In yet another embodiment, the invention provides a method for identifying an association between a genotype or haplotype and a trait. In preferred embodiments, the trait is susceptibility to a disease, severity of a disease, the staging of a disease or response to a drug. Such methods have applicability in developing diagnostic tests and therapeutic treatments for all pharmacogenetic applications where there is the potential for an association between a genotype and a treatment outcome including efficacy measurements, PK measurements and side effect measurements.

The present invention also provides a computer system for storing and displaying polymorphism data determined for the genes of interest. The computer system comprises a computer processing unit; a display; and a database containing the polymorphism data. The polymorphism data includes the polymorphisms, the genotypes and the haplotypes identified for the rs2290573 polymorphism on the putative gene DKFZP434C131 in the 15q22.33 region, the CYP1A1 gene at the polymorphic site at position 6819 in sequence X02612; and the IL-1 beta gene, at the polymorphic site at position 1423 of sequence X04500 gene in a reference population. In a preferred embodiment, the computer system is capable of producing a display showing various haplotypes organized according to their evolutionary relationships.

In another aspect, the invention provides SNP probes, which are useful in classifying people according to their types of genetic variation. The SNP probes according to the invention are oligonucleotides, which can discriminate between alleles of a SNP nucleic acid in conventional allelic discrimination assays.

As used herein, a “SNP nucleic acid” is a nucleic acid sequence, which comprises a nucleotide that is variable within an otherwise identical nucleotide sequence between individuals or groups of individuals, thus, existing as alleles. Such SNP nucleic acids are preferably from about 15 to about 500 nucleotides in length. The SNP nucleic acids may be part of a chromosome, or they may be an exact copy of a part of a chromosome, e.g., by amplification of such a part of a chromosome through PCR or through cloning. The SNP nucleic acids are referred to hereafter simply as “SNPs”. The SNP probes according to the invention are oligonucleotides that are complementary to a SNP nucleic acid.

As used herein, the term complementary means exactly complementary throughout the length of the oligonucleotide in the Watson and Crick sense of the word.

In certain preferred embodiments, the oligonucleotides according to this aspect of the invention are complementary to one allele of the SNP nucleic acid, but not to any other allele of the SNP nucleic acid. Oligonucleotides according to this embodiment of the invention can discriminate between alleles of the SNP nucleic acid in various ways. For example, under stringent hybridization conditions, an oligonucleotide of appropriate length will hybridize to one allele of the SNP nucleic acid, but not to any other allele of the SNP nucleic acid. The oligonucleotide may be labeled by a radiolabel or a fluorescent label. Alternatively, an oligonucleotide of appropriate length can be used as a primer for PCR, wherein the 3′ terminal nucleotide is complementary to one allele of the SNP nucleic acid, but not to any other allele. In this embodiment, the presence or absence of amplification by PCR determines the haplotype of the SNP nucleic acid.

Thus, in one embodiment, the invention provides an isolated polynucleotide comprising a nucleotide sequence that is a polymorphic variant of a reference sequence for the genes of interest or fragments thereof. The reference sequence comprises the standard or most common sequence and the polymorphic variant comprises at least one polymorphism, including but not limited to the rs2290573 polymorphism on the putative gene DKFZP434C131 in the 15q22.33 region, the CYP1A1gene at the polymorphic site at position 6819 in sequence X02612; and the IL-1 beta gene, at the polymorphic site at position 1423 of sequence X04500.

Genomic and cDNA fragments of the invention comprise at least one novel polymorphic site identified herein and have a length of at least 10 nucleotides and may range up to the full length of the gene. Preferably, a fragment according to the present invention is between 100 and 3000 nucleotides in length, and more preferably between 200 and 2000 nucleotides in length, and most preferably between 500 and 1000 nucleotides in length.

In describing the polymorphic sites identified herein reference is made to the sense strand of the gene for convenience. However, as recognized by the skilled artisan, nucleic acid molecules containing the genes of interest may be complementary double stranded molecules and thus reference to a particular site on the sense strand refers as well to the corresponding site on the complementary antisense strand. Thus, reference may be made to the same polymorphic site on either strand and an oligonucleotide may be designed to hybridize specifically to either strand at a target region containing the polymorphic site. Thus, the invention also includes single-stranded polynucleotides that are complementary to the sense strand of the various genomic variants described herein.

In a further aspect of the invention there is provided a kit for the identification of a patient's polymorphism pattern at; the rs2290573 polymorphism on the putative gene DKFZP434C131 in the 15q22.33 region, the CYP1A1 gene at the polymorphic site at position 6819 in sequence X02612; and the IL-1 beta gene, at the polymorphic site at position 1423 of sequence X04500, said kit comprising a means for determining a genetic polymorphism pattern at the above polymorphic sites. In a preferred embodiment, such kit may further comprise a DNA sample collecting means.

In a preferred embodiment the means for determining a genetic polymorphism pattern at the; rs2290573 polymorphism on the putative gene DKFZP434C131 in the 15q22.33 region, the CYP1A1gene at the polymorphic site at position 6819 in sequence X02612; and the IL-1 beta gene, at the polymorphic site at position 1423 of sequence X04500 comprise at least one genotyping oligonucleotide.

In particular, the means for determining a genetic polymorphism pattern at the polymorphic site of interest may comprise two genotyping oligonucleotides. Also, the means for determining a genetic polymorphism pattern at the polymorphic sites of interest may comprise at least one genotyping primer compositon comprising at least one genotyping oligonucleotide. In particular, the genotyping primer compositon may comprise at least two sets of allele specific primer pairs. Preferably, the two genotyping oligonucleotides are packaged in separate containers.

It is to be understood that the methods of the invention described herein generally may further comprise the use of a kit according to the invention. Generally, the methods of the invention may be performed ex-vivo, and such ex-vivo methods are specifically contemplated by the present invention. Also, where a method of the invention may include steps that may be practised on the human or animal body, methods that only comprise those steps which are not practised on the human or animal body are specifically contemplated by the present invention.

Effect(s) of the polymorphisms identified herein on expression of the rs2290573 polymorphism on the putative gene DKFZP434C131 in the 15q22.33 region, the CYP1A1 gene at the polymorphic site at position 6819 in sequence X02612; and the IL-1 beta gene, at the polymorphic site at position 1423 of sequence X04500 may be investigated by preparing recombinant cells and/or organisms, preferably recombinant animals, containing a polymorphic variant of the gene. As used herein, “expression” includes but is not limited to one or more of the following: transcription of the gene into precursor mRNA; splicing and other processing of the precursor mRNA to produce mature mRNA; mRNA stability; translation of the mature mRNA into expressed protein (including codon usage and tRNA availability); and glycosylation and/or other modifications of the translation product, if required for proper expression and function.

To prepare a recombinant cell of the invention, the desired isogene may be introduced into the cell in a vector such that the isogene remains extrachromosomal. In such a situation, the gene will be expressed by the cell from the extrachromosomal location. In a preferred embodiment, the isogene is introduced into a cell in such a way that it recombines with the endogenous gene present in the cell. Such recombination requires the occurrence of a double recombination event, thereby resulting in the desired gene polymorphism. Vectors for the introduction of genes both for recombination and for extrachromosomal maintenance are known in the art, and any suitable vector or vector construct may be used in the invention. Methods such as electroporation, particle bombardment, calcium phosphate co-precipitation and viral transduction for introducing DNA into cells are known in the art; therefore, the choice of method may lie with the competence and preference of the skilled practitioner.

Examples of cells into which the isogene may be introduced include, but are not limited to, continuous culture cells, such as COS, NIH/3T3, and primary or culture cells of the relevant tissue type, i.e., they express the isogene. Such recombinant cells can be used to compare the biological activities of the different protein variants.

Recombinant organisms, i.e., transgenic animals, expressing a variant gene are prepared using standard procedures known in the art. Preferably, a construct comprising the variant gene is introduced into a nonhuman animal or an ancestor of the animal at an embryonic stage, i.e., the one-cell stage, or generally not later than about the eight-cell stage. Transgenic animals carrying the constructs of the invention can be made by several methods known to those having skill in the art. One method involves transfecting into the embryo a retrovirus constructed to contain one or more insulator elements, a gene or genes of interest, and other components known to those skilled in the art to provide a complete shuttle vector harboring the insulated gene(s) as a transgene, see e.g., U.S. Pat. No. 5,610,053. Another method involves directly injecting a transgene into the embryo. A third method involves the use of embryonic stem cells.

Examples of animals, into which the isogenes may be introduced include, but are not limited to, mice, rats, other rodents, and nonhuman primates (see “The Introduction of Foreign Genes into Mice” and the cited references therein, In: Recombinant DNA, Eds. J. D. Watson, M. Gilman, J. Witkowski, and M. Zoller; W.H. Freeman and Company, New York, pages 254-272). Transgenic animals stably expressing a human isogene and producing human protein can be used as biological models for studying diseases related to abnormal expression and/or activity, and for screening and assaying various candidate drugs, compounds, and treatment regimens to reduce the symptoms or effects of these diseases.

TABLE 13
List of abbreviations Used in Gene Expression Section

| | |
|---|---|
| A | Absent |
| C | Cytarabine |
| AvDiff | Average Difference (overall intensity of probe set on |
| | Affymetrix array) |
| BKR | Best cytogenetic response, unconfirmed |
| CCyR | Complete cytogenetic response, confirmed |
| CHR | Complete hematological response |
| CML | Chronic myelogenous leukemia |
| CML-CP | Chronic myelogenous leukemia in chronic phase |
| EDTA | Ethylenediaminetetraacetic acid |
| GAPDH | Glyceraldehyde 3-phosphate dehydrogenase |
| IFN-α | Interferon-alpha |
| MCyR | Major cytogenetic response, confirmed (complete + |
| | partial) |
| MedDRA | Medical dictionary for regulatory activities |
| MKR | Major cytogenetic response, unconfirmed (complete + |
| | partial) |
| NCBI | National Center for Biotechnology Information |
| | (http://www.ncbi.nlm.nih.gov/) |
| NoCyR | Minimal or no response |
| OKR | Best cytogenetic response, confirmed |
| OMIM | Online Mendelian Inheritance in Man |
| P | Present |
| PD | Progressive disease |
| PG | Pharmacogenetics |
| Ph+ | Philadelphia chromosome positive |
| RT-PCR | Reverse transcription polymerase chain reaction |
| STI571 | Gleevec ™ /Glivec ®, Imatinib |
| TTP_C | Time-to-progression, censored (1 = no progression, |
| | 0 = progressed) |

Glossary and Definitions

The following definitions are provided to facilitate understanding of certain terms used frequently hereinbefore.

“Antibodies” as used herein includes polyclonal and monoclonal antibodies, chimeric, single chain and humanized antibodies, as well as Fab fragments, including the products of an Fab or other immunoglobulin expression library.

“Polynucleotide” generally refers to any polyribonucleotide (RNA) or polydeoxribonucleotide (DNA), which may be unmodified or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons.

“Polypeptide” refers to any polypeptide comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques that are well-known in the art. Such modifications are well-described in basic texts and in more detailed monographs, as well as in a voluminous research literature. Modifications may occur anywhere in a polypeptide, including the peptide backbone, the amino acid side-chains and the amino or carboxyl termini.

“Fragment” of a polypeptide sequence refers to a polypeptide sequence that is shorter than the reference sequence but that retains essentially the same biological function or activity as the reference polypeptide.

“Variant”refers to a polynucleotide or polypeptide that differs from a reference polynucleotide or polypeptide, but retains the essential properties thereof.

A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below.

A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gln-I Ser, Thr; Lys, Arg; and Phe and Tyr.

A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis.

Also included as variants are polypeptides having one or more post-translational modifications, for instance glycosylation, phosphorylation, methylation, ADP ribosylation and the like. Embodiments include methylation of the N-terminal amino acid, phosphorylations of serines and threonines and modification of C-terminal glycines.

“Allele” refers to one of two or more alternative forms of a gene occurring at a given locus in the genome.

“Polymorphism” refers to a variation in nucleotide sequence (and encoded polypeptide sequence, if relevant) at a given position in the genome within a population.

“Single Nucleotide Polymorphism” (SNP) refers to the occurrence of nucleotide variability at a single nucleotide position in the genome, within a population. An SNP may occur within a gene or within intergenic regions of the genome. SNPs can be assayed using Allele Specific Amplification (ASA). For the process at least 3 primers are required.

A common primer is used in reverse complement to the polymorphism being assayed. This common primer can be between 50 and 1500 bps from the polymorphic base. The other two (or more) primers are identical to each other except that the final 3′ base wobbles to match one of the two (or more) alleles that make up the polymorphism. Two (or more) PCR reactions are then conducted on sample DNA, each using the common primer and one of the Allele Specific Primers.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

“Homolog” is a generic term used in the art to indicate a polynucleotide or polypeptide sequence possessing a high degree of sequence relatedness to a reference sequence. Such relatedness may be quantified by determining the degree of identity and/or similarity between the two sequences as hereinbefore defined. Falling within this generic term are the terms “ortholog” and “paralog”. “Ortholog” refers to a polynucleotide or polypeptide that is the functional equivalent of the polynucleotide or polypeptide in another species. “Paralog” refers to a polynucleotide or polypeptide that within the same species which is functionally similar.

### References Cited

All publications and references, including but not limited to publications, patents, patent applications, GenBank accession, Unigene Cluster numbers and protein accession numbers, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. A method to predict which patients will respond to a tyrosine kinase inhibitor drug in Philadelphia chromosome positive leukemia patients comprising:
a) determining RNA expression levels in blood for a plurality of the 55 reporter genes shown in Tables 12A and 12B;
b) comparing patients gene expression profile to the mean complete cytogenetic response expression profiles shown in Tables 12A and 12B;
c) determining the Pearson correlation coefficient resulting from the comparison in (b);
d) determining that the patient will have complete cytogenetic response to the tyrosine kinase inhibitor if the correlation coefficient is equal to or greater than 0.57; and
e) determining that the patient will be a non-responder if the correlation coefficient is less than 0.57.

2. A method to predict which patients will respond to a tyrosine kinase inhibitor drug in Philadelphia chromosome positive leukemia patients comprising:
a) determining RNA expression levels in blood for a plurality of the 55 reporter genes shown in Tables 12A and 12B;
b) comparing patients gene expression profile to the mean complete cytogenetic response expression profiles shown in Tables 12A and 12B;
c) determining the Pearson correlation coefficient resulting from the comparison in (b);
d) determining that the patient will have complete cytogenetic response to the tyrosine kinase inhibitor if the correlation coefficient is equal to or greater than 0.54; and
e) determining that the patient will be a non-responder if the correlation coefficient is less than 0.54.

3. The method of claim 1 wherein the plurality of the 55 reporter genes comprises two or more of the 55 reporter genes shown in Tables 12A and 12B.

4. The method of claim 1 wherein the tyrosine kinase inhibitor is Imatinib mesylate (Imatinib or GLEEVEC® or GLIVEC® or STI571).

5. The method of claim 1 wherein only the 31 reporter genes in Table 12A are used.

6. A method for determining the responsiveness of an individual with Philadelphia chromosome positive leukemia to treatment with a tyrosine kinase inhibitor drug comprising:
a) determining for the two copies of the CSK gene, present in the individual, the identity of the nucleotide pair at the polymorphic site at position 36211 of sequence AC020705.4; and
b) assigning the individual to a good responder group if both pairs are AT, or if one pair is AT and one pair is GC, and to a low responder group if both pairs are GC.

7. A method for determining the responsiveness of an individual with Philadelphia chromosome positive leukemia to treatment with a tyrosine kinase inhibitor drug comprising:
a) determining for the two copies of the CYP1A1 gene, present in the individual, the identity of the nucleotide pair at the polymorphic site at position 6819 in sequence X02612; and
b) assigning the individual to a good responder group if both pairs are AT, and to a poor responder group if both pairs are GC, or if one is GC and one is AT.

8. A method for determining the responsiveness of an individual with Philadelphia chromosome positive leukemia to treatment with a tyrosine kinase inhibitor drug comprising:
a) determining for the two copies of the IL-1β gene, present in the individual, the identity of the nucleotide pair at position 1423 of sequence X04500; and
b) assigning the individual to a good responder group if both pairs are CG, and to a poor responder group if one pair is AT and one pair is CG or if both pairs are AT.

9. The method of claim 6 wherein the tyrosine kinase inhibitor is Imatinib mesylate (Imatinib or GLEEVEC® or GLIVEC® or STI571).

10. A method to determine the probability of a positive clinical response in a patient, with a tyrosine kinase inhibitor drug responsive disorder, to treatment with a tyrosine kinase inhibitor drug; comprising:
(a) obtaining a biological sample from the said patient,
(b) determining the levels of gene expression of two or more of the 55 reporter genes listed in Tables 12A and 12B in the sample from the patient, and
(c) comparing the levels of gene expression of the two or more genes determined in (b) to the levels of expression of the same genes as listed in Tables 12A and/or 12B and
(d) determining the degree of similarity between the levels of gene expression of the two or more genes determined in (c), and
(e) determining from the degree of similarity between the levels of gene expression of the two or more genes the probability that the patients will respond to a tyrosine kinase inhibitor drug.

11. The method of claim 10 wherein the tyrosine kinase inhibitor drug responsive disorder is Philadelphia chromosome positive leukemia.

12. The method of claim 11 wherein the tyrosine kinase inhibitor is Imatinib mesylate (Imatinib or GLEEVEC® or GLIVEC® or STI571).

13. The method of claim 10 wherein the biological sample is selected from the group consisting of; a tissue biopsy, blood, serum, plasma, lymph, ascitic fluid, cystic fluid, urine, sputum, stool, saliva, bronchial aspirate, CSF or hair.

14. A method according to claim 13, wherein the biological sample is a tissue biopsy cell sample or cells cultured therefrom.

15. A method according to claim 13, wherein the tissue biopsy is a biopsy of bone marrow or solid tissue.

16. A method according to claim 13 wherein the tissue biopsy comprises cells removed from a solid tumor.

17. A method according to claim 13 wherein the biological sample are blood cells.

18. A method according to claim 13 wherein said sample is a lysate of said cell sample.

19. The method of claim 10 wherein the level of gene expression is determined by measuring the level of transcription of the two or more genes in Tables 12A and/or 12B.

20. The method of claim 19 wherein the level of transcription is determined by measuring the level of mRNA of the two or more genes in Tables 12A and/or 12B.

21. The method of claim 19, wherein the level of transcription is determined by measuring the level of cDNA corresponding to the two or more genes in Tables 12A and/or 12B.

22. The method of claim 19 wherein the step of measuring further comprises amplifying the mRNA or cDNA.

23. The method of claim 19 wherein the level of transcription is determined by techniques selected from the group of Northern blot analysis, reverse transcriptase PCR, real-time PCR, RNAse protection, and microarray.

24. The method of claim 10 wherein the level of gene expression is determined for a plurality of the 55 reporter genes shown in Tables 12A and/or 12B.

25. The method of claim 24, wherein the plurality of the 55 reporter genes comprises the 31 genes shown in Table 12A.

26. The method of claim 24, wherein the plurality of the 55 reporter genes consists of the 31 genes shown in Table 12A.

27. The method of claim 10 wherein the degree of similarity in step (d) is determined by calculating a correlation coefficient whose value is a known function of the similarity of the values of gene expression.

28. The method of claim 27, wherein the correlation coefficient is the Pearson correlation coefficient.

29. The method of claim 28, wherein if the Pearson correlation coefficient between the Mean NoCyR values of the 31 reporter genes of Table 12A and the measured values of gene expression of the same genes from a patient with a tyrosine kinase inhibitor drug responsive disorder is greater than or equal to 0.54 the patient is classified as a non-responder to treatment with a tyrosine kinase inhibitor drug and if the Pearson correlation coefficient between the Mean NoCyR values of the 31 reporter genes of Table 12A and the measured values of gene expression of the same genes from a patient with a tyrosine kinase inhibitor drug responsive disorder is less than 0.54 the patient is classified as a responder to treatment with a tyrosine kinase inhibitor drug.

30. The method of claim 28, wherein if the Pearson correlation coefficient between the Mean NoCyR values of the 31 reporter genes of Table 12A and the measured values of gene expression of the same genes from a patient with a tyrosine kinase inhibitor drug responsive disorder is greater than or equal to 0.57 the patient is classified as a non-responder to treatment with a tyrosine kinase inhibitor drug and if the Pearson correlation coefficient between the Mean NoCyR values of the 31 reporter genes of Table 12A and the measured values of gene expression of the same genes from a patient with a tyrosine kinase inhibitor drug responsive disorder is less than 0.57 the patient is classified as a responder to treatment with a tyrosine kinase inhibitor drug.

31. The method of claim 10 wherein the method of determining the levels of gene expression of two or more of the 55 reporter genes listed in Tables 12A and/or 12B in the sample from the patient comprises determining presence and levels of expression of the polypeptides corresponding to the two or more of the 55 reporter genes listed in Tables 12A and/or 12B.

32. The method of claim 31, wherein the presence and the levels of expression of the polypeptides of the said genes are detected by using a reagent which specifically binds to said polypeptides

33. The method of claim 32, wherein the reagent is selected from the group consisting of an antibody, an antibody derivative, and an antibody fragment.

34. The method of claim 31 wherein the presence and the levels of expression of the polypeptides of the said genes are detected through Western blotting using a labeled probe specific for each polypeptide.

35. The method of claim 34, wherein the labeled probe is an antibody.

36. The method of claim 35, wherein the antibody is a monoclonal antibody.

37. A method for determining the responsiveness of a patient with a tyrosine kinase inhibitor drug responsive disorder, to treatment with a tyrosine kinase inhibitor drug comprising:
(a) determining for the two copies of the putative gene DKFZP434C131 in the 15q22.33 region, present in the said patient, the identity of the nucleotide pair at the polymorphic site referred to as the rs2290573 polymorphism; and
(b) assigning the individual to a good responder group if both pairs are AT, or if one pair is AT and one pair is GC, and to a low responder group if both pairs are GC.

38. A method for determining the responsiveness of a patient with a tyrosine kinase inhibitor drug responsive disorder, to treatment with a tyrosine kinase inhibitor drug comprising:
(a) determining for the two copies of the CYP1A1 gene, present in the said patient, the identity of the nucleotide pair at the polymorphic site at position 6819 in sequence X02612; and
(b) assigning the individual to a good responder group if both pairs are AT, and to a poor responder group if both pairs are GC, or if one is GC and one is AT.

39. A method for determining the responsiveness of a patient with a tyrosine kinase inhibitor drug responsive disorder, to treatment with a tyrosine kinase inhibitor drug comprising:
(a) determining for the two copies of the IL-1 beta gene, present in the patient, the identity of the nucleotide pair at position 1423 of sequence X04500; and
(b) assigning the individual to a good responder group if both pairs are CG, and to a poor responder group if one pair is AT and one pair is CG or if both pairs are AT.

40. The methods of claim 37 wherein the tyrosine kinase inhibitor drug responsive disorder is Philadelphia chromosome positive leukemia.

41. The methods of claim 37 wherein the tyrosine kinase inhibitor is Imatinib mesylate (Imatinib or GLEEVEC® or GLIVEC® or STI571).

42. A method according to claim 10 wherein said method is performed ex-vivo.

43. A kit for determining the responsiveness to treatment with a tyrosine kinase inhibitor drug of a patient with a tyrosine kinase inhibitor drug responsive disorder, comprising:
a means for detecting the polypeptides corresponding to the two or more of the 55 reporter genes listed in Tables 12A and/or 3B.

44. A kit according to claim 43, wherein the means for detecting the polypeptides comprise antibodies, antibody derivatives, or antibody fragments.

45. A kit according to claim 43 wherein the polypeptides are detected through Western blotting utilizing a labeled antibody.

46. A kit according to claim 43 further comprising means for obtaining a biological sample of the patient.

47. A kit according to claim 43 further comprising a container suitable for containing the means for detecting the polypeptides and the biological sample of the patient.

48. A kit according to claim 43 further comprising instructions for use and interpretation of the kit results.

49. A kit for use in determining treatment strategy for a patient with a tyrosine kinase inhibitor drug responsive disorder, comprising:
(a) a means for detecting the polypeptides corresponding to the two or more of the 55 reporter genes listed in Tables 12A and/or 12B;
(b) a container suitable for containing the said means and the biological sample of the patient comprising the polypeptides wherein the means can form complexes with the polypeptides;
(c) a means to detect the complexes of (b); and optionally
(d) instructions for use and interpretation of the kit results.

50. A kit for determining the responsiveness to treatment with a tyrosine kinase inhibitor drug, of a patient with a tyrosine kinase inhibitor drug responsive disorder; comprising:
a means for measuring the level of transcription of the two or more genes listed in Tables 12A and/or 12B.

51. A kit according to claim 50, wherein the means for measuring the level of transcription comprise oligonucleotides or polynucleotides able to bind to the transcription products of said genes.

52. A kit according to claim 51, wherein the oligonucleotides or polynucleotides are able to bind mRNA or cDNA corresponding to said genes.

53. A kit according to claim 50 wherein the level of transcription is determined by techniques selected from the group of Northern blot analysis, reverse transcriptase PCR, real-time PCR, RNAse protection, and microarray.

54. A kit according to claim 50 further comprising means for obtaining a biological sample of the patient.

55. A kit according to claim 50 further comprising a container suitable for containing the means for measuring the level of transcription and the biological sample of the patient.

56. A kit according to claim 50, further comprising instructions for use and interpretation of the kit results.

57. A kit for determining the responsiveness to treatment with a tyrosine kinase inhibitor drug, of a patient with a tyrosine kinase inhibitor drug responsive disorder; comprising:
(a) a number of oligonucleotides or polynucleotides able to bind to the transcription products of the two or more genes listed in Tables 12A and/or 12B;
(b) a container suitable for containing the oligonucleotides or polynucleotides and the biological sample of the patient comprising the transcription products wherein the oligonucleotides or polynucleotide can bind to the transcription products;
(c) means to detect the binding of (b); and optionally
(d) instructions for use and interpretation of the kit results.

58. A method according to claim 10 wherein the determination step (b) further comprises the use a kit according to any of claims 43 to 57.

59. A kit for the identification of a polymorphic site of the putative gene DKFZP434C131 in the 15q22.33 region of a patient with a tyrosine kinase inhibitor drug responsive disorder, said kit comprising a means for determining the genetic polymorphism pattern at the rs2290573CSK polymorphic site of the putative gene DKFZP434C131 in the 15q22.33 region.

60. A kit for the identification a polymorphism pattern at the CYP1A1 gene of a patient with a tyrosine kinase inhibitor drug responsive disorder, said kit comprising a means for determining the genetic polymorphism pattern at the CYP1A1 gene polymorphic site at position 6819 in sequence X02612.

61. A kit for the identification of a polymorphism pattern at the IL-1 beta gene of a patient with a tyrosine kinase inhibitor drug responsive disorder, said kit comprising a means for determining the genetic polymorphism pattern at the IL-1 beta gene at position 1423 in sequence X04500.

62. A kit according to claim 59 further comprising a means for obtaining a biological sample of the patient.

63. A kit according to claim 62, wherein the means comprises a DNA sample collecting means.

64. A kit according to claim 59 wherein the means for determining a genetic polymorphism pattern at the specific polymorphic site comprise at least one gene specific genotyping oligonucleotide.

65. A kit according to claim 59 wherein the means for determining a genetic polymorphism pattern at the specific polymorphic site comprise two gene specific genotyping oligonucleotides.

66. A kit according to claim 59 wherein the means for determining a genetic polymorphism pattern at the polymorphic site comprise at least one gene specific genotyping primer composition comprising at least one gene specific genotyping oligonucleotide.

67. A kit according to claim 66, wherein the gene specific genotyping primer composition comprises at least two sets of allele specific primer pairs.

68. A kit according to claim 67, wherein the two allele specific genotyping oligonucleotides are packaged in separate containers.

69. A method according to claim 37 wherein the determination step (a) further comprises the use a kit according to claim 50.
